(19) United States
(12) Patent Application Publication
(10) Pub. No.: US 2003/0017457 A1
(43) Pub. Date: Jan. 23, 2003

(54) Novel polynucleotides and polypeptides encoded thereby

(76) Inventors: Elma R. Fernandes,, Branford, CT (US); Corine A.M. Vernet, North Branford, CT (US); Vishnu S. Mishnu, Gainesville, FL (US); Martin D. Leach, Madison, CT (US); Richard A. Shimkets, West Haven, CT (US); Bryan D. Zerhusen, Branford, CT (US); Ramesha Kekuda, Branford, CT (US)

Correspondence Address: Ivor R. Elrifi, MINTZ, LEVIN, COHN, FERRIS,, GLOVSKY AND POPEO, P.C., One Financial Center, Boston, MA, 02111, US

(21) Appl. No.: 09/826,734

(22) Filed: Apr. 5, 2001

Related U.S. Application Data
(60) Provisional application No. 60/195,576, filed on Apr. 6, 2000.

(51) Int. Cl. C12Q 1/68; C07H 21/04; C12N 9/00; C12P 21/02; C12N 5/06

(52) U.S. Cl. 435/6; 435/183; 435/320.1; 435/325; 435/69.1; 536/23.2

(57) ABSTRACT

The present invention provides ORFX, a novel isolated polypeptide, as well as a polynucleotide encoding ORFX and antibodies that immunospecifically bind to ORFX or any derivative, variant, mutant, or fragment of the ORFX polypeptide, polynucleotide or antibody. The invention additionally provides methods in which the ORFX polypeptide, polynucleotide and antibody are used in detection and treatment of a broad range of pathological states, as well as to others uses.

### RELATED APPLICATIONS

This application claims priority from U.S. Ser. No. 60/195,576, filed Apr. 6, 2000 and incorporates the reference herein in its entirety.

### BACKGROUND OF THE INVENTION

The invention relates generally to nucleic acids and polypeptides encoded thereby, and methods of using these nucleic acids and polypeptides. The contents of this application are incorporated herein by reference in their entirety.

### SUMMARY OF THE INVENTION

The invention is based in part on the discovery of nucleic acids that include open reading frames encoding novel polypeptides, and on the polypeptides encoded thereby. The nucleic acids and polypeptides are collectively referred to herein as “ORFX”.

Accordingly, in one aspect, the invention provides an isolated nucleic acid molecule that includes the sequence of any of SEQ ID NO: 2n-1, wherein n is an integer between 1-132, that encodes a novel polypeptide, or a fragment, homolog, analog or derivative thereof. The nucleic acid can include, e.g., a nucleic acid sequence encoding a polypeptide at least 85% identical to a polypeptide comprising the amino acid sequences of SEQ ID NO: 2n, wherein n is an integer between 1-132. The nucleic acid can be, e.g., a genomic DNA fragment, or a cDNA molecule.

Also included in the invention is a vector containing one or more of the nucleic acids described herein, and a cell containing the vectors or nucleic acids described herein.

The invention is also directed to host cells transformed with a vector comprising any of the nucleic acid molecules described above.

In another aspect, the invention includes a pharmaceutical composition that includes an ORFX nucleic acid and a pharmaceutically acceptable carrier or diluent.

In a further aspect, the invention includes a substantially purified ORF polypeptide, e.g., any of the ORFX polypeptides encoded by an ORFX nucleic acid, and fragments, homologs, analogs, and derivatives thereof. The invention also includes a pharmaceutical composition that includes a ORFX polypeptide and a pharmaceutically acceptable carrier or diluent.

In a still a further aspect, the invention provides an antibody that binds specifically to an ORFX polypeptide. The antibody can be, e.g., a monoclonal or polyclonal antibody, and fragments, homologs, analogs, and derivatives thereof. The invention also includes a pharmaceutical composition including ORFX antibody and a pharmaceutically acceptable carrier or diluent. The invention is also directed to isolated antibodies that bind to an epitope on a polypeptide encoded by any of the nucleic acid molecules described above.

The invention also includes kits comprising any of the pharmaceutical compositions described above.

The invention further provides a method for producing an ORFX polypeptide by providing a cell containing a ORFX nucleic acid, e.g., a vector that includes a ORFX nucleic acid, and culturing the cell under conditions sufficient to express the ORFX polypeptide encoded by the nucleic acid. The expressed ORFX polypeptide is then recovered from the cell. Preferably, the cell produces little or no endogenous ORFX polypeptide. The cell can be, e.g., a prokaryotic cell or eukaryotic cell.

The invention is also directed to methods of identifying an ORFX polypeptide or nucleic acids in a sample by contacting the sample with a compound that specifically binds to the polypeptide or nucleic acid, and detecting complex formation, if present.

The invention further provides methods of identifying a compound that modulates the activity of a ORFX polypeptide by contacting ORFX polypeptide with a compound and determining whether the ORFX polypeptide activity is modified.

The invention is also directed to compounds that modulate ORFX polypeptide activity identified by contacting a ORFX polypeptide with the compound and determining whether the compound modifies activity of the ORFX polypeptide, binds to the ORFX polypeptide, or binds to a nucleic acid molecule encoding a ORFX polypeptide.

In a another aspect, the invention provides a method of determining the presence of or predisposition of an ORFX-associated disorder in a subject. The method includes providing a sample from the subject and measuring the amount of ORFX polypeptide in the subject sample. The amount of ORFX polypeptide in the subject sample is then compared to the amount of ORFX polypeptide in a control sample. An alteration in the amount of ORFX polypeptide in the subject protein sample relative to the amount of ORFX polypeptide in the control protein sample indicates the subject has a tissue proliferation-associated condition. A control sample is preferably taken from a matched individual, i.e., an individual of similar age, sex, or other general condition but who is not suspected of having a tissue proliferation-associated condition. Alternatively, the control sample may be taken from the subject at a time when the subject is not suspected of having a tissue proliferation-associated disorder. In some embodiments, the ORFX is detected using a ORFX antibody.

In a further aspect, the invention provides a method of determining the presence of or predisposition of an ORFX-associated disorder in a subject. The method includes providing a nucleic acid sample, e.g., RNA or DNA, or both, from the subject and measuring the amount of the ORFX nucleic acid in the subject nucleic acid sample. The amount of ORFX nucleic acid sample in the subject nucleic acid is then compared to the amount of an ORFX nucleic acid in a control sample. An alteration in the amount of ORFX nucleic acid in the sample relative to the amount of ORFX in the control sample indicates the subject has a tissue proliferation-associated disorder.

In a still further aspect, the invention provides method of treating or preventing or delaying a ORFX-associated disorder. The method includes administering to a subject in which such treatment or prevention or delay is desired a ORFX nucleic acid, a ORFX polypeptide, or an ORFX antibody in an amount sufficient to treat, prevent, or delay a tissue proliferation-associated disorder in the subject.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention provides novel polypeptides and nucleotides encoded thereby. The polynucleotides and their encoded polypeptides can be grouped according to the functions played by their gene products. Such functions include, e.g. structural proteins, proteins associated with metabolic pathways such as fatty acid metabolism, glycolysis, intermediary metabolism, calcium metabolism, proteases, and amino acid metabolism.

Included in the invention are 132 novel nucleic acid sequences and their encoded polypeptides. The sequences are collectively referred to as “ORFX nucleic acids” or ORFX polynucleotides” and the corresponding encoded polypeptide is referred to as a “ORFX polypeptide” or “ORFX protein”. For example, an ORFX nucleic acid according to the invention is a nucleic acid including an ORF1 nucleic acid, and an ORF polypeptide according to the invention is a polypeptide that includes the amino acid sequence of an ORF1 polypeptide. Unless indicated otherwise, “ORFX” is meant to refer to any of the ORF1-132 sequences disclosed herein.

In general, for an ORFn according to the invention (wherein n is any integer from 1 to 132), a nucleic acid corresponding to the ORF is SEQ ID NO: 2n-1, and an amino acid sequence encoded by the ORF is SEQ ID NO: 2n. For example, a nucleic acid sequence corresponding to an ORF1 nucleic acid is SEQ ID NO: 1, and a polypeptide sequence corresponding to an ORF1 polypeptide is SEQ ID NO: 2. Similarly, a nucleic acid sequence corresponding to an ORF4 nucleic acid is SEQ ID NO: 7, and a polypeptide sequence corresponding to an ORF4 polypeptide is SEQ ID NO: 8. Nucleic acid sequences and polypeptide sequences for ORFX nucleic acids according to the invention are provided in Table 1. Also shown in Table 1 are polypeptides related to the indicated ORFX sequence.

To determine similarity to previously described proteins, polypeptides encoded by ORFX DNA sequences were tested using the Framesearch Algorithm against a nonredundant version of the GenPept Database from NCBI/Genbank. DNA sequences that had a score of ‘90’ or above (Framesearch algorithm score, Edelman et. al. GCG Genetics) to a known protein were selected. Percent sequence homology of ORFX polypeptides to known protein is provided in field 223 in section of the specification entitled “Disclosed Sequences of ORFX Nucleic Acid and Polypeptide Sequences.” Open reading frames were extended beyond the region of the protein matched using standard DNA translation and codon tables. Novel proteins that lacked a protein match were translated against the standard genetic codons and proteins with an ORF at least 80 amino acids.

ORFX nucleic acids, and their encoded polypeptides, according to the invention are useful in a variety of applications and contexts. For example, various ORFX nucleic acids and polypeptides according to the invention are useful, inter alia, as novel members of the protein families according to the presence of domains and sequence relatedness to previously described proteins

ORFX nucleic acids and polypeptides according to the invention can also be used to identify cell types for an indicated ORFX according to the invention. Additional utilities for ORFX nucleic acids and polypeptides according to the invention are disclosed herein.

ORFX Nucleic Acids

The novel nucleic acids of the invention include those that encode an ORFX or ORFX-like protein, or biologically active portions thereof. The nucleic acids include nucleic acids encoding polypeptides that include the amino acid sequence of one or more of SEQ ID NO: 2n, wherein n=1 to 132. The encoded polypeptides can thus include, e.g., the amino acid sequences of SEQ ID NO: 2, 4, 6, 8, 10, . . . 258, 260, 262, and/or 264.

In some embodiments, a nucleic acid encoding a polypeptide having the amino acid sequence of one or more of SEQ ID NO: 2n (wherein n=1 to 132) includes the nucleic acid sequence of any of SEQ ID NO: 2n-1 (wherein n=1 to 132), or a fragment thereof. Additionally, the invention includes mutant or variant nucleic acids of any of SEQ ID NO: 2n-1 (wherein n=1 to 132), or a fragment thereof, any of whose bases may be changed from the disclosed sequence while still encoding a protein that maintains its ORFX-like activities and physiological functions. The invention further includes the complement of the nucleic acid sequence of any of SEQ ID NO: 2n-1 (wherein n=1 to 132), including fragments, derivatives, analogs and homolog thereof. The invention additionally includes nucleic acids or nucleic acid fragments, or complements thereto, whose structures include chemical modifications.

Also included are nucleic acid fragments sufficient for use as hybridization probes to identify ORFX-encoding nucleic acids (e.g., ORFX mRNA) and fragments for use as polymerase chain reaction (PCR) primers for the amplification or mutation of ORFX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

“Probes” refer to nucleic acid sequences of variable length, preferably between at least about 10 nucleotides (nt), 100 nt, or as many as about, e.g., 6,000 nt, depending on use. Probes are used in the detection of identical, similar, or complementary nucleic acid sequences. Longer length probes are usually obtained from a natural or recombinant source, are highly specific and much slower to hybridize than oligomers. Probes may be single- or double-stranded and designed to have specificity in PCR, membrane-based hybridization technologies, or ELISA-like technologies.

An “isolated” nucleic acid molecule is one that is separated from other nucleic acid molecules that are present in the natural source of the nucleic acid. Examples of isolated nucleic acid molecules include, but are not limited to, recombinant DNA molecules contained in a vector, recombinant DNA molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated ORFX nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having the nucleotide sequence of SEQ ID NO: 2n-1 (wherein n=1 to 132), or a complement of any of this nucleotide sequence, can be isolated using standard molecular biology techniques and the sequence information provided herein. Using all or a portion of the nucleic acid sequence of any of SEQ ID NO: 2n-1 (wherein n=1 to 132) as a hybridization probe, ORFX nucleic acid sequences can be isolated using standard hybridization and cloning techniques (e.g., as described in Sambrook et al., eds., MOLECULAR CLONING: A LABORATORY MANUAL 2^{nd} Ed., Cold Spring Harbor Laboratory Press, Cold Spring Harbor, N.Y., 1989; and Ausubel, et al., eds., CURRENT PROTOCOLS IN MOLECULAR BIOLOGY, John Wiley & Sons, New York, NY, 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to ORFX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at lease 6 contiguous nucleotides of any of SEQ ID NO: 2n-1 (wherein n=1 to 132), or a complement thereof. Oligonucleotides may be chemically synthesized and may be used as probes.

In another embodiment, an isolated nucleic acid molecule of the invention comprises a nucleic acid molecule that is a complement of the nucleotide sequence shown in any of SEQ ID NO: 2n-1 (wherein n=1 to 132). In another embodiment, an isolated nucleic acid molecule of the invention comprises a nucleic acid molecule that is a complement of the nucleotide sequence shown in any of SEQ ID NO: 2n-1 (wherein n=1 to 132), or a portion of this nucleotide sequence. A nucleic acid molecule that is complementary to the nucleotide sequence shown in is one that is sufficiently complementary to the nucleotide sequence shown in of any of SEQ ID NO: 2n-1 (wherein n=1 to 132) that it can hydrogen bond with little or no mismatches to the nucleotide sequence shown in of any of SEQ ID NO: 2n-1 (wherein n=1 to 132), thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Von der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule of the invention can comprise only a portion of the nucleic acid sequence of any of SEQ ID NO: 2n-1 (wherein n=1 to 132), e.g., a fragment that can be used as a probe or primer, or a fragment encoding a biologically active portion of ORFX. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, 85%, 90%, 95%, 98%, or even 99% identity (with a preferred identity of 80-99%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below. An exemplary program is the Gap program (Wisconsin Sequence Analysis Package, Version 8 for UNIX, Genetics Computer Group, University Research Park, Madison, Wis.) using the default settings, which uses the algorithm of Smith and Waterman (Adv. Appl. Math., 1981, 2: 482-489, which is incorporated herein by reference in its entirety).

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of ORFX polypeptide. Isoforms can be expressed in different tissues of the same organism as a result of, for example, alternative splicing of RNA. Alternatively, isoforms can be encoded by different genes. In the present invention, homologous nucleotide sequences include nucleotide sequences encoding for a ORFX polypeptide of species other than humans, including, but not limited to, mammals, and thus can include, e.g., mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the nucleotide sequence encoding human ORFX protein. Homologous nucleic acid sequences include those nucleic acid sequences that encode conservative amino acid substitutions (see below) in any of SEQ ID NO: 2n (wherein n=1 to 132) as well as a polypeptide having ORFX activity. Biological activities of the ORFX proteins are described below. A homologous amino acid sequence does not encode the amino acid sequence of a human ORFX polypeptide.

The nucleotide sequence determined from the cloning of the human ORFX gene allows for the generation of probes and primers designed for use in identifying the cell types disclosed and/or cloning ORFX homologues in other cell types, e.g., from other tissues, as well as ORFX homologues from other mammals. The probe/primer typically comprises a substantially purified oligonucleotide. The oligonucleotide typically comprises a region of nucleotide sequence that hybridizes under stringent conditions to at least about 12, 25, 50, 100, 150, 200, 250, 300, 350 or 400 or more consecutive sense strand nucleotide sequence of SEQ ID NO: 2n-1 (wherein n=1 to 132); or an anti-sense strand nucleotide sequence of SEQ ID NO: 2n-1 (wherein n=1 to 132); or of a naturally occurring mutant of SEQ ID NO: 2n-1 (wherein n=1 to 132).

Probes based on the human ORFX nucleotide sequence can be used to detect transcripts or genomic sequences encoding the same or homologous proteins. In various embodiments, the probe further comprises a label group attached thereto, e.g., the label group can be a radioisotope, a fluorescent compound, an enzyme, or an enzyme co-factor. Such probes can be used as a part of a diagnostic test kit for identifying cells or tissue which misexpress a ORFX protein, such as by measuring a level of a ORFX-encoding nucleic acid in a sample of cells from a subject e.g., detecting ORFX mRNA levels or determining whether a genomic ORFX gene has been mutated or deleted.

“A polypeptide having a biologically active portion of ORFX” refers to polypeptides exhibiting activity similar, but not necessarily identical to, an activity of a polypeptide of the present invention, including mature forms, as measured in a particular biological assay, with or without dose dependency. A nucleic acid fragment encoding a “biologically active portion of ORFX” can be prepared by isolating a portion of SEQ ID NO: 2n-1 (wherein n=1 to 132), that encodes a polypeptide having a ORFX biological activity (biological activities of the ORFX proteins are summarized in Table 1), expressing the encoded portion of ORFX protein (e.g., by recombinant expression in vitro) and assessing the activity of the encoded portion of ORFX. For example, a nucleic acid fragment encoding a biologically active portion of ORFX can optionally include a domain as shown in Table 1, column 4.

ORFX Variants

The invention further encompasses nucleic acid molecules that differ from the disclosed ORFX nucleotide sequences due to degeneracy of the genetic code. These nucleic acids thus encode the same ORFX protein as that encoded by the nucleotide sequence shown in SEQ ID NO: 2n-1 (wherein n=1 to 132). In another embodiment, an isolated nucleic acid molecule of the invention has a nucleotide sequence encoding a protein having an amino acid sequence shown in any of SEQ ID NO: 2n (wherein n=1 to 132).

In addition to the human ORFX nucleotide sequence shown in any of SEQ ID NO: 2n-1 (wherein n=1 to 132), it will be appreciated by those skilled in the art that DNA sequence polymorphisms that lead to changes in the amino acid sequences of ORFX may exist within a population (e.g., the human population). Such genetic polymorphism in the ORFX gene may exist among individuals within a population due to natural allelic variation. As used herein, the terms “gene” and “recombinant gene” refer to nucleic acid molecules comprising an open reading frame encoding a ORFX protein, preferably a mammalian ORFX protein. Such natural allelic variations can typically result in 1-5% variance in the nucleotide sequence of the ORFX gene. Any and all such nucleotide variations and resulting amino acid polymorphisms in ORFX that are the result of natural allelic variation and that do not alter the functional activity of ORFX are intended to be within the scope of the invention.

Moreover, nucleic acid molecules encoding ORFX proteins from other species, and thus that have a nucleotide sequence that differs from the human sequence of any of SEQ ID NO: 2n-1 (wherein n=1 to 132), are intended to be within the scope of the invention. Nucleic acid molecules corresponding to natural allelic variants and homologues of the ORFX cDNAs of the invention can be isolated based on their homology to the human ORFX nucleic acids disclosed herein using the human cDNAs, or a portion thereof, as a hybridization probe according to standard hybridization techniques under stringent hybridization conditions.

In another embodiment, an isolated nucleic acid molecule of the invention is at least 6 nucleotides in length and hybridizes under stringent conditions to the nucleic acid molecule comprising the nucleotide sequence of any of SEQ ID NO: 2n-1 (wherein n 1 to 132). In another embodiment, the nucleic acid is at least 10, 25, 50, 100, 250, 500 or 750 nucleotides in length. In another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding ORFX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× SSC, 50 mM Tris-HCl (pH 7.5), 1 mM EDTA, 0.02% PVP, 0.02% Ficoll, 0.02% BSA, and 500 mg/ml denatured salmon sperm DNA at 65° C. This hybridization is followed by one or more washes in 0.2× SSC, 0.01% BSA at 50° C. An isolated nucleic acid molecule of the invention that hybridizes under stringent conditions to the sequence of any of SEQ ID NO: 2n-1 (wherein n=1 to 132) corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of any of SEQ ID NO: 2n-1 (wherein n=1 to 132), or fragments, analogs or derivatives thereof, under conditions of moderate stringency is provided. A non-limiting example of moderate stringency hybridization conditions are hybridization in 6× SSC, 5× Denhardt's solution, 0.5% SDS and 100 mg/ml denatured salmon sperm DNA at 55° C., followed by one or more washes in 1× SSC, 0.1% SDS at 37° C. Other conditions of moderate stringency that may be used are well known in the art. See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY.

In a third embodiment, a nucleic acid that is hybridizable to the nucleic acid molecule comprising the nucleotide sequence of any of SEQ ID NO: 2n-1 (wherein n=1 to 132), or fragments, analogs or derivatives thereof, under conditions of low stringency, is provided. A non-limiting example of low stringency hybridization conditions are hybridization in 35% formamide, 5× SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2× SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981, Proc Natl Acad Sci USA 78: 6789-6792.

Conservative Mutations

In addition to naturally-occurring allelic variants of the ORFX sequence that may exist in the population, the skilled artisan will further appreciate that changes can be introduced by mutation into the nucleotide sequence of any of SEQ ID NO: 2n-1 (wherein n=1 to 132), thereby leading to changes in the amino acid sequence of the encoded ORFX protein, without altering the functional ability of the ORFX protein. For example, nucleotide substitutions leading to amino acid substitutions at “non-essential” amino acid residues can be made in the sequence of any of SEQ ID NO: 2n-1 (wherein n=1 to 132). A “non-essential” amino acid residue is a residue that can be altered from the wild-type sequence of ORFX without altering the biological activity, whereas an “essential” amino acid residue is required for biological activity. For example, amino acid residues that are conserved among the ORFX proteins of the present invention, are predicted to be particularly unamenable to alteration.

Amino acid residues that are conserved among members of an ORFX family members are predicted to be less amenable to alteration. For example, an ORFX protein according to the present invention can contain at least one domain (e.g., as shown in Table 1) that is a typically conserved region in an ORFX family member. As such, these conserved domains are not likely to be amenable to mutation. Other amino acid residues, however, (e.g., those that are not conserved or only semi-conserved among members of the ORFX family) may not be as essential for activity and thus are more likely to be amenable to alteration.

Another aspect of the invention pertains to nucleic acid molecules encoding ORFX proteins that contain changes in amino acid residues that are not essential for activity. Such ORFX proteins differ in amino acid sequence from any of any of SEQ ID NO: 2n (wherein n=1 to 132), yet retain biological activity. In one embodiment, the isolated nucleic acid molecule comprises a nucleotide sequence encoding a protein, wherein the protein comprises an amino acid sequence at least about 75% homologous to the amino acid sequence of any of SEQ ID NO: 2n (wherein n=1 to 132). Preferably, the protein encoded by the nucleic acid is at least about 80% homologous to any of SEQ ID NO: 2n (wherein n=1 to 132), more preferably at least about 90%, 95%, 98%, and most preferably at least about 99% homologous to SEQ ID NO: 2.

An isolated nucleic acid molecule encoding a ORFX protein homologous to the protein of any of SEQ ID NO: 2n (wherein n=1 to 132) can be created by introducing one or more nucleotide substitutions, additions or deletions into the corresponding nucleotide sequence, i.e. SEQ ID NO: 2n-1 for the corresponding n, such that one or more amino acid substitutions, additions or deletions are introduced into the encoded protein.

Mutations can be introduced into SEQ ID NO: 2n-1 (wherein n=1 to 132) by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in ORFX is replaced with another amino acid residue from the same side chain family. Alternatively, in another embodiment, mutations can be introduced randomly along all or part of a ORFX coding sequence, such as by saturation mutagenesis, and the resultant mutants can be screened for ORFX biological activity to identify mutants that retain activity. Following mutagenesis of SEQ ID NO: 2n-1 (wherein n=1 to 132) the encoded protein can be expressed by any recombinant technology known in the art and the activity of the protein can be determined.

In one embodiment, a mutant ORFX protein can be assayed for (1) the ability to form protein:protein interactions with other ORFX proteins, other cell-surface proteins, or biologically active portions thereof, (2) complex formation between a mutant ORFX protein and a ORFX receptor; (3) the ability of a mutant ORFX protein to bind to an intracellular target protein or biologically active portion thereof; (e.g., avidin proteins); (4) the ability to bind BRA protein; or (5) the ability to specifically bind an anti-ORFX protein antibody.

Antisense

Another aspect of the invention pertains to isolated antisense nucleic acid molecules that are hybridizable to or complementary to the nucleic acid molecule comprising the nucleotide sequence of SEQ ID NO: 2n-1 (wherein n=1 to 132), or fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to at least about 10, 25, 50, 100, 250 or 500 nucleotides or an entire ORFX coding strand, or to only a portion thereof. Nucleic acid molecules encoding fragments, homologs, derivatives and analogs of a ORFX protein of any of SEQ ID NO: 2n (wherein n=1 to 132) or antisense nucleic acids complementary to a ORFX nucleic acid sequence of SEQ ID NO: 2n-1 (wherein n=1 to 132) are additionally provided.

In one embodiment, an antisense nucleic acid molecule is antisense to a “coding region” of the coding strand of a nucleotide sequence encoding ORFX. The term “coding region” refers to the region of the nucleotide sequence comprising codons which are translated into amino acid residues (e.g., the protein coding region of a human ORFX that corresponds to any of SEQ ID NO: 2n (wherein n=1 to 132)). In another embodiment, the antisense nucleic acid molecule is antisense to a “noncoding region” of the coding strand of a nucleotide sequence encoding ORFX. The term “noncoding region” refers to 5′ and 3′ sequences which flank the coding region that are not translated into amino acids (i.e., also referred to as 5′ and 3′ untranslated regions).

Given the coding strand sequences encoding ORFX disclosed herein (e.g., SEQ ID NO: 2n-1 (wherein n=1 to 132) ), antisense nucleic acids of the invention can be designed according to the rules of Watson and Crick or Hoogsteen base pairing. The antisense nucleic acid molecule can be complementary to the entire coding region of ORFX mRNA, but more preferably is an oligonucleotide that is antisense to only a portion of the coding or noncoding region of ORFX mRNA. For example, the antisense oligonucleotide can be complementary to the region surrounding the translation start site of ORFX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcyto sine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a ORFX protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res 15: 6625-6641). The antisense nucleic acid molecule can also comprise a 2′-o-methylribonucleotide (Inoue et al. (1987) Nucleic Acids Res 15: 6131-6148) or a chimeric RNA -DNA analogue (Inoue et al. (1987) FEBS Lett 215: 327-330).

Ribozymes and PNA Moieties

Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In still another embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave ORFX mRNA transcripts to thereby inhibit translation of ORFX mRNA. A ribozyme having specificity for a ORFX-encoding nucleic acid can be designed based upon the nucleotide sequence of a ORFX DNA disclosed herein (i e., SEQ ID NO: 2n-1 (wherein n=1 to 132)). For example, a derivative of a Tetrahymena L-19 IVS RNA can be constructed in which the nucleotide sequence of the active site is complementary to the nucleotide sequence to be cleaved in a ORFX-encoding mRNA. See, e.g., Cech et al. U.S. Pat. No. 4,987,071; and Cech et al. U.S. Pat. No. 5,116,742. Alternatively, ORFX mRNA can be used to select a catalytic RNA having a specific ribonuclease activity from a pool of RNA molecules. See, e.g., Bartel et al., (1993) Science 261:1411-1418.

Alternatively, ORFX gene expression can be inhibited by targeting nucleotide sequences complementary to the regulatory region of the ORFX (e.g., the ORFX promoter and/or enhancers) to form triple helical structures that prevent transcription of the ORFX gene in target cells. See generally, Helene. (1991) Anticancer Drug Des. 6: 569-84; Helene. etal. (1992) Ann. N.Y. Acad. Sci. 660:27-36; and Maher (1992) Bioassays 14: 807-15.

In various embodiments, the nucleic acids of ORFX can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorg Med Chem 4: 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) above; Perry-O'Keefe et al. (1996) PNAS 93: 14670-675.

PNAs of ORFX can be used in therapeutic and diagnostic applications. For example, PNAs can be used as antisense or antigene agents for sequence-specific modulation of gene expression by, e.g., inducing transcription or translation arrest or inhibiting replication. PNAs of ORFX can also be used, e.g., in the analysis of single base pair mutations in a gene by, e.g., PNA directed PCR clamping; as artificial restriction enzymes when used in combination with other enzymes, e.g., S1 nucleases (Hyrup B. (1996) above); or as probes or primers for DNA sequence and hybridization (Hyrup et al. (1996), above; Perry-O'Keefe (1996), above).

In another embodiment, PNAs of ORFX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of ORFX can be generated that may combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996) above). The synthesis of PNA-DNA chimeras can be performed as described in Hyrup (1996) above and Finn et al. (1996) Nucl Acids Res 24: 3357-63. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA (Mag et al. (1989) Nucl Acid Res 17: 5973-88). PNA monomers are then coupled in a stepwise manner to produce a chimeric molecule with a 5′ PNA segment and a 3′ DNA segment (Finn et al. (1996) above). Alternatively, chimeric molecules can be synthesized with a 5′ DNA segment and a 3′ PNA segment. See, Petersen et al. (1975) Bioorg Med Chem Lett 5: 1119-11124.

In other embodiments, the oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810) or the blood-brain barrier (see, e.g., PCT Publication No. WO89/10134). In addition, oligonucleotides can be modified with hybridization triggered cleavage agents (See, e.g., Krol et al., 1988, BioTechniques 6:958-976) or intercalating agents. (See, e.g., Zon, 1988, Pharm. Res. 5: 539-549). To this end, the oligonucleotide may be conjugated to another molecule, e.g., a peptide, a hybridization triggered cross-linking agent, a transport agent, a hybridization-triggered cleavage agent, etc.

ORFX polypeptides

The novel protein of the invention includes the ORFX-like protein whose sequence is provided in any of SEQ ID NO: 2n (wherein n=1 to 132). The invention also includes a mutant or variant protein any of whose residues may be changed from the corresponding residue shown in FIG. 1 while still encoding a protein that maintains its ORFX-like activities and physiological functions, or a functional fragment thereof. For example, the invention includes the polypeptides encoded by the variant ORFX nucleic acids described above. In the mutant or variant protein, up to 20% or more of the residues may be so changed.

In general, an ORFX-like variant that preserves ORFX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above. Furthermore, without limiting the scope of the invention, positions of any of SEQ ID NO: 2n (wherein n=1 to 132) may be substitute such that a mutant or variant protein may include one or more substitutions

The invention also includes isolated ORFX proteins, and biologically active portions thereof, or derivatives, fragments, analogs or homologs thereof. Also provided are polypeptide fragments suitable for use as immunogens to raise anti-ORFX antibodies. In one embodiment, native ORFX proteins can be isolated from cells or tissue sources by an appropriate purification scheme using standard protein purification techniques. In another embodiment, ORFX proteins are produced by recombinant DNA techniques. Alternative to recombinant expression, a ORFX protein or polypeptide can be synthesized chemically using standard peptide synthesis techniques.

An “isolated” or “purified” protein or biologically active portion thereof is substantially free of cellular material or other contaminating proteins from the cell or tissue source from which the ORFX protein is derived, or substantially free from chemical precursors or other chemicals when chemically synthesized. The language “substantially free of cellular material” includes preparations of ORFX protein in which the protein is separated from cellular components of the cells from which it is isolated or recombinantly produced. In one embodiment, the language “substantially free of cellular material” includes preparations of ORFX protein having less than about 30% (by dry weight) of non-ORFX protein (also referred to herein as a “contaminating protein”), more preferably less than about 20% of non-ORFX protein, still more preferably less than about 10% of non-ORFX protein, and most preferably less than about 5% non-ORFX protein. When the ORFX protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of ORFX protein in which the protein is separated from chemical precursors or other chemicals that are involved in the synthesis of the protein. In one embodiment, the language “substantially free of chemical precursors or other chemicals” includes preparations of ORFX protein having less than about 30% (by dry weight) of chemical precursors or non-ORFX chemicals, more preferably less than about 20% chemical precursors or non-ORFX chemicals, still more preferably less than about 10% chemical precursors or non-ORFX chemicals, and most preferably less than about 5% chemical precursors or non-ORFX chemicals.

Biologically active portions of a ORFX protein include peptides comprising amino acid sequences sufficiently homologous to or derived from the amino acid sequence of the ORFX protein, e.g., the amino acid sequence shown in SEQ ID NO: 2 that include fewer amino acids than the full length ORFX proteins, and exhibit at least one activity of a ORFX protein. Typically, biologically active portions comprise a domain or motif with at least one activity of the ORFX protein. A biologically active portion of a ORFX protein can be a polypeptide which is, for example, 10, 25, 50, 100 or more amino acids in length.

A biologically active portion of a ORFX protein of the present invention may contain at least one of the above-identified domains conserved between the FGF family of proteins. Moreover, other biologically active portions, in which other regions of the protein are deleted, can be prepared by recombinant techniques and evaluated for one or more of the functional activities of a native ORFX protein.

In an embodiment, the ORFX protein has an amino acid sequence shown in any of SEQ ID NO: 2n (wherein n=1 to 132). In other embodiments, the ORFX protein is substantially homologous to any of SEQ ID NO: 2n (wherein n=1 to 132) and retains the functional activity of the protein of any of SEQ ID NO: 2n (wherein n=1 to 132), yet differs in amino acid sequence due to natural allelic variation or mutagenesis, as described in detail below. Accordingly, in another embodiment, the ORFX protein is a protein that comprises an amino acid sequence at least about 45% homologous, and more preferably about 55, 65, 70, 75, 80, 85, 90, 95, 98 or even 99% homologous to the amino acid sequence of any of SEQ ID NO: 2n (wherein n=1 to 132) and retains the functional activity of the ORFX proteins of the corresponding polypeptide having the sequence of SEQ ID NO: 2n (wherein n=1 to 132).

Determining Homology between two or more Sequences

To determine the percent homology of two amino acid sequences or of two nucleic acids, the sequences are aligned for optimal comparison purposes (e.g., gaps can be introduced in either of the sequences being compared for optimal alignment between the sequences). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NO: 2n-1 (wherein n=1 to 132).

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. The term “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

Chimeric and Fusion Proteins

The invention also provides ORFX chimeric or fusion proteins. As used herein, a ORFX “chimeric protein” or “fusion protein” includes a ORFX polypeptide operatively linked to a non-ORFX polypeptide. A “ORFX polypeptide” refers to a polypeptide having an amino acid sequence corresponding to ORFX, whereas a “non-ORFX polypeptide” refers to a polypeptide having an amino acid sequence corresponding to a protein that is not substantially homologous to the ORFX protein, e.g., a protein that is different from the ORFX protein and that is derived from the same or a different organism. Within a ORFX fusion protein the ORFX polypeptide can correspond to all or a portion of a ORFX protein. In one embodiment, a ORFX fusion protein comprises at least one biologically active portion of a ORFX protein. In another embodiment, a ORFX fusion protein comprises at least two biologically active portions of a ORFX protein. Within the fusion protein, the term “operatively linked” is intended to indicate that the ORFX polypeptide and the non-ORFX polypeptide are fused in-frame to each other. The non-ORFX polypeptide can be fused to the N-terminus or C-terminus of the ORFX polypeptide.

For example, in one embodiment a ORFX fusion protein comprises a ORFX polypeptide operably linked to the extracellular domain of a second protein. Such fusion proteins can be further utilized in screening assays for compounds that modulate ORFX activity (such assays are described in detail below).

In another embodiment, the fusion protein is a GST-ORFX fusion protein in which the ORFX sequences are fused to the C-terminus of the GST (i.e., glutathione S-transferase) sequences. Such fusion proteins can facilitate the purification of recombinant ORFX.

In yet another embodiment, the fusion protein is a ORFX protein containing a heterologous signal sequence at its N-terminus. For example, the native ORFX signal sequence can be removed and replaced with a signal sequence from another protein. In certain host cells (e.g., mammalian host cells), expression and/or secretion of ORFX can be increased through use of a heterologous signal sequence.

In another embodiment, the fusion protein is a ORFX-immunoglobulin fusion protein in which the ORFX sequences comprising one or more domains are fused to sequences derived from a member of the immunoglobulin protein family. The ORFX-immunoglobulin fusion proteins of the invention can be incorporated into pharmaceutical compositions and administered to a subject to inhibit an interaction between a ORFX ligand and a ORFX protein on the surface of a cell, to thereby suppress ORFX-mediated signal transduction in vivo. In one nonlimiting example, a contemplated ORFX ligand of the invention is an ORFX receptor. The ORFX-immunoglobulin fusion proteins can be used to modulate the bioavailability of a ORFX cognate ligand. Inhibition of the ORFX ligand/ORFX interaction may be useful therapeutically for both the treatment of proliferative and differentiative disorders, as well as modulating (e.g., promoting or inhibiting) cell survival. Moreover, the ORFX-immunoglobulin fusion proteins of the invention can be used as immunogens to produce anti-ORFX antibodies in a subject, to purify ORFX ligands, and in screening assays to identify molecules that inhibit the interaction of ORFX with a ORFX ligand.

A ORFX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A ORFX-encoding nucleic acid can be cloned into such an expression vector such that the fusion moiety is linked in-frame to the ORFX protein.

ORFX Agonists and Antagonists

The present invention also pertains to variants of the ORFX proteins that function as either ORFX agonists (mimetics) or as ORFX antagonists. Variants of the ORFX protein can be generated by mutagenesis, e.g., discrete point mutation or truncation of the ORFX protein. An agonist of the ORFX protein can retain substantially the same, or a subset of, the biological activities of the naturally occurring form of the ORFX protein. An antagonist of the ORFX protein can inhibit one or more of the activities of the naturally occurring form of the ORFX protein by, for example, competitively binding to a downstream or upstream member of a cellular signaling cascade which includes the ORFX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the ORFX proteins.

Variants of the ORFX protein that function as either ORFX agonists (mimetics) or as ORFX antagonists can be identified by screening combinatorial libraries of mutants, e.g., truncation mutants, of the ORFX protein for ORFX protein agonist or antagonist activity. In one embodiment, a variegated library of ORFX variants is generated by combinatorial mutagenesis at the nucleic acid level and is encoded by a variegated gene library. A variegated library of ORFX variants can be produced by, for example, enzymatically ligating a mixture of synthetic oligonucleotides into gene sequences such that a degenerate set of potential ORFX sequences is expressible as individual polypeptides, or alternatively, as a set of larger fusion proteins (e.g., for phage display) containing the set of ORFX sequences therein. There are a variety of methods which can be used to produce libraries of potential ORFX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential ORFX sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl Acid Res 11:477.

Polypeptide Libraries

In addition, libraries of fragments of the ORFX protein coding sequence can be used to generate a variegated population of ORFX fragments for screening and subsequent selection of variants of a ORFX protein. In one embodiment, a library of coding sequence fragments can be generated by treating a double stranded PCR fragment of a ORFX coding sequence with a nuclease under conditions wherein nicking occurs only about once per molecule, denaturing the double stranded DNA, renaturing the DNA to form double stranded DNA that can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the ORFX protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of ORFX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique that enhances the frequency of functional mutants in the libraries, can be used in combination with the screening assays to identify ORFX variants (Arkin and Yourvan (1992) PNAS 89:7811-7815; Delgrave et al. (1993) Protein Engineering 6:327-331).

Anti-oRFX Antibodies

The invention further encompasses antibodies and antibody fragments, such as F_{ab} or (F_{ab)2}, that bind immunospecifically to any of the proteins of the invention.

An isolated ORFX protein, or a portion or fragment thereof, can be used as an immunogen to generate antibodies that bind ORFX using standard techniques for polyclonal and monoclonal antibody preparation. Full-length ORFX protein can be used. Alternatively, the invention provides antigenic peptide fragments of ORFX for use as immunogens. The antigenic peptide of ORFX comprises at least 4 amino acid residues of the amino acid sequence shown in any of SEQ ID NO: 2n (wherein n=1 to 132). The antigenic peptide encompasses an epitope of ORFX such that an antibody raised against the peptide forms a specific immune complex with ORFX. The antigenic peptide may comprise at least 6 aa residues, at least 8 aa residues, at least 10 aa residues, at least 15 aa residues, at least 20 aa residues, or at least 30 aa residues. In one embodiment of the invention, the antigenic peptide comprises a polypeptide comprising at least 6 contiguous amino acids of any of SEQ ID NO: 2n (wherein n=1 to 132).

In an embodiment of the invention, epitopes encompassed by the antigenic peptide are regions of ORFX that are located on the surface of the protein, e.g., hydrophilic regions. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety.

As disclosed herein, an ORFX protein sequence of any of SEQ ID NO: 2n (wherein n=1 to 132), or derivatives, fragments, analogs or homologs thereof, may be utilized as immunogens in the generation of antibodies that immunospecifically-bind these protein components. The term “antibody” as used herein refers to immunoglobulin molecules and immunologically active portions of immunoglobulin molecules, i.e., molecules that contain an antigen binding site that specifically binds (immunoreacts with) an antigen, such as ORFX. Such antibodies include, but are not limited to, polyclonal, monoclonal, chimeric, single chain, F_{ab} and F(_{ab′)2} fragments, and an F_{ab} expression library. In a specific embodiment, antibodies to human ORFX proteins are disclosed. Various procedures known within the art may be used for the production of polyclonal or monoclonal antibodies to a ORFX protein sequence of any of SEQ ID NO: 2n (wherein n=1 to 132) or derivative, fragment, analog or homolog thereof. Some of these proteins are discussed below.

For the production of polyclonal antibodies, various suitable host animals (e.g., rabbit, goat, mouse or other mammal) may be immunized by injection with the native protein, or a synthetic variant thereof, or a derivative of the foregoing. An appropriate immunogenic preparation can contain, for example, recombinantly expressed ORFX protein or a chemically synthesized ORFX polypeptide. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against ORFX can be isolated from the mammal (e.g., from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of ORFX. A monoclonal antibody composition thus typically displays a single binding affinity for a particular ORFX protein with which it immunoreacts. For preparation of monoclonal antibodies directed towards a particular ORFX protein, or derivatives, fragments, analogs or homologs thereof, any technique that provides for the production of antibody molecules by continuous cell line culture may be utilized. Such techniques include, but are not limited to, the hybridoma technique (see Kohler & Milstein, 1975 Nature 256: 495-497); the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983, Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96). Each of the above citations are incorporated herein by reference in their entirety

According to the invention, techniques can be adapted for the production of single-chain antibodies specific to a ORFX protein (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a ORFX protein or derivatives, fragments, analogs or homologs thereof. Non-human antibodies can be “humanized” by techniques well known in the art. See e.g., U.S. Pat. No. 5,225,539. Each of the above citations are incorporated herein by reference. Antibody fragments that contain the idiotypes to a ORFX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Additionally, recombinant anti-ORFX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT International Application No. PCT/US 86/02269; European Patent Application No. 184,187; European Patent Application No. 171,496; European Patent Application No. 173,494; PCT International Publication No. WO 86/01533; U.S. Pat. No. 4,816,567; European Patent Application No. 125,023; Better et al.(1988) Science 240:1041-1043; Liu et al. (1987) PNAS 84:3439-3443; Liu et al. (1987) J Immunol. 139:3521-3526; Sun et al. (1987) PNAS 84:214-218; Nishimura et al. (1987) Cancer Res 47:999-1005; Wood et al. (1985)Nature 314:446-449; Shaw et al. (1988), J. Natl Cancer Inst 80:1553-1559); Morrison(1985) Science 229:1202-1207; Oi et al. (1986) BioTechniques 4:214; U.S. Pat. No. 5,225,539; Jones et al. (1986) Nature 321:552-525; Verhoeyan et al. (1988) Science 239:1534; and Beidler et al. (1988) J Immunol 141:4053-4060 . Each of the above citations are incorporated herein by reference.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of a ORFX protein is facilitated by generation of hybridomas that bind to the fragment of a ORFX protein possessing such a domain. Antibodies that are specific for one or more domains within a ORFX protein, e.g., the domain spanning the first fifty amino-terminal residues specific to ORFX when compared to FGF-9, or derivatives, fragments, analogs or homologs thereof, are also provided herein.

Anti-ORFX antibodies may be used in methods known within the art relating to the localization and/or quantitation of a ORFX protein (e.g., for use in measuring levels of the ORFX protein within appropriate physiological samples, for use in diagnostic methods, for use in imaging the protein, and the like). In a given embodiment, antibodies for ORFX proteins, or derivatives, fragments, analogs or homologs thereof, that contain the antibody derived binding domain, are utilized as pharmacologically-active compounds [hereinafter “Therapeutics”].

An anti-ORFX antibody (e.g., monoclonal antibody) can be used to isolate ORFX by standard techniques, such as affinity chromatography or immunoprecipitation. An anti-ORFX antibody can facilitate the purification of natural ORFX from cells and of recombinantly produced ORFX expressed in host cells. Moreover, an anti-ORFX antibody can be used to detect ORFX protein (e.g., in a cellular lysate or cell supernatant) in order to evaluate the abundance and pattern of expression of the ORFX protein. Anti-ORFX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

ORFX Recombinant Vectors and Host Cells

Another aspect of the invention pertains to vectors, preferably expression vectors, containing a nucleic acid encoding ORFX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., ORFX proteins, mutant forms of ORFX, fusion proteins, etc.).

The recombinant expression vectors of the invention can be designed for expression of ORFX in prokaryotic or eukaryotic cells. For example, ORFX can be expressed in bacterial cells such as E. coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 1 id (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:2111-2118). Such alteration of nucleic acid sequences of the invention can be carried out by standard DNA synthesis techniques.

In another embodiment, the ORFX expression vector is a yeast expression vector. Examples of vectors for expression in yeast S. cerivisae include pYepSec1 (Baldari, et al., (1987) EMBO J 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, ORFX can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells. See, e.g., Chapters 16 and 17 of Sambrook et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev 1:268-277), lymphoid-specific promoters (Calame and Eaton (1988) Adv Immunol 43:235-275), in particular promoters of T cell receptors (Winoto and Baltimore (1989) EMBO J 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) PNAS 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss (1990) Science 249:374-379) and the α-fetoprotein promoter (Campes and Tilghman (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to ORFX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al., “Antisense RNA as a molecular tool for genetic analysis,” Reviews——Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, ORFX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding ORFX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) ORFX protein. Accordingly, the invention further provides methods for producing ORFX protein using the host cells of the invention. In one embodiment, the method comprises culturing the host cell of invention (into which a recombinant expression vector encoding ORFX has been introduced) in a suitable medium such that ORFX protein is produced. In another embodiment, the method further comprises isolating ORFX from the medium or the host cell.

Transgenic Animals

The host cells of the invention can also be used to produce nonhuman transgenic animals. For example, in one embodiment, a host cell of the invention is a fertilized oocyte or an embryonic stem cell into which ORFX-coding sequences have been introduced. Such host cells can then be used to create non-human transgenic animals in which exogenous ORFX sequences have been introduced into their genome or homologous recombinant animals in which endogenous ORFX sequences have been altered. Such animals are useful for studying the function and/or activity of ORFX and for identifying and/or evaluating modulators of ORFX activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous ORFX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing ORFX-encoding nucleic acid into the male pronuclei of a fertilized oocyte, e.g., by microinjection, retroviral infection, and allowing the oocyte to develop in a pseudopregnant female foster animal. The human ORFX DNA sequence of SEQ ID NO: 2n-1 (wherein n=1 to 132) can be introduced as a transgene into the genome of a non-human animal. Alternatively, a nonhuman homologue of the human ORFX gene, such as a mouse ORFX gene, can be isolated based on hybridization to the human ORFX cDNA (described further above) and used as a transgene. Intronic sequences and polyadenylation signals can also be included in the transgene to increase the efficiency of expression of the transgene. A tissue-specific regulatory sequence(s) can be operably linked to the ORFX transgene to direct expression of ORFX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan 1986, In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the ORFX transgene in its genome and/or expression of ORFX mRNA in tissues or cells of the animals. A transgenic founder animal can then be used to breed additional animals carrying the transgene. Moreover, transgenic animals carrying a transgene encoding ORFX can further be bred to other transgenic animals carrying other transgenes.

To create a homologous recombinant animal, a vector is prepared which contains at least a portion of a ORFX gene into which a deletion, addition or substitution has been introduced to thereby alter, e.g., functionally disrupt, the ORFX gene. The ORFX gene can be a human gene (e.g., SEQ ID NO: 2n-1 (wherein n=1 to 132)), but more preferably, is a non-human homologue of a human ORFX gene. For example, a mouse homologue of human ORFX gene of SEQ ID NO: 2n-1 (wherein n=1 to 132) can be used to construct a homologous recombination vector suitable for altering an endogenous ORFX gene in the mouse genome. In one embodiment, the vector is designed such that, upon homologous recombination, the endogenous ORFX gene is functionally disrupted (i.e., no longer encodes a functional protein; also referred to as a “knock out” vector).

Alternatively, the vector can be designed such that, upon homologous recombination, the endogenous ORFX gene is mutated or otherwise altered but still encodes functional protein (e.g., the upstream regulatory region can be altered to thereby alter the expression of the endogenous ORFX protein). In the homologous recombination vector, the altered portion of the ORFX gene is flanked at its 5′ and 3′ ends by additional nucleic acid of the ORFX gene to allow for homologous recombination to occur between the exogenous ORFX gene carried by the vector and an endogenous ORFX gene in an embryonic stem cell. The additional flanking ORFX nucleic acid is of sufficient length for successful homologous recombination with the endogenous gene. Typically, several kilobases of flanking DNA (both at the 5′ and 3′ ends) are included in the vector. See e.g., Thomas et al. (1987)Cell 51:503 for a description of homologous recombination vectors. The vector is introduced into an embryonic stem cell line (e.g., by electroporation) and cells in which the introduced ORFX gene has homologously recombined with the endogenous ORFX gene are selected (see e.g., Li et al. (1992) Cell 69:915).

The selected cells are then injected into a blastocyst of an animal (e.g., a mouse) to form aggregation chimeras. See e.g., Bradley 1987, In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Curr Opin Biotechnol 2:823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813. In brief, a cell, e.g., a somatic cell, from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell, e.g., the somatic cell, is isolated.

Pharmaceutical Compositions

The ORFX nucleic acid molecules, ORFX proteins, and anti-ORFX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antiflngal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a ORFX protein or anti-ORFX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

The nucleic acid molecules of the invention can be inserted into vectors and used as gene therapy vectors. Gene therapy vectors can be delivered to a subject by any of a number of routes, e.g., as described in U.S. Patent Nos. 5,703,055. Delivery can thus also include, e.g., intravenous injection, local administration (see U.S. Pat. No. 5,328,470) or stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Additional Uses and Methods of the Invention

The nucleic acid molecules, proteins, protein homologues, and antibodies described herein can be used in one or more of the following methods: (a) screening assays; (b) detection assays (e.g., chromosomal mapping, cell and tissue typing, forensic biology), (c) predictive medicine (e.g., diagnostic assays, prognostic assays, monitoring clinical trials, and pharmacogenomics); and (d) methods of treatment (e.g., therapeutic and prophylactic).

The isolated nucleic acid molecules of the invention can be used to express ORFX protein (e.g., via a recombinant expression vector in a host cell in gene therapy applications), to detect ORFX mRNA (e.g., in a biological sample) or a genetic lesion in a ORFX gene, and to modulate ORFX activity, as described further below. In addition, the ORFX proteins can be used to screen drugs or compounds that modulate the ORFX activity or expression as well as to treat disorders characterized by insufficient or excessive production of ORFX protein, for example proliferative or differentiative disorders, or production of ORFX protein forms that have decreased or aberrant activity compared to ORFX wild type protein. In addition, the anti-ORFX antibodies of the invention can be used to detect and isolate ORFX proteins and modulate ORFX activity.

This invention further pertains to novel agents identified by the above described screening assays and uses thereof for treatments as described herein.

Screening Assays

The invention provides a method (also referred to herein as a “screening assay”) for identifying modulators, i.e., candidate or test compounds or agents (e.g., peptides, peptidomimetics, small molecules or other drugs) that bind to ORFX proteins or have a stimulatory or inhibitory effect on, for example, ORFX expression or ORFX activity.

In one embodiment, the invention provides assays for screening candidate or test compounds which bind to or modulate the activity of a ORFX protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckernann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of ORFX protein, or a biologically active portion thereof, on the cell surface is contacted with a test compound and the ability of the test compound to bind to a ORFX protein determined. The cell, for example, can of mammalian origin or a yeast cell. Determining the ability of the test compound to bind to the ORFX protein can be accomplished, for example, by coupling the test compound with a radioisotope or enzymatic label such that binding of the test compound to the ORFX protein or biologically active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of ORFX protein, or a biologically active portion thereof, on the cell surface with a known compound which binds ORFX to form an assay mixture, contacting the assay mixture with a test compound, and determining the ability of the test compound to interact with a ORFX protein, wherein determining the ability of the test compound to interact with a ORFX protein comprises determining the ability of the test compound to preferentially bind to ORFX or a biologically active portion thereof as compared to the known compound.

In another embodiment, an assay is a cell-based assay comprising contacting a cell expressing a membrane-bound form of ORFX protein, or a biologically active portion thereof, on the cell surface with a test compound and determining the ability of the test compound to modulate (e.g., stimulate or inhibit) the activity of the ORFX protein or biologically active portion thereof. Determining the ability of the test compound to modulate the activity of ORFX or a biologically active portion thereof can be accomplished, for example, by determining the ability of the ORFX protein to bind to or interact with a ORFX target molecule. As used herein, a “target molecule” is a molecule with which a ORFX protein binds or interacts in nature, for example, a molecule on the surface of a cell which expresses a ORFX interacting protein, a molecule on the surface of a second cell, a molecule in the extracellular milieu, a molecule associated with the internal surface of a cell membrane or a cytoplasmic molecule. A ORFX target molecule can be a non-ORFX molecule or a ORFX protein or polypeptide of the present invention. In one embodiment, a ORFX target molecule is a component of a signal transduction pathway that facilitates transduction of an extracellular signal (e.g., a signal generated by binding of a compound to a membrane-bound ORFX molecule) through the cell membrane and into the cell. The target, for example, can be a second intercellular protein that has catalytic activity or a protein that facilitates the association of downstream signaling molecules with ORFX.

Determining the ability of the ORFX protein to bind to or interact with a ORFX target molecule can be accomplished by one of the methods described above for determining direct binding. In one embodiment, determining the ability of the ORFX protein to bind to or interact with a ORFX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a ORFX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a ORFX protein or biologically active portion thereof with a test compound and determining the ability of the test compound to bind to the ORFX protein or biologically active portion thereof. Binding of the test compound to the ORFX protein can be determined either directly or indirectly as described above. In one embodiment, the assay comprises contacting the ORFX protein or biologically active portion thereof with a known compound which binds ORFX to form an assay mixture, contacting the assay mixture with a test compound, and determining the ability of the test compound to interact with a ORFX protein, wherein determining the ability of the test compound to interact with a ORFX protein comprises determining the ability of the test compound to preferentially bind to ORFX or biologically active portion thereof as compared to the known compound.

In another embodiment, an assay is a cell-free assay comprising contacting ORFX protein or biologically active portion thereof with a test compound and determining the ability of the test compound to modulate (e.g., stimulate or inhibit) the activity of the ORFX protein or biologically active portion thereof. Determining the ability of the test compound to modulate the activity of ORFX can be accomplished, for example, by determining the ability of the ORFX protein to bind to a ORFX target molecule by one of the methods described above for determining direct binding. In an alternative embodiment, determining the ability of the test compound to modulate the activity of ORFX can be accomplished by determining the ability of the ORFX protein further modulate a ORFX target molecule. For example, the catalytic/enzymatic activity of the target molecule on an appropriate substrate can be determined as previously described.

In yet another embodiment, the cell-free assay comprises contacting the ORFX protein or biologically active portion thereof with a known compound which binds ORFX to form an assay mixture, contacting the assay mixture with a test compound, and determining the ability of the test compound to interact with a ORFX protein, wherein determining the ability of the test compound to interact with a ORFX protein comprises determining the ability of the ORFX protein to preferentially bind to or modulate the activity of a ORFX target molecule.

The cell-free assays of the present invention are amenable to use of both the soluble form or the membrane-bound form of ORFX. In the case of cell-free assays comprising the membrane-bound form of ORFX, it may be desirable to utilize a solubilizing agent such that the membrane-bound form of ORFX is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either ORFX or its target molecule to facilitate separation of p complexed from uncomplexed forms of one or both of the proteins, as well as to accommodate automation of the assay. Binding of a test compound to ORFX, or interaction of ORFX with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-ORFX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or ORFX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of ORFX binding or activity determined using standard techniques.

Other techniques for immobilizing proteins on matrices can also be used in the screening assays of the invention. For example, either ORFX or its target molecule can be immobilized utilizing conjugation of biotin and streptavidin. Biotinylated ORFX or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with ORFX or target molecules, but which do not interfere with binding of the ORFX protein to its target molecule, can be derivatized to the wells of the plate, and unbound target or ORFX trapped in the wells by antibody conjugation. Methods for detecting such complexes, in addition to those described above for the GST-immobilized complexes, include immunodetection of complexes using antibodies reactive with the ORFX or target molecule, as well as enzyme-linked assays that rely on detecting an enzymatic activity associated with the ORFX or target molecule.

In another embodiment, modulators of ORFX expression are identified in a method wherein a cell is contacted with a candidate compound and the expression of ORFX mRNA or protein in the cell is determined. The level of expression of ORFX mRNA or protein in the presence of the candidate compound is compared to the level of expression of ORFX mRNA or protein in the absence of the candidate compound. The candidate compound can then be identified as a modulator of ORFX expression based on this comparison. For example, when expression of ORFX mRNA or protein is greater (statistically significantly greater) in the presence of the candidate compound than in its absence, the candidate compound is identified as a stimulator of ORFX mRNA or protein expression. Alternatively, when expression of ORFX mRNA or protein is less (statistically significantly less) in the presence of the candidate compound than in its absence, the candidate compound is identified as an inhibitor of ORFX mRNA or protein expression. The level of ORFX mRNA or protein expression in the cells can be determined by methods described herein for detecting ORFX mRNA or protein.

In yet another aspect of the invention, the ORFX proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with ORFX (“ORFX-binding proteins” or “ORFX-bp”) and modulate ORFX activity. Such ORFX-binding proteins are also likely to be involved in the propagation of signals by the ORFX proteins as, for example, upstream or downstream elements of the ORFX pathway.

The two-hybrid system is based on the modular nature of most transcription factors, which consist of separable DNA-binding and activation domains. Briefly, the assay utilizes two different DNA constructs. In one construct, the gene that codes for ORFX is fused to a gene encoding the DNA binding domain of a known transcription factor (e.g., GAL-4). In the other construct, a DNA sequence, from a library of DNA sequences, that encodes an unidentified protein (“prey” or “sample” is fused to a gene that codes for the activation domain of the known transcription factor. If the “bait” and the “prey” proteins are able to interact, in vivo, forming a ORFX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with ORFX.

This invention further pertains to novel agents identified by the above-described screening assays and uses thereof for treatments as described herein.

Detection Assays

Portions or fragments of the cDNA sequences identified herein (and the corresponding complete gene sequences) can be used in numerous ways as polynucleotide reagents. For example, these sequences can be used to: (i) map their respective genes on a chromosome; and, thus, locate gene regions associated with genetic disease; (ii) identify an individual from a minute biological sample (tissue typing); and (iii) aid in forensic identification of a biological sample.

The ORFX sequences of the present invention can also be used to identify individuals from minute biological samples. In this technique, an individual's genomic DNA is digested with one or more restriction enzymes, and probed on a Southern blot to yield unique bands for identification. The sequences of the present invention are useful as additional DNA markers for RFLP (“restriction fragment length polymorphisms,” described in U.S. Pat. No. 5,272,057).

Furthermore, the sequences of the present invention can be used to provide an alternative technique that determines the actual base-by-base DNA sequence of selected portions of an individual's genome. Thus, the ORFX sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The ORFX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 2n-1 (wherein n=1 to 132), as described above, can comfortably provide positive individual identification with a panel of perhaps 10 to 1,000 primers that each yield a noncoding amplified sequence of 100 bases. If predicted coding sequences are used, a more appropriate number of primers for positive individual identification would be 500-2,000.

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining ORFX protein and/or nucleic acid expression as well as ORFX activity, in the context of a biological sample (e.g., blood, serum, cells, tissue) to thereby determine whether an individual is afflicted with a disease or disorder, or is at risk of developing a disorder, associated with aberrant ORFX expression or activity. The invention also provides for prognostic (or predictive) assays for determining whether an individual is at risk of developing a disorder associated with ORFX protein, nucleic acid expression or activity. For example, mutations in a ORFX gene can be assayed in a biological sample. Such assays can be used for prognostic or predictive purpose to thereby prophylactically treat an individual prior to the onset of a disorder characterized by or associated with ORFX protein, nucleic acid expression or activity.

Another aspect of the invention provides methods for determining ORFX protein, nucleic acid expression or ORFX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of ORFX in clinical trials.

Use of Partial ORFX Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, that can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 2n-1 (where n=1 to 132) are particularly appropriate for this use as greater numbers of polymorphisms occur in the noncoding regions, making it easier to differentiate individuals using this technique. Examples of polynucleotide reagents include the ORFX sequences or portions thereof, e.g., fragments derived from the noncoding regions of one or more of SEQ ID NO: 2n-1 (where n=1 to 132), having a length of at least 20 bases, preferably at least 30 bases.

The ORFX sequences described herein can further be used to provide polynucleotide reagents, e.g., labeled or label-able probes that can be used, for example, in an in situ hybridization technique, to identify a specific tissue, e.g., brain tissue, etc. This can be very useful in cases where a forensic pathologist is presented with a tissue of unknown origin. Panels of such ORFX probes can be used to identify tissue by species and/or by organ type.

In a similar fashion, these reagents, e.g., ORFX primers or probes can be used to screen tissue culture for contamination (i.e. screen for the presence of a mixture of different types of cells in a culture).

Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining ORFX protein and/or nucleic acid expression as well as ORFX activity, in the context of a biological sample (e.g., blood, serum, cells, tissue) to thereby determine whether an individual is afflicted with a disease or disorder, or is at risk of developing a disorder, associated with aberrant ORFX expression or activity. The invention also provides for prognostic (or predictive) assays for determining whether an individual is at risk of developing a disorder associated with ORFX protein, nucleic acid expression or activity. For example, mutations in a ORFX gene can be assayed in a biological sample. Such assays can be used for prognostic or predictive purpose to thereby prophylactically treat an individual prior to the onset of a disorder characterized by or associated with ORFX protein, nucleic acid expression or activity.

Another aspect of the invention provides methods for determining ORFX protein, nucleic acid expression or ORFX activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of ORFX in clinical trials.

These and other agents are described in further detail in the following sections.

Diagnostic Assays

Other conditions in which proliferation of cells plays a role include tumors, restenosis, psoriasis, Dupuytren's contracture, diabetic complications, Kaposi's sarcoma and rheumatoid arthritis.

An ORFX polypeptide may be used to identify an interacting polypeptide a sample or tissue. The method comprises contacting the sample or tissue with ORFX, allowing formation of a complex between the ORFX polypeptide and the interacting polypeptide, and detecting the complex, if present.

The proteins of the invention may be used to stimulate production of antibodies specifically binding the proteins. Such antibodies may be used in immunodiagnostic procedures to detect the occurrence of the protein in a sample. The proteins of the invention may be used to stimulate cell growth and cell proliferation in conditions in which such growth would be favorable. An example would be to counteract toxic side effects of chemotherapeutic agents on, for example, hematopoiesis and platelet formation, linings of the gastrointestinal tract, and hair follicles. They may also be used to stimulate new cell growth in neurological disorders including, for example, Alzheimer's disease. Alternatively, antagonistic treatments may be administered in which an antibody specifically binding the ORFX-like proteins of the invention would abrogate the specific growth-inducing effects of the proteins. Such antibodies may be useful, for example, in the treatment of proliferative disorders including various tumors and benign hyperplasias.

Polynucleotides or oligonucleotides corresponding to any one portion of the ORFX nucleic acids of SEQ ID NO: 2n-1 (wherein n=1 to 132) may be used to detect DNA containing a corresponding ORF gene, or detect the expression of a corresponding ORFX gene, or ORFX-like gene. For example, an ORFX nucleic acid expressed in a particular cell or tissue, as noted in Table 2, can be used to identify the presence of that particular cell type.

An exemplary method for detecting the presence or absence of ORFX in a biological sample involves obtaining a biological sample from a test subject and contacting the biological sample with a compound or an agent capable of detecting ORFX protein or nucleic acid (e.g., mRNA, genomic DNA) that encodes ORFX protein such that the presence of ORFX is detected in the biological sample. An agent for detecting ORFX mRNA or genomic DNA is a labeled nucleic acid probe capable of hybridizing to ORFX mRNA or genomic DNA. The nucleic acid probe can be, for example, a full-length ORFX nucleic acid, such as the nucleic acid of SEQ ID NO: 2n-1 (wherein n=1 to 132), or a portion thereof, such as an oligonucleotide of at least 15, 30, 50, 100, 250 or 500 nucleotides in length and sufficient to specifically hybridize under stringent conditions to ORFX mRNA or genomic DNA, as described above. Other suitable probes for use in the diagnostic assays of the invention are described herein.

An agent for detecting ORFX protein is an antibody capable of binding to ORFX protein, preferably an antibody with a detectable label. Antibodies can be polyclonal, or more preferably, monoclonal. An intact antibody, or a fragment thereof (e.g., Fab or F(ab′ )₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect ORFX mRNA, protein, or genomic DNA in a biological sample in vitro as well as in vivo. For example, in vitro techniques for detection of ORFX mRNA include Northern hybridizations and in situ hybridizations. In vitro techniques for detection of ORFX protein include enzyme linked immunosorbent assays (ELISAs), Western blots, immunoprecipitations and immunofluorescence. In vitro techniques for detection of ORFX genomic DNA include Southern hybridizations. Furthermore, in vivo techniques for detection of ORFX protein include introducing into a subject a labeled anti-ORFX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting ORFX protein, mRNA, or genomic DNA, such that the presence of ORFX protein, mRNA or genomic DNA is detected in the biological sample, and comparing the presence of ORFX protein, mRNA or genomic DNA in the control sample with the presence of ORFX protein, mRNA or genomic DNA in the test sample.

The invention also encompasses kits for detecting the presence of ORFX in a biological sample. For example, the kit can comprise: a labeled compound or agent capable of detecting ORFX protein or mRNA in a biological sample; means for determining the amount of ORFX in the sample; and means for comparing the amount of ORFX in the sample with a standard. The compound or agent can be packaged in a suitable container. The kit can further comprise instructions for using the kit to detect ORFX protein or nucleic acid.

Prognostic Assays

The diagnostic methods described herein can furthermore be utilized to identify subjects having or at risk of developing a disease or disorder associated with aberrant ORFX expression or activity. For example, the assays described herein, such as the preceding diagnostic assays or the following assays, can be utilized to identify a subject having or at risk of developing a disorder associated with ORFX protein, nucleic acid expression or activity in, e.g., proliferative or differentiative disorders such as hyperplasias, tumors, restenosis, psoriasis, Dupuytren's contracture, diabetic complications, or rheumatoid arthritis, etc.; and glia-associated disorders such as cerebral lesions, diabetic neuropathies, cerebral edema, senile dementia, Alzheimer's disease, etc. Alternatively, the prognostic assays can be utilized to identify a subject having or at risk for developing a disease or disorder. Thus, the present invention provides a method for identifying a disease or disorder associated with aberrant ORFX expression or activity in which a test sample is obtained from a subject and ORFX protein or nucleic acid (e.g., mRNA, genomic DNA) is detected, wherein the presence of ORFX protein or nucleic acid is diagnostic for a subject having or at risk of developing a disease or disorder associated with aberrant ORFX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant ORFX expression or activity. For example, such methods can be used to determine whether a subject can be effectively treated with an agent for a disorder, such as a proliferative disorder, differentiative disorder, glia-associated disorders, etc. Thus, the present invention provides methods for determining whether a subject can be effectively treated with an agent for a disorder associated with aberrant ORFX expression or activity in which a test sample is obtained and ORFX protein or nucleic acid is detected (e.g., wherein the presence of ORFX protein or nucleic acid is diagnostic for a subject that can be administered the agent to treat a disorder associated with aberrant ORFX expression or activity.)

The methods of the invention can also be used to detect genetic lesions in a ORFX gene, thereby determining if a subject with the lesioned gene is at risk for, or suffers from, a proliferative disorder, differentiative disorder, glia-associated disorder, etc. In various embodiments, the methods include detecting, in a sample of cells from the subject, the presence or absence of a genetic lesion characterized by at least one of an alteration affecting the integrity of a gene encoding a ORFX-protein, or the mis-expression of the ORFX gene. For example, such genetic lesions can be detected by ascertaining the existence of at least one of (1) a deletion of one or more nucleotides from a ORFX gene; (2) an addition of one or more nucleotides to a ORFX gene; (3) a substitution of one or more nucleotides of a ORFX gene, (4) a chromosomal rearrangement of a ORFX gene; (5) an alteration in the level of a messenger RNA transcript of a ORFX gene, (6) aberrant modification of a ORFX gene, such as of the methylation pattern of the genomic DNA, (7) the presence of a non-wild type splicing pattern of a messenger RNA transcript of a ORFX gene, (8) a non-wild type level of a ORFX-protein, (9) allelic loss of a ORFX gene, and (10) inappropriate post-translational modification of a ORFX-protein. As described herein, there are a large number of assay techniques known in the art which can be used for detecting lesions in a ORFX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the ORFX-gene (see Abravaya et al. (1995) Nucl Acids Res 23:675-682). This method can include the steps of collecting a sample of cells from a patient, isolating nucleic acid (e.g., genomic, mRNA or both) from the cells of the sample, contacting the nucleic acid sample with one or more primers that specifically hybridize to a ORFX gene under conditions such that hybridization and amplification of the ORFX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al., 1990, Proc Natl Acad Sci USA 87:1874-1878), transcriptional amplification system (Kwoh, et al., 1989, Proc Natl Acad Sci USA 86:1173-1177), Q-Beta Replicase (Lizardi et al, 1988, BioTechnology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a ORFX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,493,531) can be used to score for the presence of specific mutations by development or loss of a ribozyme cleavage site.

In other embodiments, genetic mutations in ORFX can be identified by hybridizing a sample and control nucleic acids, e.g., DNA or RNA, to high density arrays containing hundreds or thousands of oligonucleotides probes (Cronin et al. (1996) Human Mutation 7: 244-255; Kozal et al. (1996) Nature Medicine 2: 753-759). For example, genetic mutations in ORFX can be identified in two dimensional arrays containing light-generated DNA probes as described in Cronin et al. above. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the ORFX gene and detect mutations by comparing the sequence of the sample ORFX with the corresponding wild-type (control) sequence. Examples of sequencing reactions include those based on techniques developed by Maxim and Gilbert (1977) PNAS 74:560 or Sanger (1977) PNAS 74:5463. It is also contemplated that any of a variety of automated sequencing procedures can be utilized when performing the diagnostic assays (Naeve et al., (1995) Biotechniques 19:448), including sequencing by mass spectrometry (see, e.g., PCT International Publ. No. WO 94/16101; Cohen et al. (1996) Adv Chromatogr 36:127-162; and Griffin et al. (1993) Appl Biochem Biotechnol 38:147-159).

Other methods for detecting mutations in the ORFX gene include methods in which protection from cleavage agents is used to detect mismatched bases in RNA/RNA or RNA/DNA heteroduplexes (Myers et al. (1985) Science 230:1242). In general, the art technique of “mismatch cleavage” starts by providing heteroduplexes of formed by hybridizing (labeled) RNA or DNA containing the wild-type ORFX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc Natl Acad Sci USA 85:4397; Saleeba et al (1992) Methods Enzymol 217:286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in ORFX cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a ORFX sequence, e.g., a wild-type ORFX sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify mutations in ORFX genes. For example, single strand conformation polymorphism (SSCP) may be used to detect differences in electrophoretic mobility between mutant and wild type nucleic acids (Orita et al. (1989) Proc Natl Acad Sci USA: 86:2766, see also Cotton (1993) Mutat Res 285:125-144; Hayashi (1992) Genet Anal Tech Appl 9:73-79). Single-stranded DNA fragments of sample and control ORFX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA, rather than DNA, in which the secondary structure is more sensitive to a change in sequence. In one embodiment, the subject method utilizes heteroduplex analysis to separate double stranded heteroduplex molecules on the basis of changes in electrophoretic mobility. See, e.g., Keen et al. (1991) Trends Genet 7:5.

In yet another embodiment the movement of mutant or wild-type fragments in polyacrylamide gels containing a gradient of denaturant is assayed using denaturing gradient gel electrophoresis (DGGE). See, e.g., Myers et al (1985) Nature 313:495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner (1987) Biophys Chem 265:12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki et al. (1986) Nature 324:163); Saiki et al. (1989) Proc Natl Acad. Sci USA 86:6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res 17:2437-2448) or at the extreme 3′ end of one primer where, under appropriate conditions, mismatch can prevent, or reduce polymerase extension (Prossner (1993) Tibtech 11:238). In addition it may be desirable to introduce a novel restriction site in the region of the mutation to create cleavage-based detection. See, e.g., Gasparini et al (1992) Mol Cell Probes 6:1. It is anticipated that in certain embodiments amplification may also be performed using Taq ligase for amplification. See, e.g., Barany (1991) Proc Natl Acad Sci USA 88:189. In such cases, ligation will occur only if there is a perfect match at the 3′ end of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a ORFX gene.

Furthermore, any cell type or tissue, preferably peripheral blood leukocytes, in which ORFX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on ORFX activity (e.g., ORFX gene expression), as identified by a screening assay described herein can be administered to individuals to treat (prophylactically or therapeutically) disorders (e.g., neurological, cancer-related or gestational disorders) associated with aberrant ORFX activity. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ORFX protein, expression of ORFX nucleic acid, or mutation content of ORFX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996, Clin Exp Pharmacol Physiol, 23:983-985 and Linder, 1997, Clin Chem, 43:254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of ORFX protein, expression of ORFX nucleic acid, or mutation content of ORFX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a ORFX modulator, such as a modulator identified by one of the exemplary screening assays described herein.

Monitoring Clinical Efficacy

Monitoring the influence of agents (e.g., drugs, compounds) on the expression or activity of ORFX (e.g. the ability to modulate aberrant cell proliferation and/or differentiation) can be applied in basic drug screening and in clinical trials. For example, the effectiveness of an agent determined by a screening assay as described herein to increase ORFX gene expression, protein levels, or upregulate ORFX activity, can be monitored in clinical trials of subjects exhibiting decreased ORFX gene expression, protein levels, or downregulated ORFX activity. Alternatively, the effectiveness of an agent determined by a screening assay to decrease ORFX gene expression, protein levels, or downregulate ORFX activity, can be monitored in clinical trials of subjects exhibiting increased ORFX gene expression, protein levels, or upregulated ORFX activity. In such clinical trials, the expression or activity of ORFX and, preferably, other genes that have been implicated in, for example, a proliferative or neurological disorder, can be used as a “read out” or marker of the responsiveness of a particular cell.

For example, genes, including ORFX, that are modulated in cells by treatment with an agent (e.g., compound, drug or small molecule) that modulates ORFX activity (e.g., identified in a screening assay as described herein) can be identified. Thus, to study the effect of agents on cellular proliferation disorders, for example, in a clinical trial, cells can be isolated and RNA prepared and analyzed for the levels of expression of ORFX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of ORFX or other genes. In this way, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, nucleic acid, peptidomimetic, small molecule, or other drug candidate identified by the screening assays described herein) comprising the steps of (i) obtaining a pre-administration sample from a subject prior to administration of the agent; (ii) detecting the level of expression of a ORFX protein, mRNA, or genomic DNA in the preadministration sample; (iii) obtaining one or more post-administration samples from the subject; (iv) detecting the level of expression or activity of the ORFX protein, mRNA, or genomic DNA in the post-administration samples; (v) comparing the level of expression or activity of the ORFX protein, mRNA, or genomic DNA in the pre-administration sample with the ORFX protein, mRNA, or genomic DNA in the post administration sample or samples; and (vi) altering the administration of the agent to the subject accordingly. For example, increased administration of the agent may be desirable to increase the expression or activity of ORFX to higher levels than detected, i.e., to increase the effectiveness of the agent. Alternatively, decreased administration of the agent may be desirable to decrease expression or activity of ORFX to lower levels than detected, i.e., to decrease the effectiveness of the agent.

Methods of Treatment

The present invention provides for both prophylactic and therapeutic methods of treating a subject at risk of (or susceptible to) a disorder or having a disorder associated with aberrant ORFX expression or activity.

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to, (i) a ORFX polypeptide, or analogs, derivatives, fragments or homologs thereof; (ii) antibodies to a ORFX peptide; (iii) nucleic acids encoding a ORFX peptide; (iv) administration of antisense nucleic acid and nucleic acids that are “dysfunctional” (i.e., due to a heterologous insertion within the coding sequences of coding sequences to a ORFX peptide) that are utilized to “knockout” endogenous function of a ORFX peptide by homologous recombination (see, e.g., Capecchi, 1989, Science 244: 1288-1292); or (v) modulators (i.e., inhibitors, agonists and antagonists, including additional peptide mimetic of the invention or antibodies specific to a peptide of the invention) that alter the interaction between a ORFX peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, a ORFX peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of a ORFX peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e g., Northern assays, dot blots, in situ hybridization, etc.).

In one aspect, the invention provides a method for preventing, in a subject, a disease or condition associated with an aberrant ORFX expression or activity, by administering to the subject an agent that modulates ORFX expression or at least one ORFX activity. Subjects at risk for a disease that is caused or contributed to by aberrant ORFX expression or activity can be identified by, for example, any or a combination of diagnostic or prognostic assays as described herein. Administration of a prophylactic agent can occur prior to the manifestation of symptoms characteristic of the ORFX aberrancy, such that a disease or disorder is prevented or, alternatively, delayed in its progression. Depending on the type of ORFX aberrancy, for example, a ORFX agonist or ORFX antagonist agent can be used for treating the subject. The appropriate agent can be determined based on screening assays described herein.

Another aspect of the invention pertains to methods of modulating ORFX expression or activity for therapeutic purposes. The modulatory method of the invention involves contacting a cell with an agent that modulates one or more of the activities of ORFX protein activity associated with the cell. An agent that modulates ORFX protein activity can be an agent as described herein, such as a nucleic acid or a protein, a naturally-occurring cognate ligand of a ORFX protein, a peptide, a ORFX peptidomimetic, or other small molecule. In one embodiment, the agent stimulates one or more ORFX protein activity. Examples of such stimulatory agents include active ORFX protein and a nucleic acid molecule encoding ORFX that has been introduced into the cell. In another embodiment, the agent inhibits one or more ORFX protein activity. Examples of such inhibitory agents include antisense ORFX nucleic acid molecules and anti-ORFX antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a ORFX protein or nucleic acid molecule. In one embodiment, the method involves administering an agent (e.g., an agent identified by a screening assay described herein), or combination of agents that modulates (e.g., upregulates or downregulates) ORFX expression or activity. In another embodiment, the method involves administering a ORFX protein or nucleic acid molecule as therapy to compensate for reduced or aberrant ORFX expression or activity.

Determination of the Biological Effect of a Therapeutic

In various embodiments of the present invention, suitable in vitro or in vivo assays are utilized to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Malignancies

Some ORFX polypeptides are expressed in cancerous cells (see, e.g., Tables 1 and 2). Accordingly, the corresponding ORF protein is involved in the regulation of cell proliferation. Accordingly, Therapeutics of the present invention may be useful in the therapeutic or prophylactic treatment of diseases or disorders that are associated with cell hyperproliferation and/or loss of control of cell proliferation (e.g., cancers, malignancies and tumors). For a review of such hyperproliferation disorders, see e.g., Fishman, et al., 1985. MEDICINE, 2nd ed., J. B. Lippincott Co., Philadelphia, Pa.

Therapeutics of the present invention may be assayed by any method known within the art for efficacy in treating or preventing malignancies and related disorders. Such assays include, but are not limited to, in vitro assays utilizing transformed cells or cells derived from the patient's tumor, as well as in vivo assays using animal models of cancer or malignancies. Potentially effective Therapeutics are those that, for example, inhibit the proliferation of tumor-derived or transformed cells in culture or cause a regression of tumors in animal models, in comparison to the controls.

In the practice of the present invention, once a malignancy or cancer has been shown to be amenable to treatment by modulating (i.e., inhibiting, antagonizing or agonizing) activity, that cancer or malignancy may subsequently be treated or prevented by the administration of a Therapeutic that serves to modulate protein function.

Premalignant conditions

The Therapeutics of the present invention that are effective in the therapeutic or prophylactic treatment of cancer or malignancies may also be administered for the treatment of pre-malignant conditions and/or to prevent the progression of a pre-malignancy to a neoplastic or malignant state. Such prophylactic or therapeutic use is indicated in conditions known or suspected of preceding progression to neoplasia or cancer, in particular, where non-neoplastic cell growth consisting of hyperplasia, metaplasia or, most particularly, dysplasia has occurred. For a review of such abnormal cell growth see e.g., Robbins & Angell, 1976. BASIC PATHOLOGY, 2nd ed., W. B. Saunders Co., Philadelphia, Pa.

Hyperplasia is a form of controlled cell proliferation involving an increase in cell number in a tissue or organ, without significant alteration in its structure or function. For example, it has been demonstrated that endometrial hyperplasia often precedes endometrial cancer. Metaplasia is a form of controlled cell growth in which one type of mature or fully differentiated cell substitutes for another type of mature cell. Metaplasia may occur in epithelial or connective tissue cells. Dysplasia is generally considered a precursor of cancer, and is found mainly in the epithelia. Dysplasia is the most disorderly form of non-neoplastic cell growth, and involves a loss in individual cell uniformity and in the architectural orientation of cells. Dysplasia characteristically occurs where there exists chronic irritation or inflammation, and is often found in the cervix, respiratory passages, oral cavity, and gall bladder.

Alternatively, or in addition to the presence of abnormal cell growth characterized as hyperplasia, metaplasia, or dysplasia, the presence of one or more characteristics of a transformed or malignant phenotype displayed either in vivo or in vitro within a cell sample derived from a patient, is indicative of the desirability of prophylactic/therapeutic administration of a Therapeutic that possesses the ability to modulate activity of An aforementioned protein. Characteristics of a transformed phenotype include, but are not limited to: (i) morphological changes; (ii) looser substratum attachment; (iii) loss of cell-to-cell contact inhibition; (iv) loss of anchorage dependence; (v) protease release; (vi) increased sugar transport; (vii) decreased serum requirement; (viii) expression of fetal antigens, (ix) disappearance of the 250 kDal cell-surface protein, and the like. See e.g., Richards, et al., 1986. MOLECULAR PATHOLOGY, W. B. Saunders Co., Philadelphia, Pa.

In a specific embodiment of the present invention, a patient that exhibits one or more of the following predisposing factors for malignancy is treated by administration of an effective amount of a Therapeutic: (i) a chromosomal translocation associated with a malignancy (e.g., the Philadelphia chromosome (bcr/abl) for chronic myelogenous leukemia and t(14;18) for follicular lymphoma, etc.); (ii) familial polyposis or Gardner's syndrome (possible forerunners of colon cancer); (iii) monoclonal gammopathy of undetermined significance (a possible precursor of multiple myeloma) and (iv) a first degree kinship with persons having a cancer or pre-cancerous disease showing a Mendelian (genetic) inheritance pattern (e.g., familial polyposis of the colon, Gardner's syndrome, hereditary exostosis, polyendocrine adenomatosis, Peutz-Jeghers syndrome, neurofibromatosis of Von Recklinghausen, medullary thyroid carcinoma with amyloid production and pheochromocytoma, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia and Bloom's syndrome).

In another embodiment, a Therapeutic of the present invention is administered to a human patient to prevent the progression to breast, colon, lung, pancreatic, or uterine cancer, or melanoma or sarcoma.

Hyperproliferative and Dysproliferative Disorders

In one embodiment of the present invention, a Therapeutic is administered in the therapeutic or prophylactic treatment of hyperproliferative or benign dysproliferative disorders. The efficacy in treating or preventing hyperproliferative diseases or disorders of a Therapeutic of the present invention may be assayed by any method known within the art. Such assays include in vitro cell proliferation assays, in vitro or in vivo assays using animal models of hyperproliferative diseases or disorders, or the like. Potentially effective Therapeutics may, for example, promote cell proliferation in culture or cause growth or cell proliferation in animal models in comparison to controls.

Specific embodiments of the present invention are directed to the treatment or prevention of cirrhosis of the liver (a condition in which scarring has overtaken normal liver regeneration processes); treatment of keloid (hypertrophic scar) formation causing disfiguring of the skin in which the scarring process interferes with normal renewal; psoriasis (a common skin condition characterized by excessive proliferation of the skin and delay in proper cell fate determination); benign tumors; fibrocystic conditions and tissue hypertrophy (e.g., benign prostatic hypertrophy).

Neurodegenerative Disorders

Some ORFX proteins are found in cell types have been implicated in the deregulation of cellular maturation and apoptosis, which are both characteristic of neurodegenerative disease. Accordingly, Therapeutics of the invention, particularly but not limited to those that modulate (or supply) activity of an aforementioned protein, may be effective in treating or preventing neurodegenerative disease. Therapeutics of the present invention that modulate the activity of an aforementioned protein involved in neurodegenerative disorders can be assayed by any method known in the art for efficacy in treating or preventing such neurodegenerative diseases and disorders. Such assays include in vitro assays for regulated cell maturation or inhibition of apoptosis or in vivo assays using animal models of neurodegenerative diseases or disorders, or any of the assays described below. Potentially effective Therapeutics, for example but not by way of limitation, promote regulated cell maturation and prevent cell apoptosis in culture, or reduce neurodegeneration in animal models in comparison to controls.

Once a neurodegenerative disease or disorder has been shown to be amenable to treatment by modulation activity, that neurodegenerative disease or disorder can be treated or prevented by administration of a Therapeutic that modulates activity. Such diseases include all degenerative disorders involved with aging, especially osteoarthritis and neurodegenerative disorders.

Disorders Related to Organ Transplantation

Some ORFX can be associated with disorders related to organ transplantation, in particular but not limited to organ rejection. Therapeutics of the invention, particularly those that modulate (or supply) activity, may be effective in treating or preventing diseases or disorders related to organ transplantation. Therapeutics of the invention (particularly Therapeutics that modulate the levels or activity of an aforementioned protein) can be assayed by any method known in the art for efficacy in treating or preventing such diseases and disorders related to organ transplantation. Such assays include in vitro assays for using cell culture models as described below, or in vivo assays using animal models of diseases and disorders related to organ transplantation, see e.g., below. Potentially effective Therapeutics, for example but not by way of limitation, reduce immune rejection responses in animal models in comparison to controls.

Accordingly, once diseases and disorders related to organ transplantation are shown to be amenable to treatment by modulation of activity, such diseases or disorders can be treated or prevented by administration of a Therapeutic that modulates activity.

Cardiovascular Disease

GENX has been implicated in cardiovascular disorders, including in atherosclerotic plaque formation. Diseases such as cardiovascular disease, including cerebral thrombosis or hemorrhage, ischemic heart or renal disease, peripheral vascular disease, or thrombosis of other major vessel, and other diseases, including diabetes mellitus, hypertension, hypothyroidism, cholesterol ester storage disease, systemic lupus erythematosus, homocysteinemia, and familial protein or lipid processing diseases, and the like, are either directly or indirectly associated with atherosclerosis. Accordingly, Therapeutics of the invention, particularly those that modulate (or supply) activity or formation may be effective in treating or preventing atherosclerosis-associated diseases or disorders. Therapeutics of the invention (particularly Therapeutics that modulate the levels or activity) can be assayed by any method known in the art, including those described below, for efficacy in treating or preventing such diseases and disorders.

A vast array of animal and cell culture models exist for processes involved in atherosclerosis. A limited and non-exclusive list of animal models includes knockout mice for premature atherosclerosis (Kurabayashi and Yazaki, 1996, Int. Angiol. 15: 187-194), transgenic mouse models of atherosclerosis (Kappel et al., 1994, FASEB J. 8: 583-592), antisense oligonucleotide treatment of animal models (Callow, 1995, Curr. Opin. Cardiol. 10: 569-576), transgenic rabbit models for atherosclerosis (Taylor, 1997, Ann. N.Y. Acad. Sci 811: 146-152), hypercholesterolemic animal models (Rosenfeld, 1996, Diabetes Res. Clin. Pract. 30 Suppl.: 1-11), hyperlipidemic mice (Paigen et al., 1994, Curr. Opin. Lipidol. 5: 258-264), and inhibition of lipoxygenase in animals (Sigal et al., 1994, Ann. N.Y. Acad. Sci. 714: 211-224). In addition, in vitro cell models include but are not limited to monocytes exposed to low density lipoprotein (Frostegard et al., 1996, Atherosclerosis 121: 93-103), cloned vascular smooth muscle cells (Suttles et al., 1995, Exp. Cell Res. 218: 331-338), endothelial cell-derived chemoattractant exposed T cells (Katz et al., 1994, J. Leukoc. Biol. 55: 567-573), cultured human aortic endothelial cells (Farber et al., 1992, Am. J. Physiol. 262: H1088-1085), and foam cell cultures (Libby et al., 1996, Curr Opin Lipidol 7: 330-335). Potentially effective Therapeutics, for example but not by way of limitation, reduce foam cell formation in cell culture models, or reduce atherosclerotic plaque formation in hypercholesterolemic mouse models of atherosclerosis in comparison to controls.

Accordingly, once an atherosclerosis-associated disease or disorder has been shown to be a amenable to treatment by modulation of activity or formation, that disease or disorder can be treated or prevented by administration of a Therapeutic that modulates activity.

Cytokine and Cell Proliferation/Differentiation Activity

A GENX protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+(preB M+), 2E8, RB5, DA1, 123, Ti 165, HT2, CTLL2, TF-1, Mo7e and CMK.

The activity of a protein of the invention may, among other means, be measured by the following methods: Assays for T-cell or thymocyte proliferation include without limitation those described in: CURRENT PROTOCOLS IN IMMUNOLOGY, Ed by Coligan et al., Greene Publishing Associates and Wiley-Interscience (Chapter 3 and Chapter 7); Takai et al., J Immunol 137:3494-3500, 1986; Bertagnoili et al., J Immunol 145:1706-1712, 1990; Bertagnolli etal., Cell Immunol 133:327-341, 1991; Bertagnolli, et al., J Immunol 149:3778-3783, 1992; Bowman et al., J Immunol 152:1756-1761, 1994.

Assays for cytokine production and/or proliferation of spleen cells, lymph node cells or thymocytes include, without limitation, those described by Kruisbeek and Shevach, In: CURRENT PROTOCOLS IN IMMUNOLOGY. Coligan et al., eds. Vol 1, pp. 3.12.1-14, John Wiley and Sons, Toronto 1994; and by Schreiber, In: CURRENT PROTOCOLS IN IMMUNOLOGY. Coligan eds. Vol 1 pp. 6.8.1-8, John Wiley and Sons, Toronto 1994.

Assays for proliferation and differentiation of hematopoietic and lymphopoietic cells include, without limitation, those described by Bottomly et al., In: CURRENT PROTOCOLS IN IMMUNOLOGY. Coligan et al., eds. Vol 1 pp. 6.3.1-6.3.12, John Wiley and Sons, Toronto 1991; deVries et al., J Exp Med 173:1205-1211, 1991; Moreau et al., Nature 336:690-692, 1988; Greenberger et al., Proc Natl Acad Sci U.S.A. 80:2931-2938, 1983; Nordan, In: CURRENT PROTOCOLS IN IMMUNOLOGY. Coligan et al., eds. Vol 1 pp. 6.6.1-5, John Wiley and Sons, Toronto 1991; Smith etal., Proc Natl Acad Sci U.S.A. 83:1857-1861, 1986; Measurement of human Interleukin 11-Bennett, et al. In: CURRENT PROTOCOLS IN IMMUNOLOGY. Coligan et al., eds. Vol 1 pp. 6.15.1 John Wiley and Sons, Toronto 1991; Ciarletta, et al., In: CURRENT PROTOCOLS IN IMMUNOLOGY. Coligan et al., eds. Vol 1 pp. 6.13.1, John Wiley and Sons, Toronto 1991.

Assays for T-cell clone responses to antigens (which will identify, among others, proteins that affect APC-T cell interactions as well as direct T-cell effects by measuring proliferation and cytokine production) include, without limitation, those described In: CURRENT PROTOCOLS IN IMMUNOLOGY. Coligan et al., eds., Greene Publishing Associates and Wiley-Interscience (Chapter 3Chapter 6, Chapter 7); Weinberger et al., Proc Natl Acad Sci USA 77:6091-6095, 1980; Weinberger et al., Eur J Immun 11:405-411, 1981; Takai et al., J Immunol 137:3494-3500, 1986; Takai etal., J Immunol 140:508-512, 1988.

Immune Stimulating or Suppressing Activity

A GENX protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by vital (e.g., HIV) as well as bacterial or fungal infections, or may result from autoimmune disorders. More specifically, infectious diseases causes by vital, bacterial, fungal or other infection may be treatable using a protein of the present invention, including infections by HIV, hepatitis viruses, herpesviruses, mycobacteria, Leishmania species., malaria species. and various fungal infections such as candidiasis. Of course, in this regard, a protein of the present invention may also be useful where a boost to the immune system generally may be desirable, i e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitus, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to immune responses, in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or energy in T cells, is distinguishable from immunosuppression in that it is generally antigen-specific and persists after exposure to the tolerizing agent has ceased. Operationally, tolerance can be demonstrated by the lack of a T cell response upon re-exposure to specific antigen in the absence of the tolerizing agent.

Down regulating or preventing one or more antigen functions (including without limitation B lymphocyte antigen functions (such as, for example, B7), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to energize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4Ig fusion proteins in vivo as described in Lenschow et al., Science 257:789-792 (1992) and Turka et al., Proc Natl Acad Sci USA, 89:11102-11105 (1992). In addition, murine models of GVHD (see Paul ed., FUNDAMENTAL IMMUNOLOGY, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and auto-antibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of auto-antibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythematosis in MRL/lpr/lpr mice or NZB hybrid mice, murine autoimmune collagen arthritis, diabetes mellitus in NOD mice and BB rats, and murine experimental myasthenia gravis (see Paul ed., FUNDAMENTAL IMMUNOLOGY, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response. For example, enhancing an immune response through stimulating B lymphocyte antigen function may be useful in cases of viral infection. In addition, systemic vital diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-vital immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex vivo with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II a chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods: Suitable assays for thymocyte or splenocyte cytotoxicity include, without limitation, those described In: CURRENT PROTOCOLS IN IMMUNOLOGY. Coligan et al., eds. Greene Publishing Associates and Wiley-Interscience (Chapter 3, Chapter 7); Herrmann et al., Proc Natl Acad Sci USA 78:2488-2492, 1981; Herrmann et al., J Immunol 128:1968-1974, 1982; Handa et al., J Immunol 135:1564-1572, 1985; Takai etal., J Immunol 137:3494-3500, 1986; Takai etal., J Immunol 140:508-512, 1988; Herrmann et al., Proc Natl Acad Sci USA 78:2488-2492, 1981; Herrmann et al., J Immunol 128:1968-1974, 1982; Handa et al., J Immunol 135:1564-1572, 1985; Takai et al., J Immunol 137:3494-3500, 1986; Bowman et al., J Virology 61:1992-1998; Takai et al., J lmmunol 140:508-512, 1988; Bertagnolli et al., Cell Immunol 133:327-341, 1991; Brown et al., J Immunol 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J Immunol 144:3028-3033, 1990; and Mond and Brunswick In: CURRENT PROTOCOLS IN IMMUNOLOGY. Coligan et al., (eds.) Vol 1 pp. 3.8.1-3.8.16, John Wiley and Sons, Toronto 1994.

Mixed lymphocyte reaction (MLR) assays (which will identify, among others, proteins that generate predorninantly Th1 and CTL responses) include, without limitation, those described In: CURRENT PROTOCOLS IN IMMUNOLOGY. Coligan et al., eds. Greene Publishing Associates and Wiley-Interscience (Chapter 3, Chapter 7); Takai et al., J Immunol 137:3494-3500, 1986; Takai et al., J Immunol 140:508-512, 1988; Bertagnolli et al, J Immunol 149:3778-3783, 1992.

Dendritic cell-dependent assays (which will identify, among others, proteins expressed by dendritic cells that activate naive T-cells) include, without limitation, those described in: Guery et al., J Immunol 134:536-544, 1995; Inaba et al., J Exp Med 173:549-559, 1991; Macatonia et al., J Immunol 154:5071-5079, 1995; Porgador et al., J Exp Med 182:255-260, 1995; Nair et al, J Virol 67:4062-4069, 1993; Huang et al., Science 264:961-965, 1994; Macatonia et al., J Exp Med 169:1255-1264, 1989; Bhardwaj et al., J Clin Investig 94:797-807, 1994; and Inaba et al., J Exp Med 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Res 53:1945-1951, 1993; Itoh et al., Cell 66:233-243, 1991; Zacharchuk, J Immunol 145:4037-4045, 1990; Zamai et al., Cytometry 14:891-897, 1993; Gorczyca et al., Internat J Oncol 1:639-648, 1992.

Assays for proteins that influence early steps of T-cell commitment and development include, without limitation, those described in: Antica et al, Blood 84:111-117, 1994; Fine et al., Cell Immunol 155: 111-122, 1994; Galy et al., Blood 85:2770-2778, 1995; Toki et al., Proc Nat Acad Sci USA 88:7548-7551, 1991.

Hematopoiesis Regulating Activity

A GENX protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Mol. Cell. Biol. 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, In: CULTURE OF HEMATOPOIETIC CELLS. Freshney, et al. (eds.) Vol pp. 265-268, Wiley-Liss, Inc., New York, N.Y. 1994; Hirayama et al., Proc Natl Acad Sci USA 89:5907-5911, 1992; McNiece and Briddeli, In: CULTURE OF HEMATOPOIETIC CELLS. Freshney, et al. (eds.) Vol pp. 23-39, Wiley-Liss, Inc., New York, N.Y. 1994; Neben et al., Exp Hematol 22:353-359, 1994; Ploemacher, In: CULTURE OF HEMATOPOIETIC CELLS. Freshney, et al. eds. Vol pp. 1-21, Wiley-Liss, Inc., New York, N.Y. 1994; Spoonceret al., In: CULTURE OF HEMATOPOIETIC CELLS. Freshhey, et al., (eds.) Vol pp. 163-179, Wiley-Liss, Inc., New York, N.Y. 1994; Sutherland, In: CULTURE OF HEMATOPOIETIC CELLS. Freshney, et al., (eds.) Vol pp. 139-162, Wiley-Liss, Inc., New York, N.Y. 1994.

Tissue Growth Activity

A GENX protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The -compositions of the invention may also be useful in the treatment of tendonitis, carpal tunnel syndrome and other tendon or ligament defects. The compositions may also include an appropriate matrix and/or sequestering agent as a career as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, EPIDERMAL WOUND HEALING, pp. 71-112 (Maibachi and Rovee, eds.), Year Book Medical Publishers, Inc., Chicago, as modified by Eaglstein and Menz, J Invest. Dermatol 71:382-84 (1978).

Activin/Inhibin Activity

A GENX protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-b group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc Natl Acad Sci USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: CURRENT PROTOCOLS IN IMMUNOLOGY, Coligan et al., eds. (Chapter 6.12, MEASUREMENT OF ALPHA AND BETA CHEMOKINES 6.12.1-6.12.28); Taub et al. J Clin Invest 95:1370-1376, 1995; Lind et al. APMIS 103:140-146, 1995; Muller et al., Eur J Immunol 25: 1744-1748; Gruberet al. J Immunol 152:5860-5867, 1994; Johnston et al., J Immunol 153: 1762-1768, 1994.

Hemostatic and Thrombolytic Activity

A protein of the invention may also exhibit hemostatic or thrombolytic activity. As a result, such a protein is expected to be useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al, J Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: CURRENT PROTOCOLS IN IMMUNOLOGY, Ed by Coligan, et al., Greene Publishing Associates and Wiley-Interscience (Chapter 7.28, Measurement of Cellular Adhesion under static conditions 7.28.1-7.28.22), Takai et al., Proc Natl Acad Sci USA 84:6864-6868, 1987; Bierer et al., J Exp. Med. 168:1145-1156, 1988; Rosenstein et al., J Exp. Med. 169:149-160 1989; Stoltenborg et al., J Immunol Methods 175:59-68, 1994; Stitt et al, Cell 80:661-670, 1995.

Anti-inflammatory Activity

Proteins of the present invention may also exhibit anti-inflammatory activity. The anti-inflammatory activity may be achieved by providing a stimulus to cells involved in the inflammatory response, by inhibiting or promoting cell—cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via ADCC). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Neural disorders in general include Parkinson's disease, Alzheimer's disease, Huntington's disease, multiple sclerosis, amyotrophic lateral sclerosis (ALS), peripheral neuropathy, tumors of the nervous system, exposure to neurotoxins, acute brain injury, peripheral nerve trauma or injury, and other neuropathies, epilepsy, and/or tremors.

TABLE 1

| | | | | |
|---|---|---|---|---|
| TABLE | | | | |
| NO | ACCNO | NUCL. SEQUENCE | Prot SEQUENCE | HOMOLOGY |

| | | | | | |
|---|---|---|---|---|---|
| 1. | 11612531/116 | ncctgattggaggggaattcttcctgctgggtctcat | LIGGEFFLLGLMAYDRYVAV | similar to gi\|3983382 | |
| | 12531 | ggcctatgaccgctatgtggctgtgtgcaaccctct | CNPLRYPLLMNRRVCLFMV | olfactory receptor E3 | |
| | | acggtaccctctcctcatgaaccgcagggtttgctta | VGSWVGGSLDGFMLTPVT | [Mus | |
| | | ttcatggtggtcggctcctgggttggtggttccttggat | MSFPFCRSREINHFFCEIPA | musculus] | |
| | | gggttcatgctgactcctgtcactatgagtttccccttc | VLKLSCTDTSLYETLMYACC | | |
| | | tgtagatcccgagagatcaatcactttttctgtgagat | VL (SEQ ID NO:2) | | |
| | | cccagccgtgctgaagttgtcttgcacagacacgtc | | | |
| | | actctatgagaccctgatgtatgcctgctgcgtgctg | | | |
| | | at (SEQ ID NO:1) | | | |
| 2. | 16401346/164 | aagccgtggttgcccagaggtagaagccgtggttg | CRIISCGELPIPPNGHRIGTL | similar to gi\|1255889 | |
| | 01346 | cccaggaggtagaagccgtggttgcccaggaggt | SVYGATAIFSCNSGYTLVGS | T07H6.5 gene | |
| | | agaagccgtggttgcccaggaggtagaagccgtg | RVRECMANGLWSGSEVRC | product | |
| | | gttgcccaggaggtagaagccgtggtgcccagga | LAGHCGTPEPIVNGHINGE | [Caenorhabitis | |
| | | ggtagaagccgtggttgcaccggtaagacacagt | NYSYRGSVVYQCNAGFRLI | elegans] | |
| | | ggtgccgaagctgaacctgtgcgggccgctggcg | GMSVRICQQDHHWSGKTP | | |
| | | tggacctgacgaacactattttcttggtgtccaggat | FCVPITCGHPGNPVNGLTQ | | |
| | | ctgtacagttgat (SEQ ID NO:3) | GNQFNLNDW (SEQ ID | | |
| | | | NO:4) | | |
| 3. | 17939072/179 | gcaggcaggagttggggtcgtcaagtttgccagcc | RPRDCLDVLLSGQQDDGV | similar to gi\|2506403\| | |
| | 39072 | cccatcagcagaggcaagagatcagtgagctgc | YSVFPTHYPAGFQVYCDMR | MICROFIBRIL- | |
| | | caaatggagggggtggggacggcgagaaggcg | TDGGGWTVFQRREDGSVN | ASSOCIATED | |
| | | atgtgtgacttcaccggcccagggagcttcgtgtca | FFRGWDAYRDGFGRLTGE | GLYCOPROTEIN | |
| | | gggatggcccggcccctccctactggagagtggg | HWLGLKRIHALTTQAAYELH | 4 | |
| | | gccaggcacaggtgggtggagaacattcacg | VDLEDFENGTAYARYGSFG | | |
| | | aaagagtgaggtggggtcggggatggggcgaca | VGLFSVDPEEDGYPLTVAD | | |
| | | gggaccagcagggccaag (SEQ ID NO:5) | YSGTAGDSLLKHS (SEQ ID | | |
| | | | NO:6) | | |
| 4. | 11709218/117 | cggtgatgaaggcaggctccgtgctgggtgggaa | KGPSCDRCKPGFWGLSPS | similar to gi\|2851576\| | |
| | 09218 | gggccacgggctgactctgtgctgtgcagttggcac | NPEGCTRCSCDLRGTLGG | LAMININ ALPHA-5 | |
| | | aggcggccgacctgccctcctctcgcacagagac | VAECQAGTGQCFCKPHVC | CHAIN | |
| | | ccgcccgctcacactcatggccccccggttgacgt | GQACASCKDGFFGLDQAD | | |
| | | atcggaagacgagccagaaaaggtcgggggag | YFGCRSCRCDIGGALGQSC | | |
| | | gtcaggttcagcctggccacgatcctgggctggac | EPRTGVCRCRPNTQGPTC | | |
| | | aggtgccatctgcgcgtagcccctccagctgaagtt | SEPARDHYLPDLHHLRLEL | | |
| | | ctcgaactcgaggg (SEQ ID NO:7) | EEAATPEGHAVRFGFN | | |
| | | | (SEQ ID NO:8) | | |
| 5. | 17893306/178 | aacctgcgggagctttggctctatgacaaccacat | NLRELWLYDNHISSLPDNVF | similar to gi\|3183012\| | |
| | 93306 | ctcttctctacccgacaatgtcttcagcaacctccgc | SNLRQLQVLILSRNQISFISP | PLATELET | |
| | | cagttgcaggtcctgattcttagccgcaatcagatc | G (SEQ ID NO:10) | GLYCOPROTEIN V | |
| | | agcttcatctccccgggtgccttcaacgg (SEQ | | PRECURSOR | |
| | | ID NO:9) | | | (GPV) (CD42D) |
| 6. | 16305313/163 | accggggctgtgccacccagccatgccaccacg | CATQPCHHGALCVPQGSR | similar to gi\|499686 | |
| | 05313 | gcgctctgtgtgtgccccagggtccagatcccaac | PNGFRCYCVPGFQGPRCE | fibropellin Ia | |
| | | ggcttccgctgctactgcgtgccgggtttccagggc | LDIDECASRPCHHGATCRN | [Heliocidaris | |
| | | ccacgctgcgagctggacatcgatgagtgtgcatc | LADRYECHCPLGYAGVTCE | erythrogramma] | |
| | | ccggccgtgccaccatggggccacctgccgcaa | TEVDECASAPC(SEQ ID | | |
| | | cctggccgatcgctacgagtgccattgcccccttgg | NO:12) | | |
| | | ctatgcaggcgtgacctgcgagacggaggtggac | | | |
| | | gagtgcgcctcagcgccctgcctgcacg (SEQ | | | |
| | | ID NO:11) | | | |
| 7. | 21652877/216 | acatacaggtaccactcaaatgaatgagcactcc | HTGTTQMNEHSSRSHAIFTI | similar to gi\|1703701\| | |
| | 52877 | agcagatcacatgcaatttttacaatcagcatttgtc | SICQVHKNMEAAEDGSWY | KRP5=kinesin- | |
| | | aagttcataaaaatatggaggcagctgaagatgg | SPRHIVSKFHFVDLAG(SEQ | related | |
| | | atcatggtattcccctcggcatattgtctcaaagttcc | ID NO:14) | protein [rats, | |
| | | actttgtggatttggcaggatcagaaagagtaacc | | testes, Peptide | |
| | | aaaacggggaatactggtgaacggttcaaagaat | | Partial, 167 aa] | |
| | | ccattcaaatcaatagtggattgctggctttaggaa | | | |
| | | atgtaataagcgctcttg (SEQ ID NO:13) | | | |
| 8. | 16305313/163 | accggggctgtgccacccagccatgccaccacg | CATQPCHHGALCVPQGSR | similar to gi\|499686 | |
| | 05313 | gcgctctgtgtgtgccccagggtccagatcccaac | PNGFRCYCVPGFQGPRCE | fibropellin Ia | |
| | | ggcttccgctgctactgcgtgccgggtttccagggc | LDIDECASRPCHHGATCRN | [Heliocidaris | |
| | | ccacgctgcgagctggacatcgatgagtgtgcatc | LADRYECHCPLGYAGVTCE | erythrogramma] | |
| | | ccggccgtgccaccatggggccacctgccgcaa | TEVDECASAPC(SEQ ID | | |
| | | cctggccgatcgctacgagtgccattgcccccttgg | NO:16) | | |
| | | ctatgcaggcgtgacctgcgagacggaggtggac | | | |
| | | gagtgcgcctcagcgccctgcctgcacg (SEQ | | | |
| | | ID NO:15) | | | |
| 9. | 16296826/162 | gggcccttccgaggctgcctccaggacctgcgact | DMCSPDPCFNGGTCLVTW | similar to gi\|2373395\| | |
| | 96826 | cgatggctgccacctccccttctttcctctgccactgg | NDFHCTCPANFTGPTCAQQ | cell surface protein | |
| | | ataactcaagccagcccagcgagctcggcggca | LWCPGQPCLPPATCV(SEQ | [Mus | |
| | | ggcagtcctggaacctctctttggggccttgcgtctc | ID NO:18) | musculus] | |
| | | cgaggacatgtgcagtcctgacccctgtttcaatgg | | | |
| | | tgggacttgcctcgtcacctggaatgacttccactgt | | | |
| | | acctgccctgccaatttcacggggcctacatgtgcc | | | |
| | | cagcagctgtggtgtcccggccagccctgtctccc | | | |
| | | acctgccacgtgtgtggcggaggccacgttccgcg | | | |
| | | agggtccccccgccgcgttcagcgggcacaacg | | | |
| | | cgt (SEQ ID NO:17) | | | |
| 10. | 27981100/279 | tcatgattgggggcccaacaggaggctgaggaat | HDWGPNRRLRNGFSQIHC | similar to gi\|1657758 | |
| | 81100 | ggcttttctcaaatccactgccgcccggaatcttcta | RPESSKGVISISKSTERLSP | densin-180 [Rattus | |
| | | aaggtgttatttcaattagcaaaagcacagagagg | LMKDIKSNKFKKSQSIDEIDI | norvegicus] | |
| | | ctttcccccctaatgaaagatatcaagtctaataaat | GTYKVYNIPLENYASGSDHL | | |
| | | tcaaaaagtcacagagtatcgatgagattgacatt | GSHERPDKMLGPEHGMSS | | |
| | | ggtacatataaggtgtataacataccattagaaaa | MSRNQSVPMLGRE(SEQ ID | | |
| | | ctatgcttctgggagtgatcacttaggaagccacga | NO:20) | | |
| | | acgaccggataagatgctgggaccagagcatggt | | | |
| | | atgtccagtatgtctcgaaaccagtcagtcccaatg | | | |
| | | ctggggcgggaga (SEQ ID NO:19) | | | |
| 11. | 21648376/216 | gtcgacatgacgatcgcggagatgccgggcgcgt | KDKLDAEVHAGEGTPGDVI | similar to | |
| | 48376 | cggttgtcacccgctcgaccgggacgccgagacg | VLRFSGAMAKRPASVILPLL | gi\|3334791\| | |
| | | caatcctagccaggctgccagcagcatcgccggc | LSDSPVIAWWPFSGPDNLA | hypothetical protein | |
| | | gcattaccggcggctgactctacccgagcgaactt | SDPIGALADRRITDSAADKD | SC5A7.10c | |
| | | gacggtcgctggatgttgatccaaagctgctgcag | PCKALIRRAAHLTEGDSDLC | [Streptomyces | |
| | | ctagggctctccagctggtggtgcgagcccaacac | WARTTSWRALAAAALDQH | coelicolor] | |
| | | aggtcggagtcaccctcggttaggtgagccgcac | PATVKFARVESAAGNAPAM | | |
| | | ggcgtatgagggctttgcacggatctttgtcagctgc | LLAAWLGLR(SEQ ID | | |
| | | cgagtcggtgatgcggcggtccgcaagggctccg | NO:22) | | |
| | | atggggtccgaggcgaggttgtcagggccggag | | | |
| | | aagggccaccacgcaatgacgggggagtccga | | | |
| | | cagtagcagcggaaggataactgaggcaggac | | | |
| | | gcttcgccatggctccggaaaaccgcagcacgat | | | |
| | | gacatccccgggggtgccttcaccggcatgcacct | | | |
| | | cggcgtcaagcttgtccttacgagaacgcgt | | | |
| | | (SEQ ID NO:21) | | | |
| 12. | 10286716/102 | acgcgtgcaggcttcaggttggtgacggtgactgtg | IRYYEKGQSEQTYSMVKTG | similar to gi\|2462302\| | |
| | 86716 | ggcgcccctgtcttcaccatggagtaagtctgctca | APTVTVTNLKP(SEQ ID | Eph-like receptor | |
| | | ctctgacccttctcgtagtatcggatcc (SEQ ID | NO:24) | tyrosine | |
| | | NO:23) | kinase [Gallus | | |
| | | | | gallus] | |
| 13. | 29200253/292 | gccggctgtgcgtggatgtgagattggaaccatgtt | RLCVDVRLEPCFLRWDEDE | similar to gi\|627438\| | |
| | 00253 | tcctgcgatgggatgaggatgagtgtggggtcacc | CGVTLPGKYRMDVCCCSIG | fibrillin-2 precursor- | |
| | | ctgcctggcaagtaccggatggacgtctgctgctg | AVWGVECEACPDPESLEFA | human | |
| | | ctccatcggggccgtgtggggagtcgagtgcgag | SLCPRGLGFASRDFLSGRP | | |
| | | gcctgcccggatcccgagtctctggagttcgccag | FYKDVNECKVFPGLCTHGT | | |
| | | cctgtgcccgcgggggctgggcttcgccagccgg | CRNTVGSFHCACAGGFALD | | |
| | | gacttcctgtctggccgaccattctataaagatgtga | AQERNCTDIDECRISPDLCG | | |
| | | atgaatgcaaggtgttccctggcctctgcacgcac | QGTCVNTPGS(SEQ ID | | |
| | | ggtacctgcagaaacacggtgggcagcttccact | NO:26) | | |
| | | gcgcctgtgcggggggcttcgccctggatgcccag | | | |
| | | gaacggaactgcacagatatcgacgagtgtcgca | | | |
| | | tctctcctgacctctgcggccagggcacctgtgtca | | | |
| | | acacgccgggcagctttgagtgcgagtgttttcccg | | | |
| | | gctacgagagtggcttcatgctgatgaagaactgc | | | |
| | | ag (SEQ ID NO:25) | | | |
| 14. | 3885620/3885 | tggtgtcgggttctcntggcgtcgtctcgtctttntttttc | HQRTKKPPREDAPNPKNRP | similar to gi\|1519394 | |
| | 620 | cgtggtgtttcagcgcncctgtcttctggaattgtttcg | WAQGNFKPETPEHPSIKHT | leptin receptor [Homo | |
| | | ggctccagggggggggggccacgtgtcactggtg | APVTRGPPPLEPETIPEDRA | sapiens] | |
| | | ctgtatgcttgatggaggggtgctcaggcgtttctgg | ETPRKKDETTP(SEQ ID | | |
| | | cttctnaaaattnggtccttgtgcccagggacggttc | NO:28) | | |
| | | ttggggttcggggcatcttcccggggtggctttttcgtt | | | |
| | | ctttggtgtggggggtccactagttctggggtctcttttt | | | |
| | | ttaagtttggtggggtatcttccccgtccgcncggtg | | | |
| | | gttgtatncatcatccattgtagtggtattaaanaaa | | | |
| | | aaaaaaaaaaaaaaaaaaaaaaaaaaaaaanaanaaa | | | |
| | | nnnnnnnnnnnnnnnnnncnnngttttttttnntn | | | |
| | | aattaattttttatttaaaaaacaacctttttttnn | | | |
| | | (SEQ ID NO:27) | | | |
| 15. | 19540620/195 | agtacagcattatccagtacttcagtgttactctgctg | YSIIQYFSVTLLYSILSNLGD | similar to gi\|3451312\| | |
| | 40620 | tattctatcttaagtaacctaggagacttccagtttctc | FQFLFIDLAIILVVVFTMSLN | membrane atpase | |
| | | ttcattgatctggcaatcattttggtagtggtatttaca | PAWKELVAQRPPSGLISGA | | |
| | | atgagtttaaatcctgcctggaaagaacttgtggca | LLFSVLSQIII(SEQ ID NO:30) | [Schizosaccharomyces | |
| | | caaagaccaccttcgggcttatatctggggcccttc | | pombe] | |
| | | tcttctccgttttgtctcagattatcatctgcattggattt | | | |
| | | caatctttgggtttt (SEQ ID NO:29) | | | |
| 16. | 3223926/acc:3 | nagctnnatgggcttcagcaacttctgtattacactt | YSFIAKILWRSAKKDVIDQQI | similar to gi\|4895216 | |
| | 223926 | gctcatgtagtgctctcgcagctgtttggcctcttcctc | PPQTEEIHWLHFSPVERHF | putative | |
| | | aagtcggccatcacgcaaggtaggtggtatccctg | YHRQHEVCCQDVVVKLRKI | serine/threonine | |
| | | ggtgcctggctatcaacaattccatcaagttatgggt | SDWALKLSSLDRRTVTSILY | protein kinase | |
| | | agcatgaagtctttgaagtgaatcagttttgagttttc | PLLRLRQACCHPQAVRGEF | [Arabidopsis thaliana] | |
| | | ctttgtgttcctccgaggagcgcaacacttctctgtac | LPLQKSTMTMEELLTSLQK | | |
| | | aattctgctgccaaggcatactcacctttaataatat | KCGTECEEAHRQLVCALNG | | |
| | | gaatgcctgctaagccattgagagcacaaactag | LAGIHIIK(SEQ ID NO:32) | | |
| | | ctgtcgatgtgcttcttcacattcagttccacatttcttct | | | |
| | | gcaaagatgtcagcagctcttccattgtcatggtgct | | | |
| | | ttttggagtggcaagaactctccacgaacagcctg | | | |
| | | tgggtgacagcaggcctgtctgagcctcagcaatg | | | |
| | | gatacaggatagaggtgacagtccttctgtctaggc | | | |
| | | tgctgagcttcagagcccagtcagaaatcttcctga | | | |
| | | gttttaccaccacatcctggcagcacacctcatgct | | | |
| | | gacggtgatagaaatgcctttccactggagaaaa | | | |
| | | gtggagccagtgtatttcttcggtttgtggtggtatttg | | | |
| | | gnattggtcaatcacatctttctttgcagacctccac | | | |
| | | agtatcttggcaataaagctgta (SEQ ID | | | |
| | | NO:31) | | | |
| 17. | 55777901/557 | cctagcacgctgaactggtactgggtgtggggctg | CSAQGDPPPVVSWTKVGR | similar to gi\|103105 | |
| | 77901 | cagccctggcactaggaggtgagcagcccccac | GLQGQAQVDSNSSLILRPL | cell adhesion protein | |
| | | aggcactgccaaggacacccagtcatggtgcatt | TKEAHGHWECSASNAVAR | Gp160-Dtrk—fruit | |
| | | cggtcaggacgcttggccagtggggtgtaccaga | VATSTNVYVLGTSPHVVTN | fly (Drosophila | |
| | | cactgaatctctgcagataaccaccatcaaagcca | VSVVALPKGANVSWE(SEQ | melanogaster) | |
| | | ggctcccaggagacattggcacccttgggcaaag | ID NO:34) | | |
| | | ccaccacggacacattggtgacaacatgagggct | | | |
| | | agtgcccagcacgtagacgttcgtggaggtggcc | | | |
| | | actcgggccacagcattgctggcactgcattccca | | | |
| | | gtgcccgtgggcctccttggtcaatggtcgcaggat | | | |
| | | gaggctgctgttgctgtccacctgggcctggccttgc | | | |
| | | agcccccggcccaccttggtccaagagacaaca | | | |
| | | ggaggagggtccccttgggcggagcagggga | | | |
| | | (SEQ ID NO:33) | | | |
| 18. | 15025413/150 | gcgatcaatgaccccactaataaaaggtatgaggt | INDPTNKRYEVPVPLNTPPQ | similar to gi\|90144 | |
| | 25413 | tccagtaccactgaacacccctccccaaccagttg | PVGDPENRLYDVRIQNNPF | sucrose alpha- | |
| | | gtgaccctgaaaaccgtctgtatgatgtcaggattc | GIQIQRKNSSTVIWDSQLPG | glucosidase (EC | |
| | | agaacaatccttttggaatccagattcaacgcaaa | FIFNDMFLSIST(SEQ ID | 3.2.1.48)/oligo- | |
| | | aactccagcactgtgatttgggattctcaactccctg | NO:36) | 1,6-glucosidase (EC | |
| | | gcttcatcttcaatgacatgtttctctccatttctacgc | | 3.2.1.10)- | |
| | | (SEQ ID NO:35) | | rabbit | |
| 19. | 29694725/296 | ggccggggctgcatatctcaaagtgtccccaggg | CPQGHFGPGCEQLCQCQH | similar to gi\|3449294 | |
| | 94725 | ccactttgggcccggctgtgagcagctgtgccagt | GAACDHVSGACTCPAG(SE | MEGF6 [Rattus | |
| | | gtcagcatggagcagcctgtgaccacgtcagcgg | Q ID NO:38) | norvegicus] | |
| | | ggcctgcacctgcccggccggt (SEQ ID | | | |
| | | NO:37) | | | |
| 20. | <221> | gtgcaccaagccggccatcatctttgtcagcaaga | CTKPAIIFVSKKGTLVCANP | similar to gi\|1709026 | |
| | misc_feature | aggggaccttggtctgtgccaaccccagtgatctg | SDLRVQKCIKRLEQNSQPQ | MACROPHAGE | |
| | <222> (0) . . . (0) | agagttcagaagtgcattaaaagattggagcaaa | (SEQ ID NO:40) | INFLAMMATORY | |
| | <223> Internal | actcacaaccacagatct (SEQ ID NO:39) | | PROTEIN 1- | |
| | ID: FLC:2378 | | GAMMA | | |
| | | PRECURSOR (MIP- | | | |
| | <221> | | 1-GAMMA) | | |
| | misc_feature | | (MACROPHAGE | | |
| | <222> (0) . . . (0) | | INFLAMMATORY | | |
| | | | PROTEIN-RELATED | | |
| | | | PROTEIN-2) | | |
| | | | (MRP-2) (CCF18 | | |
| 21. | 35056380/350 | tgccagcaacccctgctggggtgatttgctgtgcatt | CVPPGDCASHPCQNGGSC | similar to gi\|3449286 | |
| | 56380 | aatcagtggtatgcctacaggtgtgtccctcctggg | EPGLHSGFTCSCPDSHTGR | MEGF1 [Rattus | |
| | | gactgtgcctcccacccgtgccagaatggtggcag | TCEM(SEQ ID NO:42) | norvegicus] | |
| | | ctgtgagccaggcctgcactccggcttcacctgtag | | | |
| | | ctgcccagactcgcacacgggaaggacctgtga | | | |
| | | gatggtggtggcctgtcttggcgtcctctgtcctcag | | | |
| | | gga (SEQ ID NO:41) | | | |
| 22. | 38905521/389 | ccctggaggagccgatggtggacctggacggcg | LEEPMVDLDGELPFVRPLP | similar to gi\|3449294 | |
| | 05521 | agctgcctttcgtgcggcccctgccccacattgccg | HIAVLQDELPQLFQDDDVG | MEGF6 [Rattus | |
| | | tgctccaggacgagctgccgcaactcttccaggat | ADEEEAELRGEHTLTEKFV | norvegicus] | |
| | | gacgacgtcggggccgatgaggaagaggcaga | CLDDSFGHDCSLTCDDCRN | | |
| | | gttgcggggcgaacacacgctcacagagaagttt | GGTCLLGLDGCDCPEGWT | | |
| | | gtctgcctggatgactcctttggccatgactgcagct | GVICNEICPP(SEQ ID | | |
| | | tgacctgtgatgactgcaggaacggagggacctg | NO:44) | | |
| | | cctcctgggcctggatggctgtgattgccccgagg | | | |
| | | ggtggactggggttatttgcaatgagatttgtcctcc | | | |
| | | g (SEQ ID NO:43) | | | |
| 23. | 5721829/5721 | ccggatcagggcgggcatctcacgcaggaagtg | PQLCRGLVSVTWGCGVAN | similar to gi\|3983360 | |
| | 829 | gtccacctcgtggtgcccacagcggggtaagcgc | SLAMSPVTLRLPRCGHHEV | olfactory receptor A3 | |
| | | agggtcacaggagacatggccaaggagttggcc | DHFLREMPALIR(SEQ ID | [Mus musculus] | |
| | | accccacagccccaggtcactgacaccaagccc | NO:46) | | |
| | | cggcagagctggggttcatga (SEQ ID | | | |
| | | NO:45) | | | |
| 24. | 29447258/294 | agtacagccttgatcagagctgtcctgttttcactgtc | PGYRVRQKDLGMIHKAAIA | similar to gi\|5689485 | |
| | 47258 | agtgaggttaagctggcattttctggccaccaggtc | GDVNKVMESILLRLNDLND | KIAA1074 protein | |
| | | agctaccactcctggacggccatgggcacacgcc | RDKKNRTALLLACAHGRPG | [Homo | |
| | | aaaagtagagcagtcctgttcttcttgtccctgtcgtt | VVADLVARKCQLNLTDSEN | sapiens] | |
| | | caagtcattcagcctgagcaagatgctctccatcac | RTALIKAV(SEQ ID NO:48) | | |
| | | cttgttcacatcacctgcgatggcagctttgtggatc | | | |
| | | atgcccagatccttctgccggactcggtacccggg | | | |
| | | (SEQ ID NO:47) | | | |
| 25. | 10131798/101 | aggccaagcagcaaaaaccacagatcattgctat | AKQQKPQIIAMGNVSFSCS | similar to gi\|1857710\| | |
| | 31798 | gggaaatgtgtcattttcttgttcacaaccacaatcta | QPQSMPVTFLSSRSFLALP | contactin associated | |
| | | tgcccgtgacttttctgagctccaggagttttttagca | DFSGEEEVSATFQFRTWNK | protein [Rattus | |
| | | ctgccagacttctctggagaggaggaggtttctgcc | AGLLLFSELQLISGGILLFLS | norvegicus] | |
| | | acttttcaatttcgaacttggaataaggcagggcttct | DGKLKSNLYQ(SEQ ID | | |
| | | gctgttcagtgaacttcagctgatttcagggggtatc | NO:50) | | |
| | | ctcctctttctgagtgatggaaaacttaagtcgaatct | | | |
| | | ctaccagcca (SEQ ID NO:49) | | | |
| 26. | 150284981/15 | tggaaatacctcttgtgtggggggtgtggaaggga | RFECRCPPGFGGPRCRLP | similar to gi\|3108187 | |
| | 028498 | cctgaggctcctctcactgccccaccccatgacag | VPIKECSLNVTCLDGSPCE | Notch 3 [Homo | |
| | | agtggcccaggaacagccccatggtgctgcgctct | GGSPAANCSCLEGLAGQR | sapiens] | |
| | | ctccccagcagcatctcgccaagggcccagcag | CQVPTLPCEANPCLNGGTC | | |
| | | gccacgtccacggcccctcacccctgacaacact | RAAGGVSECICNARFSGQF | | |
| | | accgccacttcacagaactggccggagaatctg | CEVaV(SEQ ID NO:52) | | |
| | | gcattgcagatacattcagacacccctccagctgc | | | |
| | | ccggcaggtgcccccattcaagcaggggttggctt | | | |
| | | cacaggggagagtggggacctgacacctctggc | | | |
| | | cagcaagaccctccaggcagctgcagttggcagc | | | |
| | | gggagagccaccctcacatgggctgccatcgag | | | |
| | | gcaggtgacattcaggctgcactccttggatggga | | | |
| | | caggcaacctgcagcgcgggcccccgaagcca | | | |
| | | ggcgggcagcggcactcgaagcg (SEQ ID | | | |
| | | NO:51) | | | |
| 27. | 30676688/306 | tgcatatgaaaattgctgcatgtataatatgtccaatt | CLFTHNIYLQDVQMVHPKF | similar to glioma | |
| | 76688 | tttgaacatggagtaacagcacaaccaactttgtaa | YGIGENMWVGPENEFTASI | pathogenesis-related | |
| | | gaatggtcccaaacaagctgaatataattagaac | AIRSWHAEKKMYNFENGSC | protein | |
| | | agtctccagagcaactgccattttcaaaattgtacat | SGDCSNYIQLVWDHSYKVG | -human | |
| | | tttcttctctgcatgccaacttctgatagcaatacttgc | CAVTPCSKI(SEQ ID NO:54) | | |
| | | agtaaattcattttcagggccgacccacatattttca | | | |
| | | ccaataccataaaatttaggatggaccatttgtacat | | | |
| | | cttgtaaataaatattatgcgtaaacaaaca | | | |
| | | (SEQ ID NO:53) | | | |
| 28. | 2911016/2911 | cgattcacatgcccagggcccccgtggcgaagga | RTLMLSVFYFAIPLGSGLGY | similar to gi\|3874275 | |
| | 016 | gacagccgacgtggccagggaggagaagacgt | ITGSSVKQAAGDWHWALR | Yeast low-afinity | |
| | | agctgcggtttcgaatcagggccttcatatctcgga | VSPVLGMITGTLILILVPATK | glucose | |
| | | gccatgaggtccgggccttgagctggtccccgagc | RGHADQLGDQLKARTSWL | transporter HXT4 | |
| | | tggtcggcatgaccccttttagtggctgggaccaga | RDMKALIRNRSYVFSSLATS | (PS:32467);cDNA | |
| | | atgaggatgagtgttcctgtgatcatgcccaggaca | AVSFATGAL(SEQ ID NO:56) | EST | |
| | | ggggacacccgcaatgcccagtgccagtctccgg | | EMBL:C12555 | |
| | | ctgcctgcttcacgctggagccagtaatgtagccca | | com | |
| | | ggccactgcccagtgggatggcgaagtagaaga | | | |
| | | cggacagcatgagcgtacgcgt (SEQ ID | | | |
| | | NO:55) | | | |
| 29. | 16530243/165 | acgcgtcctgcctcgagggcctcgggagcttccgc | ASCLEGLGSFRCLCWPGYS | similar to gi\|117422\| | |
| | 30243 | tgcctctgttggccaggctacagcggcgagctgtg | GELCEVDEDECASSPCQH | CRUMBS PROTEIN | |
| | | cgaggtggacgaggacgagtgtgcatcgagccc | GGRCLQRSDPALYGGVQA | PRECURSOR | |
| | | ctgccagcatgggggccgatgcctgcagcgctctg | AFPGAFSFRHAAGFLCHCP | (95F) | |
| | | acccggccctctacgggggtgtccaggccgccttc | PGFEGADCGVEVDECASR | | |
| | | cctggcgccttcagcttccgccatgctgcgggtttcc | PCLNGGHCQDLPNGFQCH | | |
| | | tgtgccactgccctcctggctttgagggagccgact | CPDGYAGPTCEEDVDECLS | | |
| | | gcggtgtggaggtggacgagtgtgcctcacggcc | DPCLHGGTCSDTVAG(SEQ | | |
| | | atgcctcaacggaggccactgccaggacctgccc | ID NO:58) | | |
| | | aatggcttccagtgtcactgcccagatggctacgc | | | |
| | | agggccgacatgtgaggaagatgtggatgaatgc | | | |
| | | ctgtcggatccctgcctgcacggcggaacctgcag | | | |
| | | tgacactgtggcaggctatatctgcaggtgcccag | | | |
| | | agacctggggtgggcgcgactgttctgtgcagctc | | | |
| | | actggctgccagggccacacctgcccgctggctg | | | |
| | | ccacctgcatccctatcttcgagtctggggtccaca | | | |
| | | gttacgtctgccactgcccacctggtacccatggac | | | |
| | | gttctgtggccagaataccaccttctctgtgatggc | | | |
| | | tgggagccccattcaggcatcagtgccagctggtg | | | |
| | | gccccctgggtctggcactgaggtttcgcaccaca | | | |
| | | ctgcccgctgggaccttggccactcgcaatgacac | | | |
| | | caaggaaagcttggagctggcattggtggcagcc | | | |
| | | acacttcaggccacactctggagctacagcacca | | | |
| | | ctgtgcttgtcctgagactgccggacctggccc | | | |
| | | (SEQ ID NO:57) | | | |
| 30. | 21646822/216 | taccaggtgggcagtggcagacgtaactgtggac | GFLCHCPPGFEGADCGVE | similar to fibropellin | |
| | 46822 | cccagactcgaagatagggatgcaggtggcagc | VDECASRPCLNGGHCQDL | Ia [Heliocidaris | |
| | | cagcgggcaggtgtggccctggcagccagtgag | PNGFQCHCPDGYAGPTCE | erythrogramma] | |
| | | ctgcacagaacagtcgcgcccaccccaggtctct | EDVDECLSDPCLHGGTCSD | | |
| | | gggcacctgcagatatagcctgccacagtgtcact | TVAGYICRCPETWGGRDCS | | |
| | | gcaggttccgccgtgcaggcagggatccgacag | VQLTGCQGHTCPLAATCI(S | | |
| | | gcattcatccacatcttcctcacatgtcggccctgcg | SEQ ID NO:60) | | |
| | | tagccatctgggcagtgacactggaagccattggg | | | |
| | | caggtcctggcagtggcctccgttgaggcatggcc | | | |
| | | gtgaggcacactcgtccacctccacaccgcagtc | | | |
| | | ggctccctcaaagccaggagggcagtggcacag | | | |
| | | gaaacccgcagcatggcggaagctgaaggcgc | | | |
| | | cagggaaggcggcctggacacccccgtagagg | | | |
| | | gccgggtcagagcgctgcaggcatcggcccccat | | | |
| | | gctggcaggggctcgatgcacactcgtcctcgtcc | | | |
| | | acctcgcacagctcgccgctgtagcctggccaac | | | |
| | | agaggcagcggaagctcccgaggccctcgagg | | | |
| | | caggacgcgt (SEQ ID NO:59) | | | |
| 31. | 20720154/207 | tgcacgtgtgcatatgtgtgcgtgtgcgcactcagg | LSSVAACDTGHWGPDCSH | similar to gi\|3449294\| | |
| | 20154 | agggtggcagccaaaggcagggccgggactca | PCNYSAGHGSCDAISGLCL | MEGF6 [Rattus | |
| | | cgctgctcgcaccgcgggcccacgtagccagcct | CEAGYVGPRCEQ(SEQ ID | norvegicus] | |
| | | cacacagacacaggccgctgatggcatcacagct | NO:62) | | |
| | | cccgtggccagcgctgtagttgcaggggtggctgc | | | |
| | | agtcaggtccccagtgcccagtatcacaggctgca | | | |
| | | acagaactcagggtcacccggcgcag (SEQ | | | |
| | | ID NO:61) | | | |
| 32. | 19506719/195 | acatgacagggaaatagtgtggatattttaaggtaa | EQVQNEIKSMKKRMSELCI | similar to gi\|1783127\| | |
| | 06719 | ttttatacttgtcatcatctgtcttttctaaactgtcaatg | DFNKNLNEDDTFLVFSKAEL | endopeptidase 24.16 | |
| | | aaatcatcaggaagagcaccaagttcagccttgg | GALPDDFIDSLEKTDDDKYK | type | |
| | | aaaatacaaggaaggtatcatcctcattgaggttttt | ITLKYPHYFPVM(SEQ ID | M2 [Sus scrofa] | |
| | | gttaaaatcaatacatagctcactcattcttttcttcatt | NO:64) | | |
| | | gatttgatttcattctgtacttgttcagaagatg | | | |
| | | (SEQ ID NO:63) | | | |
| 33. | 16314987/163 | cgcgtgccatgtgcatgtgtatatgcatgtatgtgcgt | CACVYACMCVCVCMCVCIC | similar to gi\|854065 | |
| | 14987 | atgtgtgtgcatgtgtacgtgtgtatatgcatgtgtgta | MCVCACTCVCICVCMHACA | U88 [Human | |
| | | tgtgcatgtacgtgtgtgcatatgcgtgtgcatgcat | YVCIMCTHVHTCTCSCMCT | herpesvirus 6] | |
| | | gcgtgtgcgtatgtgtgcatacatgtgcacacatgta | CICVHVYACTCMSICTRVDV | | |
| | | cacacgtgtacatgttcatgcatgtgcgcacgtgca | CVCMCVC(SEQ ID NO:66) | | |
| | | tatgtgtacacgtgtatgcgtgtacatgtatgagcat | | | |
| | | atgtacacgtgtggatgtgtgtgtatgcatgtgtgtgt | | | |
| | | gcacagatatgccttttcctttcatacaggctgttttga | | | |
| | | gtattgctgttaggcagtgacaactttccgtttcctca | | | |
| | | gtcagaaaatgggtagctcatcattaataaagat | | | |
| | | (SEQ ID NO:65) | | | |
| 34. | 11116197/111 | gtgcactggcccaggtagcgctccacaccagcgc | TARAGDWPELQLVGGSGR | similar to gi\|1480359\| | |
| | 16197 | actggaggttgtccaggaagatgtctcctgagccg | CSGRVEILHQGAWGTVCD | scavenger-receptor | |
| | | gggccaaagtgggcctttccaagggcagacatgg | DLWDLNEAEVVCRQLGCG | protein | |
| | | ctcgaccacacccaagctgccggcacacaacctc | RAMSALGKAHFGPGSGDIF | [Sus scrofa] | |
| | | agcttcgttcaggtcccacaggtcatcacacacggt | LDNLQCAGVERYLGQC(SEQ | | |
| | | gccccaggcgccctggtggagaatctccacgcgt | ID NO:68) | | |
| | | cctgagcaccggccagagccacccaccagctgc | | | |
| | | agctccggccagtcccctgctcttgctgttactgtcct | | | |
| | | gggtgttgctgtcgggatggtgattagatgtggggtc | | | |
| | | tgcaacactgcctctftgggtgcagagtaataataa | | | |
| | | gcaatgaagttttttcctaggttgttgaagcttcggaa | | | |
| | | atacacgagggtcattgagctggaggaggagatg | | | |
| | | taggtgtgttggaagccagagcaggtcctccccaa | | | |
| | | tgactttactgaggatggtggaccgtccaggatttca | | | |
| | | aaatattcattggtgcagtcaaggtcaagatgtgga | | | |
| | | aatgccagcagtatatgatcagatgcatttgccttga | | | |
| | | tttcccacacacaagtgatgttgtcatgcatttcattct | | | |
| | | ctgggggattcctaatcgctccagatgagttggtaat | | | |
| | | aatgccaccacaccgagatttttctggtggtgcaga | | | |
| | | agacggtgggtgttgagccactgatatcacca | | | |
| | | (SEQ ID NO:67) | | | |
| 35. | 3072856/3072 | atcgacatgacgatcgcggagatgccgggcgcgt | SVILPLLLSDSPVIAWWPFS | similar to | |
| | 856 | cggttgtcacccgctcgaccgggacgccgagacg | GPDDLASDSIGALADRRITD | gi\|3334791\| | |
| | | caatcctagccaggctgccagcagcatcgccggc | SAADKDPCKALIRRAAHLTE | hypothetical protein | |
| | | gcattaccggcggctgactctacccgagcgaactt | GDSDLCWARTTSWRALAA | SC5A7.10c | |
| | | gacggtcgctggatgttgatccaaagctgctgcag | AALDQHPATVKFARVESAA | [Streptomyces | |
| | | ctagggctctccagctggtggtgcgagcccaacac | GNAPAMLLAAWLGLRLGVP | coelicolor] | |
| | | aggtcggagtcaccctcggttaggtgagccgcac | VERVTTDAPGISAI(SEQ ID | | |
| | | ggcgtatgagggctttgcacggatctttgtcagctgc | NO:70) | | |
| | | cgagtcggtgatgcggcggtccgcaagggctccg | | | |
| | | atggagtccgaggcgaggtcgtcagggccggag | | | |
| | | aagggccaccacgcaatgacgggggaatccga | | | |
| | | cagtagcagcggaaggataaccgaggcagg | | | |
| | | (SEQ ID NO:69) | | | |
| 36. | 21425621/214 | gtacaccaaccgggcgctgacggactttcagtttgt | NRALTDFQFVLTLPFWAVE | similar to gi\|5566386 | |
| | 25621 | gctcaccttgcccttctgggcggtggagaacgctctt | NALDFKWPFGKAMCKIVSM | angiotensin type 1 | |
| | | gacttcaaatggcccttcggcaaggccatgtgtaa | VTSMNMYASVFFLTAMSVT | receptor [Cavia | |
| | | gatcgtgtccatggtgacgtccatgaacatgtacgc | RYHSVASALKSHRTR(SEQ | porcellus] | |
| | | cagcgtgttcttcctcactgccatgagtgtgacgcg | ID NO:72) | | |
| | | taccattcggtggcctcggctctgaagagccaccg | | | |
| | | gacccgaggacacggccggggcgactgctgcg | | | |
| | | gccggagcctgggggacagctgctgcttctcggcc | | | |
| | | aaggcgctgtgtgtgtggatcc (SEQ ID | | | |
| | | NO:71) | | | |
| 37. | 25177551/251 | caactgcacttctggtcaagttgtttccctcagatgct | ECGARPLASRIVGGQSVAP | similar to gi\|4210355\| | |
| | 77551 | ctgagtgtggagcgaggcccctggcttcccggata | GRWPWQANVALGFRHTCG | dJ1170K4.2 (novel | |
| | | gttggtggtcagtctgtggctcctgggcgctggccgt | GSVLAPRWVVTAAHCMHS | Trypsin | |
| | | ggcaggccaacgtggccctgggcttccggcacac | FRLARLSSWRV(SEQ ID | family protein | |
| | | gtgtgggggctctgtgctagcgccacgctgggtggt | NO:74) | with class A LDL | |
| | | gactgctgcacattgtatgcacagtttcaggctggc | | receptor domains) | |
| | | ccgcctgtccagctggcgggttca (SEQ ID | | [Homo sapiens] | |
| | | NO:73) | | | |
| 38. | 16783914/167 | tgtacaggctagtgcaattcttgaactgtaggtggat | RTKKSLKSIHLQFKNCTSLY | similar to gi\|2497254 | |
| | 83914 | ggatttcagggacttcttggtccggg (SEQ ID | (SEQ ID NO:76) | NOV PROTEIN | |
| | | NO:75) | | HOMOLOG | |
| | | | | PRECURSOR | |
| | | | | (NOVH) | |
| 39. | 20571460/205 | gatcctgccaccagcccgcgggccccgctggcg | PPNLPGNPIPACKSPHNAD | similar to gi\|81286\| | |
| | 71460 | aaccacgggcgctaggcgtggactgtccgggcg | CSQTSPWNPRPPKLFSSCP | etensin—Volvox | |
| | | gaggaggcgccccggggcaactggagaacaac | GAPPPPGQSTPSARGSPA | carteri | |
| | | tttgggggtctggggttccaaggagaagtttgggaa | GP(SEQ ID NO:78) | (fragment) | |
| | | cagtcggcgttatgcggacttttgcaggctgggatg | | | |
| | | gggtttccgggcaggtttgggggaaaaaggaagg | | | |
| | | ggcctaagaccgcgcgcggaaagatgccgttattt | | | |
| | | gg (SEQ ID NO:77) | | | |
| 40. | 10093872/100 | taacacaagttgttcctgcagctgcctccatagctg | AFYFVCNYSPKGNWIGEAP | similar to late | |
| | 93872 | ggtgggcactcagagcagggccggccattcttgta | YKNGRPCSECPPSYGGSC | gestation lung protein | |
| | | gggggcttctccaatccagttcccctttggagaata | RNNLC(SEQ ID NO:80) | 1 | |
| | | attgcagacaaagtagaacgc (SEQ ID | | [Rattus | |
| | | NO:79) | | norvegicus] | |
| 41. | 11755212/117 | actctttgactcggcggcgggcggggggcgcttgg | SYTSSQSCRRNVKYRRVQ | similar to gi\|4758632 | |
| | 55212 | gcagcggcatgaaggatgtggagtcgggccggg | NYLYNVLERPRGWAFIYHA | potassium voltage- | |
| | | gcagggtgctgctgaactcggcagccccagggg | FVFLLVFGCLISSVFSPTPE | gated | |
| | | cgacggcctgctactgctgggcacccgcgcggcc | HTKLASSCLWILEFVMIVVL | channel, KQT-like | |
| | | acgctcggtggcggcggcggtggcctgagggag | GSEFIIRIWSAGCCCRHRG | subfamily, member 4 | |
| | | agccgccggggcaagcagggggcccggatgag | WQGSLRCARKPF(SEQ ID | | |
| | | cctgctggggaagccgctctcttacacgagtagcc NO:82) | | | |
| | | agagctgccggcgcaacgtcaagtaccggcggg | | | |
| | | tgcagaactacctgtacaacgtgctggagagacc | | | |
| | | ccgcggctgggcgttcatctaccacgctttcgtttttct | | | |
| | | ccttgtctttggttgcttgatttcgtcagtgttctccccca | | | |
| | | cccctgagcacacgaaattggcctcaagttgcctct | | | |
| | | ggatcctggagttcgtgatgattgtcgtcttgggttcg | | | |
| | | gagttcatcattcgaatttggtctgcgggttgctgttgt | | | |
| | | cgacatagaggatggcaaggaagtctgaggtgtg | | | |
| | | ctcggaagcccttcccccg (SEQ ID NO:81) | | | |
| 42. | 20421338/204 | cccacaacctcacatgccgggccttcaatgcgaa | LTCRAFNAKPAATIIWFRDG | sililar to gi\|3451335 | |
| | 21338 | gcctgctgccaccatcatctggttccgggacggga | TQQEGAVASTEIAEDGKRE | F22162_1 [Homo | |
| | | cgcagcaggagggcgctgtggccagcacggaat | TTVSQLLINPTDLDIGRVFTC | sapiens] | |
| | | tgctgaaggatgggaagagggagaccaccgtga | RSMNEAIPSGKETSIELDVH | | |
| | | gccaactgcttattaaccccacggacctggacata | HPPTVTLSIEPQTGQEGER | | |
| | | gggcgtgtcttcacttgccgaagcatgaacgaagc | VVFTCQATANP(SEQ ID | | |
| | | catccctagtggcaaggagacttccatcgagctgg | NO:84) | | |
| | | atgtgcaccaccctcctacagtgaccctgtccattg | | | |
| | | agccacagacggggcaggagggtgagcgtgttg | | | |
| | | tctttacctgccaggccacagccaaccccgagatc | | | |
| | | (SEQ ID NO:83) | | | |
| 43. | 17684363/176 | agatctgggctgtggaaaatctgagacacagaag | LSYPKDFACVHQTLKAFSS | ncodes a protein in | |
| | 84363 | tgacacctttggatgaaaaagcctttagtgtctggtg | KGVTSVSQIFHSPD(SEQ ID | sequence listing 230 | |
| | | gacacaggcaaaatccttggggtaggaaag | NO:86) | that is | |
| | | (SEQ ID NO:85) | | gi\|3236326 C1 | |
| | | | inhibitor [Mus | | |
| | | | musculus] | | |
| 44. | 17584851/175 | cgatcctttggcagagtatacttgggtgttctaagaa | SFGRVYLGVLRNGRSAAVK | similar to gi\|3668069 | |
| | 84851 | atggtaggagtgccgcggtcaaaaagttggattct | KLDSNKQPDQEFLAQVSMV | Pto kinase interactor | |
| | | aacaagcagccagaccaagagttcttggcccag | SRLKHENVVELLGYCADGT | 1 | |
| | | gtgtctatggtgtcaaggctgaagcatgaaaatgtt | LRVLAYEFATMGSLHEM(SEQ | [Lycopersicon | |
| | | gtcgagttgcttggttactgtgctgatgggacactcc | ID NO:88) | esculentum] | |
| | | gcgtccttgcttatgagttcgctacaatgggttccctt | | | |
| | | catgagatgct (SEQ ID NO:87) | | | |
| 45. | 17134322/171 | ccgcgttggggagacgacggtgaccttcccagca | WLTQEAFDKLTQELEYLKG | similar to gi\|2896717\| | |
| | 34322 | agctcatcgcaggatgaaacaatccgcgccagc | RGRTVIANKIADARSEGDLS | greA [Mycobacterium | |
| | | gttaagaccttctcgcgggctgtcaccgccgatctg | ENGGYHAAREEQGQAEARI | tuberculosis] | |
| | | gagaagtgtggaccgatcaggtgacactcgcggt | RQL(SEQ ID NO:90) | | |
| | | agaatgaatagatgcctgagtctgaagacactgtg | | | |
| | | tggctgacccaagaggccttcgataagctcactca | | | |
| | | ggagctggagtacctcaaaggcgaaggccgcac | | | |
| | | cgtcattgccaacaagattgccgacgcccgttcgg | | | |
| | | aaggcgacctttctgagaacggcggctaccatgc | | | |
| | | cgcccgtgaggagcaggggcaggccgaggccc | | | |
| | | gcatccgtcaactcga (SEQ ID NO:89) | | | |
| 46. | 46872089/468 | agcaactacaggaaatggctttgggagttccaata | CLEDHNSYCINGACAFHHE | similar to gi\|4557567 | |
| | 72089 | tcagtctatcttttattcaacgcaatgacagcactga | LEKAICRCFTGYTGERCEHL | epiregulin precursor | |
| | | ccgaagaggcagccgtgactgtaacacctccaat | TLT(SEQ ID NO:92) | | |
| | | cacagcccagcaaggtaactggacagttaacaa | | | |
| | | aacagaagctgacaacatagaaggacccatagc | | | |
| | | cttgaagttctcacacctttgcctggaagatcataac | | | |
| | | agttactgcatcaacggtgcttgtgcattccaccatg | | | |
| | | agctagagaaagccatctgcaggtgttttactggtt | | | |
| | | atactggagaaaggtgtgagcacttgactttaactt | | | |
| | | catatgctgtggattcttatgaaaaatacattgcaatt | | | |
| | | (SEQ ID NO:91) | | | |
| 47. | 16530243/165 | acgcgtcctgcctcgagggcctcgggagcttccgc | ASCLEGLGSFRCLCWPGYS | similar to gi\|117422 | |
| | 30243 | tgcctctgttggccaggctacagcggcgagctgtg | GELCEVDEDECASSPCQH | CRUMBS PROTEIN | |
| | | cgaggtggacgaggacgagtgtgcatcgagccc | GGRCLQRSDPALYGGVQA | PRECURSOR | |
| | | ctgccagcatgggggccgatgcctgcagcgctctg | AFPGAFSFRHAAGFLCHCP | (95F) | |
| | | acccggccctctacgggggtgtccaggccgccttc | PGFEGADCGVEVDECASR | | |
| | | cctggcgccttcagcttccgccatgctgcgggtttcc | PCLNGGHCQDLPNGFQCH | | |
| | | tgtgccactgccctcctggctttgagggagccgact | CPDGYAGPTCEEDVDECLS | | |
| | | gcggtgtgg (SEQ ID NO:93) | DPCLHGGTCSDTVAG(SEQ | | |
| | | ID NO:94) | | | |
| 48. | 30370359/303 | gagtcaggaggcatgtaccactgccacagtggct | YHCHSGSKPTEKGANEYAY | similar to gi\|2144024 | |
| | 70359 | ccaagcccacagaaaagggggcgaatgagtac | AKWKLCSASAICFIFMIAEV | zinc transporter ZnT- | |
| | | gcctatgccaagtggaaactctgttctgcttcagca | VGGHIAGSLAVVTDAAHLLI | 2—rat | |
| | | atatgcttcattttcatgattgcagaggtcgtgggtgg | DLTSFLLSLFSLWLSSKPPS | | |
| | | gcacattgctgggagtcttgctgttgtcacagatgct | KRLTFGWHRAEILGALLSIL | | |
| | | gcccacctcttaattgacctgaccagtttcctgctca | CIWVVTGVLVYLACERLLYP | | |
| | | gtctcttctccctgtggttgtcatcgaagcctccctcta | DYQIQATVMIIVSSCAVAANI | | |
| | | ag (SEQ ID NO:95) | VLTV(SEQ ID NO:96) | | |
| 49. | 32557285/325 | caagtgatggccgtgctcctacactacttcttcctga | VMAVLLHYFFLSAFAWMLV | similar to gi\|4164075 | |
| | 57285 | gtgccttcgcatggatgctggtggaggggctgcac | EGLHLYNMVIKVFGSEDSK | latrophilin 3 splice | |
| | | ctctacaacatggtgatcaaggtctttgggtcggag | HRYYYGMGWGFPLLICIISL | variant bbbh [Bos | |
| | | gacagcaagcaccgttactactatgggatgggatg | SFAMDSYGT(SEQ ID | taurus] | |
| | | gggttttcctcttctgatctgcatcatttcactgtcatttg | NO:98) | | |
| | | ccatggacagttacggaacaag (SEQ ID | | | |
| | | NO:97) | | | |
| 50. | 30664188/306 | aagctttgcgcaacgccagcctcaggcgagatga | KGNGYVQSPRFPNSYPRNL | similar to gi\|1209014 | |
| | 64188 | gagcaatcacctcacagacttgtaccgaagagat | LLTWRLHSQENTRIQLVFD | tld protein [Xenopus | |
| | | gagacaatccaggtgaaaggaaacggctacgtg | NSVWLEEAENDICRYDFVE | laevis] | |
| | | cagagtcctagattcccgaacagctaccccagga | VEDISETSTIIRGRWCGTKE | | |
| | | acctgctcctgacatggcggcttcactctcaggaga | VPPRIKSRTNQIKITFKSDD | | |
| | | atacacggatacagctagtgtttgacaatcagtttg | (SEQ ID NO:100) | | |
| | | gattagaggaagcagaaaatgatatctgtaggtat | | | |
| | | gattttgtggaagttgaagatatatccgaaaccagt | | | |
| | | accattattagaggacgatggtgtggacacaagg | | | |
| | | aagttcctccaaggataaaatcaagaacgaacca | | | |
| | | aattaaaatcacattcaagtccgatgactactttgtg | | | |
| | | gctaaacctggattcaagatttattattctttgctgga | | | |
| | | agatttccaacccgcagcagcttcagagaccaact | | | |
| | | gggaatctgtcacaagctctatttcag (SEQ ID | | | |
| | | NO:99) | | | |
| 51. | 21648124/216 | gacaggggccctgtccacagggcgctgccgccc | ALSTGLPPLAPEGDSVASK | similar to gi\|2047300 | |
| | 48124 | ctggctccggagggcgactctgtggccagcaagc | HAIYAVQVIADPPGPGE(SEQ | latent transforming | |
| | | acgccatctacgccgtccaggtgatcgctgaccct | ID NO:102) | growth | |
| | | cctgggcccggggaggg (SEQ ID NO:101) | | factor-beta | |
| | | | binding protein [Mus | | |
| | | | musculus] | | |
| 52. | 25334288/253 | ctgcagaagttgaagccttccgctaggcacgtgag | VDGAWGPWTPWGDCSRT | similar to gi\|5725508 | |
| | 34288 | cgagcaggccttcacgccccctccccggtacgtttt | CGGGVSSSSRHCDSPRPTI | METH2 protein | |
| | | ccacttgtagaatttcccacggaaagggatgctgtc | GGKYCLGERRRHRSCNTD | [Homo | |
| | | aaattcagaacactgcacttctctgaagtcctggga | DCPPGSQDFREVQCSEFD | sapiens] | |
| | | gccagggggacagtcatccgtgttgcaggagcgg | S(SEQ ID NO: 104) | | |
| | | tgccgccttctctcacccagacagtacttgcccccg | | | |
| | | atggttggcctggggctgtcgcaatgacggctaga | | | |
| | | agaggacacgccgccgccacaggtccggctgca | | | |
| | | gtcgccccatggagtccacggcccccaggctccg | | | |
| | | tccacaccctctgggcgcgacccaaaggggaca | | | |
| | | cagacccgtttgtagcaccaccccttgtcgatggtgt | | | |
| | | gcgtctggcacagcgtgccctcggcggccgggat | | | |
| | | gctgttggtgatgcaccg (SEQ ID NO:103) | | | |
| 53. | 29674552/296 | aatccctgactccagaacgcactcagctcctctatc | SLTPERTQLLYLLAVAVVIIL | similar to Notch2 | |
| | 74552 | tccttgctgttgctgttgtcatcattctgtttattattctgct | FIILLGVIMAKRKRKHGSLW | gene product (Feline | |
| | | gggggtaatcatggcaaaacgaaagcgtaagca | LPE(SEQ ID NO:106) | leukemia virus] | |
| | | tggctctctctggctgcctgaagt (SEQ ID | | | |
| | | NO:105) | | | |
| 54. | 37446645/374 | atgcatgttcttggcttctccacacccatgttcttacctt | FLGKNCQTVLAPCSPNPCE | similar to gi\|2373395 | |
| | 46645 | gccagccaggagcacacaagcaagtataactct | NAAVCKESPNFESYTCLCA | cell surface protein | |
| | | caaaatttggtgactctttgcaaacagcagcattctc | PDGWQGKNMGVE(SEQ ID | [Mus musculus], | |
| | | acaagggtttggggaacagggagccaatactgtc | NO:108) | notch2 | |
| | | tgacaattcttgcctagaaa (SEQ ID | | | |
| | | NO:107) | | | |
| 55. | 37004299/370 | agggtgctggcggggtagagtccggcgcggccc | ERTRSKPVLTGTHPVNTTV | similar to gi\|104O45 | |
| | 04299 | aggaaccatgcccgcgtgccacgttcccacagag | DFGGTTSFQCKVRSDVKPV | fibroblast growth | |
| | | ggacccgttccaagcccgtgctcacaggcacgc | IQWLKRVE(SEQ ID NO:110) | factor | |
| | | accccgtgaacacgacggtggacttcgggggga | | receptor A1 | |
| | | ccacgtccttccagtgcaaggtgcgcagcgacgtg | | precursor—African | |
| | | aagccggtgatccagtggctgaagcgcgtggagt | | clawed frog | |
| | | acggcgccgagggccgccacaactccaccatcg | atgtgggcggccagaagtttgtggtgctgcccacg | | |
| | | ggtgatgagcagcttattgaggtaggag (SEQ | | | |
| | | ID NO:109) | | | |
| 56. | 11751803/117 | ccacaggggcccgggcgcacggccactttgtcca | SGTQGNRWHEAATLSHQP | similar to gi\|2498110 | |
| | 51803 | gcgccatggtgccgtggtatccgtcccggaggccc | GSHAQYQLLFEGLRDGYH | APICAL | |
| | | tcgaacagcagctggtactgggcatgggagccag | GTMALDKVAVRPGPC(SEQ | ENDOSOMAL | |
| | | gctggtgggaaagggtggcccnagcctcgtgcca | ID NO:112) | GLYCOPROTEIN | |
| | | gcggttgccctgggtgcctga (SEQ ID | | PRECURSOR | |
| | | NO:111) | | | |
| 57. | 25338668/253 | ccaaaggccttccgaggtttcaagccaccgcatttc | KGLPRFQATAFLNDQAFFH | similar to gi\|2497917 | |
| | 38668 | tcaacgatcaggccttcttccactacaacaacaac | YNNNSGKAEPVEPWSHVE | ZINC-ALPHA-2- | |
| | | agtgggaaggcagagcccgtggaaccctggagc | GMEDWEKESQL(SEQ ID | GLYCOPROTEIN | |
| | | catgtggaaggaatggaggactgggagaaggaa | NO:114) | PRECURSOR | |
| | | agccagctt (SEQ ID NO:113) | | (ZN-ALPHA-2- | |
| | | | GLYCOPROTEIN) | | |
| | | | (ZN-ALPHA-2-GP) | | |
| 58. | 5644863/5644 | cctagccaacacaaagttcacatctcagcctggct | LANTKFTSQPGYIGRLYGPS | similar to gi\|2498998 | |
| | 863 | acattggaaggctctatgggccctccctaccagga | LPGNLQYCLRFHYAIYGFLK | THYROID | |
| | | aacttgcagtattgtctgcgttttcattatgccatctatg | MSDTLAVYIFEENHVVQEKI HORMONE- | | |
| | | gatttttaaaaatgagtgacaccctagcagtttacat | (SEQ ID NO:116) | INDUCED | |
| | | ctttgaagagaaccatgtggttcaagagaagatct | | PROTEIN B | |
| | | (SEQ ID NO:115) | | PRECURSOR | |
| 59. | 20131408/201 | gcgcgccagggaggcgccaggggactggtactc | GSYFGEICLLTRGRRTASV | similar to gi\|3242244 | |
| | 31408 | aggggaaggaggactcaggacatgggggcttgg | RADTYCRLYSLSVDHFNAV | hyperpolarization- | |
| | | gatgctatctctcctgggaaggacatgctgagggat | LEEFPMMRRAFETVAMDRL | activated cation | |
| | | agggacagtgggccacagtgttgaaagtcctgtta | LRIGE(SEQ ID NO:118) | channel, HAC3 | |
| | | gggagcatttgttgggactgaatggattgggatatg | | [Mus musculus] | |
| | | accctggagccctaaagcctgggctccttgctttgg | | | |
| | | aggtggggtgagagcacagtctggacccagagc | | | |
| | | agatggggtggacaggtctcaccgatgcggagca | | | |
| | | gccgatccatggccacagtctcaaaggcccggcg | | | |
| | | catcatggggaactcctcaagcacagcattgaaat | | | |
| | | ggtccacgctgagtgagtaaaggcggcagtaggt | | | |
| | | gtcagcccgaacactggctgtgcgccggccccta | | | |
| | | gttagcaggcagatctccccaaagtaggatcc | | | |
| | | (SEQ ID NO:117) | | | |
| 60. | 30674971/306 | ctggagccccagcattacctttccttcatggaatttg | MEFEITFRPDSGDGVLLYSY | similar to gi\|2988422 | |
| | 74971 | agatcacatttcggccagactcaggagatggtgtc | DTGSKDFLSINLAGGHVEF | agrin precursor | |
| | | ctcctgtacagctatgacacaggcagcaaagactt | RFDCGSGTGVLRSEDPLTL | [Homo | |
| | | cctgtccatcaacttggcagggggccacgtggagt | GNWHELRVSRTAKNGILQV | sapiens] | |
| | | tccgctttgactgtggctctgggaccggtgtcctcag | DKQKIVEGMAEGGFTQIKC | | |
| | | gagtgaagatcccctcaccctgggcaactggcac | NTDIFIGESP(SEQ ID | | |
| | | gagcttcgtgtatctcgcacagcaaagaatggaat | NO:120) | | |
| | | cttacaggtggataagcagaagatagtggaggga | | | |
| | | atggcagagggaggcttcacacagattaagtgca | | | |
| | | acacagacattttcattggcgagtcccca (SEQ | | | |
| | | ID NO:119) | | | |
| 61. | 3928094/3928 | ctgatgcccgtacacacgaaccaggtcgcgaccg | TLSSTLTSLLTSVLTVLGVLT | similar to gi\|1723082 | |
| | 094 ctgtagttctcctcgatctgtgcgttgatcttgcccgtct | MMFVISPMLALIALIAVPVSV | HYPOTHETICAL | | |
| | | cggcccactgagccttgtacaacacctgtgctcgct | LLVARVaKRAQVLYKAQWA | ABC | |
| | | tggcgaccctggcaacgaggagcaccgataccg | ETGKINAQIEENYSGRDLVR | TRANSPORTER | |
| | | gcaccgcgatcagtgcgatcagggcgagcatcg | VYGHQ(SEQ ID NO:122) ATP-BINDING | | |
| | | gagagatgacgaacatcatcgtcaggacgccga | | PROTEIN CY50.10 | |
| | | ggacggtgagcaccgacgtcaggagcgaggtca | | | |
| | | acgtcgacgacagcgtc (SEQ ID NO:121) | | | |
| 62. | 20940659/209 | ccagggtccagatcccaacggcttccgctgctact | DPNGFRCYCVPGFQGPRC | similar to gi\|117422\| | |
| | 40659.0.38 | gcgtgccgggtttccagggcccacgctgcgagctg | ELDIDECASRPCHHGATCR | CRUMBS PROTEIN | |
| | | gacatcgatgagtgtgcatcccggccgtgccacca | NLADRYECHCPLGYAGVTC | PRECURSOR | |
| | | tggggccacctgccgcaacctggccgatcgctac | ETEVDECASAPCLHGASCL | (95F) | |
| | | gagtgccattgcccccttggctatgcaggcgtgacc | DGVGSFRCVCAPGYGGTR | | |
| | | tgcgagacggaggtggacgagtgcgcctcagcg | CQLDLDECQSQPCAHGGT | | |
| | | ccctgcctgcacggggcctcgtgcctggacggcgt | CHDLVNGFRCDCAGTGYE | | |
| | | gggctccttccgctgtgtgtgcgcgccaggctacgg | GTHCEREVLECAS(SEQ ID | | |
| | | gggcacccgttgccagctggacctcgacgagtgc | NO:124) | | |
| | | cagagccagccgtgcgcacatgggggcacgtgc | | | |
| | | cacgacctggtcaacgggttccggtgcgactgcgc | | | |
| | | gggcaccggctacgagggcacgcactgcgagc | | | |
| | | gggaggtgctggagtgcgcatcggc (SEQ ID | | | |
| | | NO:123) | | | |
| 63. | 2535440/2535 | cgatcatggtggcagccgccctggggsagccgtc | LLLLGVMIGYFVSSGVTVLL | similar to gi\|5006425\| | |
| | 440 | gtgctgctcatcgtcatgctggcaggacgattcatc | SRASPELIAQYTRWQFGSY | HmuU | |
| | | cactcctcgacgatcctgctgctgctcggcgtcatg | HGVTWQNLRVMVPIIVAMIL | [Corynebacterium | |
| | | atcgggtacttcgtctcgtctggggtcaccgtactgc | ASLLLAKPLNALLLGERYAQ | diphtheriae] | |
| | | gtcgcgggcgagcccagagctcattgcccagtac | TMGMNLKVVRTLLVASTAI | | |
| | | acccgctggcagttcggcagctaccacggtgtgac | (SEQ ID NO: 126) | | |
| | | gtggcagaacctgcgagtcatggtgccgatcattgt | | | |
| | | ggccatgatcctcgccagcctgctgctcgccaagc | | | |
| | | cgcttaacgccttgttgctgggggaacgttacgctc | | | |
| | | agacgatggggatgaacctcaaggtggtgcgcac | | | |
| | | cctgctggtggcgagcacagccatc (SEQ ID | | | |
| | | NO:125) | | | |
| 64. | 21433117/214 | ccgcagattctggaggagtgccgatggccgagttc | MAEFIYTMHNVRKAVGDKVI | similar to gi\|2791517 | |
| | 33117 | atctacaccatgcacaacgtccgaaaggcggtgg | LDNVTLSFFPGAKIGVVGPN | hypothetical protein | |
| | | gtgacaaagttatccttgacaatgtcacgctgtcgtt | GAGKSTMLKLMAGLDKPNN | Rv2477c | |
| | | cttcccgggcgccaagattggtgttgtcggaccga | GDANLAKGATVGILLQEPPL | [Mycobacterium | |
| | | atggcgctggcaaatcgacgatgctcaagctcatg | TEDKTVRENVEEAVGDIKA | tuberculosis] | |
| | | gctggtctcgataagcccaataacggcgatgcca | KLARFEEVSAEMANPDARL | | |
| | | acttggctaaaggcgccaccgtcggaatcttgcttc | DALMAEMGELQTELDNANA | | |
| | | aggagcccccgctcaccgaggacaaaactgttcg | (SEQ ID NO:128) | | |
| | | cgagaacgtcgaagaggccgtcggcgacatcaa | | | |
| | | agccaagctggcacggttcgaggaagtctccgcc | | | |
| | | gagatggccaaccctgacgccgactttgacgccct | | | |
| | | gatggcggagatgggtgagctgcagaccgagctc | | | |
| | | gataacgccaacgcgc (SEQ ID NO:127) | | | |
| 65. | 8485858/8485 | acgcgtgatcgtaccatcgccgtactgcttcgcatc | LCRARSSDPHTPRCGCLHC | similar to gi\|1419019 | |
| | 858 | gagcttgtagttgtgccgagcgcgttcgtcagaccc | GSRRMSSPSCCHRSGWSP | smaphorin F [Mus | |
| | | gcatactccacggtgcggctgcctgcattgcggttct | (SEQ ID NO:130) | musculus] | |
| | | cggcgaatgtcgtcgccgtcttgttgtcaccgtagc | | | |
| | | ggctggtcacccccgtcgtattcagattgccgacac | | | |
| | | tgaggctgtccgcattcaaaaccgccttgttca | | | |
| | | (SEQ ID NO:129) | | | |
| 66. | 17705528/177 | gccatcgttcagagtgtcaagaacaagcctctcttc | AIVQSVKNKPLFFADKLHKS | similar to gi\|1351943 | |
| | 05528 | tttgctgataaactgcacaagtccatgaagggtgct | MKGAGTDEKTLTRVMVSRS | ANNEIN VI | |
| | | ggcacagatgagaagaccctcaccagggtgatg | EIDLLNIR(SEQ ID NO:132) | (LIPOCORTIN VI) | |
| | | gtgtctcggagtgagatagatctgctcaacatccgg | | (P68) (P70) | |
| | | a (SEQ ID NO:131) | | (PROTEIN III) | |
| | | | (CHROMOBINDIN | | |
| | | | 20) (67 KD | | |
| | | | CALELECTRIN) | | |
| | | | (CALPHOBINDIN-II) | | |
| | | | (CPB-II) | | |
| 67. | 2959314/2959 | cgcgtgtcccttggatacttgtcggaggtggagcga | RVSLGYLSEVERGQKEASS | similar to gi\|3294243 | |
| | 314 | ggccagaaagaagcttctagcgagcttatctcggc | ELISAICTALDLPQSDLMRIV | hypothetical protein | |
| | | catctgtactgctctggatcttcctcagtccgatctcat | SDKM(SEQ ID NO:134) | SC7C7.10 | |
| | | gcgcattgtcagtgacaagatgctcaagaacgaa | | [Streptomyces | |
| | | gacgccccggaacatcgcatcactgttgcggcttg | | coelicolor] | |
| | | aacgtaccaagcttgtcaggttcccggtcccgggg | | | |
| | | gccgggaacgtctacatttagacaagacagcgga | | | |
| | | tagttagcgccctagcgggtggctgaggggccgtc | | | |
| | | agtcgataggcgcgtggacgattcgaatcgcgata | | | |
| | | ccaagcgcgcgcagccgggctagttcttcggggtc | | | |
| | | agcagagtcgtctgtaatgaggaggtcga (SEQ | | | |
| | | ID NO:133) | | | |
| 68. | 20615755/206 | cgcgtggtcgatcgccatggtgctggctatcacctt | SIAMVLAITLLPFSIIGPFISV | similar to gi\|4808394 | |
| | 15755 | gctgcccttcagcatcattggccccttcatttccgtcg | VLDRWSRQRILVYTDGLRC | ) putative membrane | |
| | | tactcgaccgctggtcgcgtcagcgcatcctcgtct | LIAVGLGILVWNGARDTLPL | protein | |
| | | ataccgatggtctgcgctgcctcattgcggtcgggc | ALLIGLLIAMSLNRFLLTALV | [Streptomyces | |
| | | tggggatcttagtctggaatggcgcgcgcgataaa | AGLEYTIDKRDYLTASSIMP | coelicolor] | |
| | | ccctcccacctggccttgctcatcgggttactcatcg | TIGPLGLMIGAVVAAAVRMI( | | |
| | | cgatgagcctcaaccggttcttgctcaccgctttggt | SEQ ID NO:136) | | |
| | | cgctgggttggaatacaccatcgacaagcgtgact | | | |
| | | acctgacggcgtcgtcgatcatgccgacgattggc | | | |
| | | ccactcgggctcatgattggtgcggttgttgctgccg | | | |
| | | cagtgcgcatgattgccggacgccatatccctgtgc | | | |
| | | a (SEQ ID NO:135) | | | |
| 69. | 10090583/100 | caagacttttggtattggtggacttccgattacgacta | LANNFNMDEISDIVFRVNDT | similar to gi\|2633808 | |
| | 90583 | atatttctcttgccaacaacttcaatatggatgaaattt | SLTPTVGPELARsLTEIAGL | | |
| | | ctgatattgtcttccgtgtcaatgataccagtttgaca | QQREYQVSDATAAFQEVQ | | |
| | | ccaactgtgggaccagaattagctagaaaattgac | QLFGFITTIISAIAGISLFVGG | | |
| | | cgaaattgctggtcttcagcaaggggagtatcagg | TGVMNIMLVSVTERTR(SEQ | | |
| | | tgtcagatgcgactgcagccttccaagaagtgcaa | ID NO: 138) | | |
| | | caattgttcggctttataactacgattattagtgccatt | | | |
| | | gcaggaatttccctttttgttggagggactggtgttat | | | |
| | | gaacatcatgctggtttcggtgacggagcgtacgc | | | |
| | | gg (SEQ ID NO:137) | | | |
| 70. | 10186453/101 | acgcgtgtgccggggtttctggctgtctgcaggcat | IPLASEASIGFPLVGSTVFFR | similar to gi\|1685117 | |
| | 86453.0.1 | gaggtatacattcggtctgtatcccactccatgttaa | CRKGYHIQGSTTRTCLANL | furrowed | |
| | | attggcaaggcaggtgcgagtcgtggaaccttga | TWSGIQTECIPHACRQPET | | |
| | | atatggtagccttttctgcacctgaaaaaaaccgtg | PAHA(SEQ ID NO:140) | | |
| | | cttccaacctagagaggaaatccaatactagcttc | | | |
| | | agaggccaagggaatatacgaagtgtcaactagt | | | |
| | | gatacctgaaagtacacgcccgagtaaaagtaca | | | |
| | | gcagcaacttttcaggttaatgtcaagccactgtcta | | | |
| | | agtcaacatgtacattttaactgcctgctacagtgtta | | | |
| | | cacacaatgtagattgctcaatgaataatgtaagat | | | |
| | | cttctcgaattttgaaaatttagacggctgtttgggta | | | |
| | | aagaaattatgccttttctgttttagagttctttcttttttgt | | | |
| | | ttgtaataattattatttcctttttcatttttttcctgccactc | | | |
| | | acatttgaaacaactggtatttattagaaaattactttt | | | |
| | | gttagcattccaaggggattggtcaactcctactcat | | | |
| | | acagagtccaccaaaactctacct (SEQ ID | | | |
| | | NO:139) | | | |
| 71. | 21643678/216 | tggcttagcgcgcggtggcgtccgtattcgtcaagg | LLIFPEGTRSRTGAMGTFKP | similar to gi\|2983494 | |
| | 43678 | gttggcatgccgtaggcgcgggcggtttggtcgtgc | GAAALAISRGVPVIPIALVGA | long-chain-fatty-acid | |
| | | aactcaatgacctggcgacgaatccgttcggaga | WAA(SEQ ID NO:142) | CoA | |
| | | attggtgggcgatctcgcgggaacagggtccatag | | ligase [Aquifex | |
| | | ggtgtccaatagccacgtggaccaatgggcgtcct | | aeolicus] | |
| | | tttggtaacctggcttgctcggacggcatagccgcc | | | |
| | | catgctcctactaaagcaatcgggataaccggaa | | | |
| | | ccccacgtgaaatagccaatgcggcagccccag | | | |
| | | gtttgaaggtgcccattgcgccggtgcgagaccgg | | | |
| | | gtgccctccggaaagatcagcagggggacgc | | | |
| | | (SEQ ID NO:141) | | | |
| 72. | 10344740/103 | cggcagacgcaacatggcgtggcgcagcaggg | SWAPLAAHCASLLAEARTQ | similar to gi\|3483045\| | |
| | 44740 | gagcagagagcgccggcagcaggtagtgggtca | PYIRMLPVLGVGRWRTLTH | putative transport | |
| | | gcgtgcgccatcggccgacgcccaataccggca | YLLPALSAPLLRHAMLRLP | system | |
| | | acatgcggatatagggctgcgtgcgggcttccgcc | (SEQ ID NO:144) | permease protein | |
| | | aacaacgaagcacaatgggcggccagcggagc | | [Streptomyces | |
| | | ccaact (SEQ ID NO:143) | | coelicolor] | |
| 73. | 11395897/113 | ccgcgttgaccagttcctgccacggcgtaatcgcc | GLPAPVGMLFVAVLVKLCN | similar to gi\|1783249 | |
| | 95897 | acgccaacggcgaacagaatcggataggtgacg | GASPRLLEGSQVVYKFFQT | (D83026) | |
| | | gaggtctggaagaatttgtaaaccacctgcgagcc | SVTYPILFAVGVAITPWQEL | homologous to | |
| | | ttcgagcaggcggggagaagcgccgttgcacagt | VNA(SEQ ID NO:146) | citrate-sodium | |
| | | ttgaccagcaccgccacaaacaacatgccaacc | | symport (citrate | |
| | | ggagcaggcaggccaatca (SEQ ID | | transporters); | |
| | | NO:145) | | hypothetical | |
| | | | [Bacillus subtilis] | | |
| 74. | 17937351/179 | cgatcttctggtcggcggtgatcacgctggtgacca | IFWSAVITLVTIGLLFAGNFE | similar to gi\|114921\| | |
| | 37351 | tcggcctgctgtttgccggcaacttcgaagccatgc | AMQTMVVLAGLPFSVVLIFF | HIGH-AFFINITY | |
| | | aaaccatggtcgtgctggccgggctgccgttctcg | MFGLHKAMR(SEQ ID | CHOLINE | |
| | | gtggtgctgattttcttcatgttcggtttgcacaaggcg | NO:148) | TRANSPORT | |
| | | atgcgccaggacgtggccatggagcaggagcag | | PROTEIN | |
| | | gcacaattggctgaacgtggtcgccgtggtttcagc | | | |
| | | gagcgcctgaccgcgctggacctgcaaccgagc | | | |
| | | cagggcaccgtgcaacgctttatggacaaacatgt | | | |
| | | gacgccggcgttggaacaagcggcgactgcgttg | | | |
| | | cgtgatcaag (SEQ ID NO:147) | | | |
| 75. | 19895736/198 | cgcgtcgcctcctgctggtcgggattttccttgctgta | RRLLLVGIFLAVVNQTTGVN | similar to gi\|1750127 | |
| | 95736 | gttaaccaaaccaccggcgtcaataccgtcatgta | TVMYYAPKVLEFAGMSTQA | YncC [Bacillus | |
| | | ttacgcgcccaaggtgttggagttcgcaggaatga | SIISEVANGVMSVIGAAAGL | subtilis] | |
| | | gcacccaggcgtcgattatttcagaggtggctaatg | WLIERFDRRHLLIFDVTAVG | | |
| | | gagtcatgtctgttattggtgccgctgcaggcttgtgg | VCLLGIA(SEQ ID NO:150) | | |
| | | ctcatcgaacggtttgatcgtcgtcacctgcttatcttc | | | |
| | | gatgtcacggcggtcggtgtgtgtctccttggtattgc | | | |
| | | ggctactttcgggctggcaattgctcctcatgtgggt | | | |
| | | caaggggtaccgaagtgggcgcctattctcgtgct | | | |
| | | cgtcctgatgagtatcttcatgcttatcgtgca | | | |
| | | (SEQ ID NO:149) | | | |
| 76. | 20370351/203 | tccgcactgtggcgaccatccttgccaccattacca | GEDEVSRKLITVWGAEPQN | similar to gi\|2960090\| | |
| | 70351 | ttgccgccctagtgctcacgggctgtaatacggcg | PLLPADTNETGGTKVITALF | dppA | |
| | | gtgcgccaaacggtgaagacgaggtttcccgcaa | AGLVYYDADGKTHNDVAKS | [Mycobacterium | |
| | | gctcatcaccgtgtggggtgctgagccacaaaac | IDFDGDRTYTVTLR(SEQ ID | tuberculosis] | |
| | | ccactcctgccagccgacaccaatgaaaccggc | NO:152) | | |
| | | ggcacgaaagtcatcaccgccttgttcgccggcct | | | |
| | | ggtgtattacgacgccgacggcaaaacccataat | | | |
| | | gatgtggccaaatccattgacttcgatggcgaccg | | | |
| | | cacctacacggtgacgctgcggaaaaccagattc | | | |
| | | gccg (SEQ ID NO:151) | | | |
| 77. | 20438222/204 | aatttaataccatagccttctcttggttgatccttctag | IAFSWLILLGMSYGIKTGIHL | similar to gi\|2224836\| | |
| | 38222 | gcatgagttatggcattaaaacgggcatccatcttg | GVDIVLNAVPKRVSRALSLF | small integral | |
| | | gtgtcgatatcgtacttaatgccgtgcctaaacgagt | GAFAAIMY(SEQ ID NO:154) | membrane | |
| | | atcaagagccttgtctttgttcggtgcctttgccgctat | | transport protein | |
| | | tatgtacggtctcattctacttgattctacctggttagc | | [Rhodobacter | |
| | | cttactcggtatcgatgtacgaggtggtgccatcga | | capsulatus] | |
| | | atattgggcgaagatgttcaaaataggtattggtact | | | |
| | | gaagagcttcgttaccctatctttatgcaagatatgttt | | | |
| | | gatttgcgcccacgcg (SEQ ID NO:153) | | | |
| 78. | 8504426/8504 | ccatcctcccaccatctcttccctcactccctcatcca | PPTISSLTPSSNIARKKTLPP | similar to gi\|4512604 | |
| | 426 | acattgccagaaaaaaaacgctcccaccaccctt | PFTLQELQVPLIDAKTCNTY | mastin precursor | |
| | | caccctgcaggagttgcaggtgcctcttattgatgc | YQ(SEQ ID NO:156) | [Canis sp.] | |
| | | caagacctgcaatacctactaycaggagaactcc | | | |
| | | (SEQ ID NO:155) | | | |
| 79. | 4773473/4773 | tgcacccgcagcgagcgttttgcggtgcgggtgtg | HPQRAFCGAGVVVRAKAV | similar to gi\|4929928\| | |
| | 473 | gtggtcagggccaaagcggtcagtggggggggg | SGGGVGSGGGVCGGPV(SEQ | | |
| | | gtgggctctgggggcggcgtttgtggcggccctgtc | ID NO:158) | | |
| | | gggggggtc (SEQ ID NO:157) | | | |
| 80. | 8483814/8483 | gatcccaggaggaggatcaggaccatgatggac | NSCSSFIYGGCRGNKNSYL | similar to gi\|4539685\| | |
| | 614 | agccccaccaggatcaccgcttttgtgcccggggt | SQEACMQHCSGKQMYPFL | hepatocyte growth | |
| | | caggaaaggatacatctgcttcccagagcaatgct | TPGTKAVILVGLSIMVLILLL | factor | |
| | | gcatgcacgcttcctgggagaggtaactgttcttgtt | G(SEQ ID NO:160) | activator inhibitor | |
| | | gcctctgcacccgccatagatgaagctgctacagg | | type 2 splice variant | |
| | | aattg (SEQ ID NO:159) | | 1 [Mus | |
| | | | musculus] | | |
| 81. | 20695553/206 | caattccagtttctccatgattgacagcatgtcttggc | VPQSPTHQLEDMEKALNPT | similar to gi\|3236326 | |
| | 95553 | tgctctttactttaatgcgaggcatcatcacgtaagtg | VFKAILKKLGLSKFQPTYVM | C1 inhibitor [Mus | |
| | | ggctgaaacttggacagccctagcttcttcaggatg | MPRIKVKSSQDMLSIMEKLE | musculus] | |
| | | gccttaaagacagtggggttgagtgccttttccatgt | (SEQ ID NO:162) | | |
| | | cttcaagttggtgtgtcgggctctggggtact(SEQ | | | |
| | | ID NO:161) | | | |
| 82. | 18598551/185 | cgagggcttccctctgaataaagacgacatagac | EGFPLNKDDIDGIQYLYGRG | 100% similar to | |
| | 98551 | ggcatccagtatctgtatggtcggggctctaagcct | SKPDPRPPATTTTEPQPTA | gi\|433435 gelatinase | |
| | | gacccaaggcctccagccaccaccacaactgaa | PPTMCPTIP(SEQ ID | b [Mus | |
| | | ccacagccgacagcacctcccactatgtgtcccac | NO:164) | musculus] | |
| | | tatacct (SEQ ID NO:163) | | | |
| 83. | 29017225/290 | gaattctccaggatagtctggaggtggtgataccat | DLSRFNFDNKYGRNALEIVV similar to gi\|2078282 | | |
| | 17225 | aggagaatccaagtttacaatggatttcacgacaa | KSIVNLDSPMVSPPPDYPG | Sno [Drosophila | |
| | | tttctaaagcatttcttccatacttattatcaaagttgaa | EF(SEQ ID NO:166) | melanogaster] | |
| | | cctgctcagatct (SEQ ID NO:165) | | | |
| 84. | 29187133/291 | cggaggcccactggtttgcaaggagcccagtggt | GGPLVCKEPSGRWFLAGLV | similar to gi\|4210355\| | |
| | 87133 | cgctggttcctggcagggttggttagctggggcctg | SWGLGCGRPNFFGVYTRV | dJ1170K4.2 (novel | |
| | | ggttgtggccgacccaatttctttggtgtctacactcg | TRVVNWI(SEQ ID NO:168) | Trypsin | |
| | | tgtcacacgtgtggtcaactggatcc (SEQ ID | | family protein | |
| | | NO:167) | | with class A LDL | |
| | | | receptor domains) | | |
| | | | [Homo sapiens] | | |
| 85. | 2876041/2876 | ccgcgtcgacgaccatgacggcaccgacggagt | LRWFDITDDGRLDLREAEA | similar to gi\|5459388\| | |
| | 041 | gggcctgctcggcaatcttcccgatgggattgatcg | EGLINERTKVVSLTLASNVL | putative | |
| | | tccccagcacgttcgaggccaacgtcaacgagac | GTINPIGKIAEQAHSVGAVM | aminotransferase | |
| | | gaccttggtgcgctcattaatgagcccctccgcctc | VVDA(SEQ ID NO:170) | [Streptomyces | |
| | | agccttctcgaggtcgaggcggccgtcatcagtga | | coelicolor] | |
| | | tgtcgaaccacctcag (SEQ ID NO:169) | | | |
| 86. | 2960355/2960 | gtcgaccacggtgacgactgacaagaatccctcct | RVMPIHGEVRLVANADLAK | similar to gi\|3413828\| | |
| | 355 | caccgaggatacggcgatcagtgagcgtgtcctc | ATGVDENNVVLVEDGGVID | hypothetical protein | |
| | | ggtaagctcccccaccccanatccgtcaacaagg | LVDGVPRVVGKVDASYILV | SC9A10.09 | |
| | | atgtacgaggcatcgaccttgccaacaactcgcg | DGGVGELTEDTLTDRRILG | [Streptomyces | |
| | | gtactccgtcaacaagggtcaataaccccgccgtc | EEGFLSVVTVVD(SEQ ID | coelicolor] | |
| | | ctcgacaagcaccacgttgttctcatcgacaccggt | NO: 172) | | |
| | | tgctttggccagatcggcattagcgacaagangac | | | |
| | | gcacctcaccatgaatcggcatcacancgcgngg | | | |
| | | gcccncnccgaagttntaccccgntgcatgggat | | | |
| | | nttcacgcgggaanccnaatttca (SEQ ID | | | |
| | | NO:171) | | | |
| 87. | 2963495/2963 | cgcgtcaggcccacgtagtttcttggtgagcttatag | SVTGSSSHPTGTGVGAALF | similar to gi\|2808789\| | |
| | 495 | aaggcgtacccagcccacggtcccgcgatagcc | KPPVMAFLGMIVLIFQALLLA | putative cobalt | |
| | | atcgagaaagtgttggctcccagggtggagattcc | HGGISTLGANTFSMAIAGP | transport | |
| | | cccgtgagccaggagcagggcctggaagatgag | WAGYAFYKLTKK(SEQ ID | protein | |
| | | cacgatcatccccaggaaggccatgacaggcgg | NO:174) | [Streptomyces | |
| | | tttgaacagtgccgccccaactcccgttccggtggg | | coelicolor] | |
| | | gtgcgaggacgatcccgtaacgctgg (SEQ ID | | | |
| | | NO:173) | | | |
| 88. | 10076564/100 | gatgtcggtgttctcgtcgtccacgtcttcgtcgtcaa | YADSTAVVVGPLAPAPDPHA | similar to | |
| | 76564 | gctcgacgtgctcgacgcgaaccagctcccaccc | WDLCERHSAHITAPVGWEL | gi\|2894206\| | |
| | | caccggcgctgtgatgtgggcggagtggcgctcg | VR(SEQ ID NO:176) | hypothetical protein | |
| | | cacaagtcccaggcgtgggggtcgggcgcgggc | | Rv3258c | |
| | | gcaagcgggccgaccactgccgtcgagtccgcgt | | [Mycobacterium | |
| | | acgcgt (SEQ ID NO:175) | | tuberculosis] | |
| 89. | 16399653/163 | cgatcactcccttgtgtaccggggccgtcaccatgg | AATEAGQLDAANGRYVLAT | similar to gi\|216477\| | |
| | 99653 | cggcaaattcgccgctctggcagccgtctatcgcc | LDVAIDGCQSGEFAAMVTA | pdxA protein | |
| | | acatccagcgtagccagtacatagcggccgttgg | PVHKGVI(SEQ ID NO:178) | [Escherichia | |
| | | ccgcatccagctgccctgcctcggtggctgcggcc | | coli] | |
| | | a (SEQ ID NO:177) | | | |
| 90. | 11076711/110 | gatgtataccaagttggccagtccgagagtgacttc | QAIMTGFERRYGVLERLSA | similar to | |
| | 76711 | agctttgccagcgccggccattgccagtgctgcga | TPLGRSGLLAGKAMAYSVIS | gi\|5459396\| putative | |
| | | gcccgaatgtcatcatggcgagcacaacgctcac | LAQVILLVIVSLALGWHPHG | integral membrane | |
| | | cagggttgggagccaggccaggccggaaccgtg | SGLALAPTLVSVVLAMMTF | transport protein | |
| | | ggggtgccagcccagcgctaaagagacgatgac | GLAALAMAGAGKAEVTLGL | [Streptomyces | |
| | | aagcagtatcacctgagcgagactgataacggaa | ANLVYI(SEQ ID NO:180) | coelicolor] | |
| | | taagccatcgccttgccagctagcagacccgacc | | | |
| | | gacctaacggggttgcggacaatcgttcgagcac | | | |
| | | cccgtaacgacgttcaaaaccggtcatgatcgctt | | | |
| | | gggaag (SEQ ID NO:179) | | | |
| 91. | 11816129/118 | ccagaggccgaggccctgcgcttccagctcgcta | EENEVGCPEGFELDSQGAF | similar to gi\|3581785\| | |
| | 16129 | cagccctgcaggcggaggagaacgaggtcggct | CVDVDECLEGLDDCHYNQL | FIBULIN 1 like | |
| | | gccccgagggctttgagctggactcccagggagc | CENTPGGHRCSCPRGYRM | protein | |
| | | gttttgtgtggatgtggacgaatgcctggaggggttg | QGPSLPCLDVNECLQLPKA | [Homo sapiens] | |
| | | gacgactgtcactacaaccagctctgcgagaaca | CAYQCHNLQGSYRCLCPP | | |
| | | ccccaggcggtcaccgctgcagctgccccaggg | GQTLLRDGKACTSL(SEQ ID | | |
| | | gttaccggatgcagggccccagcctgccctgccta | NO:182) | | |
| | | gatgtcaatgagtgcctgcagctgcccaaggcctg | | | |
| | | cgcctaccagtgccacaacctccagggcagctac | | | |
| | | cgctgcctgtgccccccaggccagaccctccttcg | | | |
| | | cgacggcaaggcctgcacctcactggagcggaa | | | |
| | | tggacaaaatgtgaccaccgtcagccaccgaga | | | |
| | | ccctctattgccctggctgcggccctgggcctcgat | | | |
| | | ccccggtacc (SEQ ID NO:181) | | | |
| 92. | 4035948/4035 | cctagcaataagccaagtagcagagctccggaat | TSILVTVFFIVLCANAVNFID | similar to | |
| | 948 | cacccatgaacatcctcgccggatgccagttgtgg | GLDGLASGVVAIGSLAFFSY | gi\|1710106 | |
| | | ggcaaaaaaccgagacaggcgcccgccgtcgc | TYLLAHEQDFVVATTTSLIT | PUTATIVE | |
| | | agccgtaatgagactggtagtcgtcgcaacaaca | AATAGACLGFLPHNWHPAR | | |
| | | aagtcctgttcgtgagccagcaggtaggtgtatga | MFMGDSGALLLGLLL(SEQ | UNDECAPRENYL- | |
| | | gaagaaagccaaggacccgatggccaccacac | ID NO: 184) | PHOSPHATE | |
| | | cggatgccaggccgtcaagtccatcaatgaaattc | | ALPHA-N- | |
| | | accgcattggcgcacaacacaatgaagaacacc | | ACETYLGLUCOSAM | |
| | | gtcaccaagatcgaagtaggcgtc (SEQ ID | | INYLTRANSFERASE | |
| | | NO:183) | | | |
| 93. | 21645656/216 | ccggtcccctgcgggatgaggtacctgatgggag | GPLRDEVPDGSRRHEQKLE | similar to gi\|2l37458\| | |
| | 45656 | ccggagacacgaacagaagctagaggcagcag | AAAQEDSPAPPSPSLQPDP | interleukin-11 | |
| | | ctcaggaggacagccccgctcctccaagcccttcc | RPL(SEQ ID NO:186) | receptor | |
| | | ttgcagccagacccaaggccacttga (SEQ ID | | alpha-chain | |
| | | NO:185) | | precursor—mouse | |
| 94. | 20396935/203 | ccgcgtgagcgtcgacaatgctccaggaaccggt | RVSVDNAPGTGVYEGVDST | similar to gi\|5639946\| | |
| | 96935 | gtgtatgaggccgtggattctaccggtcgtggtttgc | GRGLQGMRERARIHGGTA | histidine kinase CstS | |
| | | agggcatgcgtgagcgcgcccgtatccatggcgg | RWGDSQYYEGGFNVTVEIP | [Corynebacterium | |
| | | caccgcgcgctggggcgactcgcagtattatgaa | (SEQ ID NO:188) | diphtheriae] | |
| | | ggcggtttcaacgtcacggtggagattccaacatg | | | |
| | | agcggccaaaggatgaacatggacacgacgcg | | | |
| | | cccaatcacggtcggggcttgccgacgatcagcc | | | |
| | | ggctg (SEQ ID NO:187) | | | |
| 95. | 4016127/4016 | atcgacatgacgatcgcggagatgccgggcgcgt | KDKLDAEVHAGEGTPGDVI | similar to gi\|3334791\| | |
| | 127 | cggttgtcacccgctcgaccgggacgccgagacg | VLRFSGAMAKRPASVILPLL | hypothetical protein | |
| | | caatcctagccaggctgccagcagcatcgccggc | LSDSPVIAWWPFSGPDDLA | SC5A7.10c | |
| | | gcattaccggcggctgactctacccgagcgaactt | SDSIGALADRRITDSAADKD | [Streptomyces | |
| | | gacggtcgctggatgttgatccaaagctgctgcag | PCKALIRRAAHLTEGDSDLC | coelicolor] | |
| | | ctagggctctccagctggtggtgcgagcccaacac | WARTTSWRALAAAALDQH | | |
| | | aggtcggagtcaccctcggttaggtgagccgcac | PATVKFARVESAAGNAPAM | | |
| | | ggcgtatgagggctttgcacggatctttgtcagctgc | LLAAWLGLR(SEQ ID | | |
| | | cgagtcggtgatgcggcggtctgcaagggctccg | NO:190) | | |
| | | atggagtccgaggcgaggtcgtcagggccagag | | | |
| | | aagggccaccacgcaatgacgggggaatccga | | | |
| | | cagtagcagcggaaggataaccgaggcagggc | | | |
| | | gcttcgccatggctccggaaaaccgcagcacgat | | | |
| | | gacatccccgggggtgccttcaccggcatgcacct | | | |
| | | cggcgtcaagcttgtccttacgagaacgcg | | | |
| | | (SEQ ID NO:189) | | | |
| 96. | 3941385/3941 | acgcgtgcggctgctgcgggagctcagcgagcg | LELVFLVDDSSSVGEVNFR | similar to gi\|323091\| | |
| | 385 | cctggagcttgtcttcctggtggatgattcgtccagc | SELMFVRKLLSDFPVVPTAT | immunodominant | |
| | | gtgggcgaagtcaacttccgcagcgagctcatgtt | RVAIVTFSSKNYV(SEQ ID | microneme | |
| | | cgtccgcaagctgctgtccgacttccccgtggtgcc | NO:192) | protein Etp100— | |
| | | cacggccacgcgcgtggccatcgtgaccttctcgt | | Eimeria tenella | |
| | | ccaagaactacgtggtgccgcgcgtcgattacatc | | | |
| | | tccacccgccgcgcgcgccagcacaagtgcgcg | | | |
| | | ctgctcc (SEQ ID NO:191) | | | |
| 97. | 10196003/101 | ccgcgtgggctgatcggcatgctctgggcactggg | IGMLWALGVVAEVLMFLAM | similar to | |
| | 96003 | ggtggtggcggaagtgctgatgttcctggccatga | SRILARFSVRRVLLASFLLA | gi\|2072116\| putative | |
| | | gccggatcctcgcgcgcttttcggtccgtcgggtgct | AVRWLLLGAVADHLAVLLF | transporter of | |
| | | gctggccagtttcctcctggccgccgtgcgctggttg | AQVLHAATFASFHASAI(SEQ | 3-phenylpropionic | |
| | | ctgctgggcgcgttggccgatcacctggcggtgct | ID NO:194) | acid [Escherichia coli] | |
| | | gttgttcgcccaggtgctgcacgcggcgacctttgc | | | |
| | | cagctttcacgcctctgccattcatt (SEQ ID | | | |
| | | NO:193) | | | |
| 98. | 13502044/135 | acgacctggtgttctccaagatgctctgcgagcacc | GVLQDALRAPARRPHVSRE | similar to gi\|3327090\| | |
| | 02044 | tgcccgacgcccgcacgtttcacgagaactgcttc | LLQTPSPRRTFGAFLPDPV | KIAA0638 protein | |
| | | aaactccttcgcccaggcggactttcggtgcatttctt | RVPVRGEPAHPRAGGAHR | [Homo | |
| | | cccgaccctgttcgcgttcccgttcgtggtgaacctg | DRQATARAVAEPAPREVPG | sapiens] | |
| | | ctcatccccgagcaggcggcgcgcaccgtgatcg | LLPLDHRPDEEGHQALRKR | | |
| | | gcaagctacagcccgggcggttgcagaacccgc | GFPGRILVRRLRPRILPRTA | | |
| | | accacgagaagttcccggcctactaccgctggac | AARRTRAR(SEQ ID | | |
| | | caccggcccgacgaggaaggccatcaggcgtta | NO: 196) | | |
| | | cgaaagcgtgggtttccaggtcgaatcctggtccg | | | |
| | | gcgcctacggccacggatactaccgcgtactgcc | | | |
| | | gccgctcgacgcactcgagcgcgccaagagccg | | | |
| | | t (SEQ ID NO:195) | | | |
| 99. | 21425684/214 | accggtgatgccaaaggtgctgtgacaaggggat | TGDAKGAVTRGFIGSGKVV | similar to gi\|1881350\| | |
| | 25684 | tcatcggttcgggcaaggtcgtcacggcagctgcc | TAAAVIMISVFVFFIPEGMNA | PROBABLE | |
| | | gtcatcatgatttcggtgttcgtcttcttcatccccgag | IKEIALALAVGILTDAFLVRM | TRANSPORT | |
| | | ggcatgaacgccatcaaggaaatcgccctggccc | TLVPAVMALLGDKAWWLP | PROTEIN, | |
| | | tggccgtcgggatcctcacggatgccttcttggtgc | GWLDRRLPRLDIEGEGITHE | SIMILAR TO | |
| | | ggatgaccctcgtcccggccgtgatggccctgcta | (SEQ ID NO:198) | ANTIBIOTIC | |
| | | ggtgacaaggcatggtggttgcccgggtggctgg | | TRANSPORT- | |
| | | atcgacgcctaccccgcctcgacatcgagggaga | | ASSOCIATED | |
| | | agggatcacccacgaggaaaagctggccgcctg | | PROTEIN | |
| | | gcccacagcggatcacaccgaggccctgcacgc | | ACTII IN | |
| | | cgaggggatcggggtggaggggctcttcgaagg | | STREPTOMYCES | |
| | | cctcgatctgcacgtcgaaccgcgtcaggtgcaag | | COELICOLOR. | |
| | | ccgtcgtcggatcgcagaacagtgtctcggccgtc | | | |
| | | ctgctggcgatcgggggacggctgcccttggatca | | | |
| | | cggccggatgaggtcgggaggattgctgctaccc | | | |
| | | gagcgggcttccagagtgcgtcgggtgacgtggtt | | | |
| | | cctcgacgcgt (SEQ ID NO:197) | | | |
| 100. | 8756491/8756 | tctagatcactctgtagcgcatggttaaatgctgaca | YRATRNAQRNRVLARYEVL | similar to gi\|1351369\| | |
| | 491 | caatagaaaagtgcgaggacatcctcgaattaat | GYLSSGTYGRVYKAK(SEQ | MEIOTIC MRNA | |
| | | gagatggtggactggatgagtcaagttctcgtcgtt | ID NO:200) | STABILITY | |
| | | gcggcggctgtcggtcagctgcccctcctccacttc | | PROTEIN | |
| | | tgcttctcggcgttaccccataccgtattggccgcgt | | KINASE UME5 | |
| | | gttcacctttgaatgcagccatgtcgtcgtctccgtat | | | |
| | | cgaaatgatgtgccatcgaagatgccgacctcag | | | |
| | | catcggcatctgcagtgatgagtgcgtatcgcgcc | | | |
| | | acacgaaacgcccagcgcaaccgtgtcctcgca | | | |
| | | cgatacgaagtgcttgggtatctcagctctggtacct | | | |
| | | atggtcgtgtatataaagcaaaggaactt (SEQ | | | |
| | | ID NO:199) | | | |
| 101. | 2930338/2930 | cggcccccgtggcgaaggagacagccgacgtg | SVKQAAGDWHWALRVSPV | similar to gi\|3874275\| | |
| | 338 | gccagggaggagaagacgtagctgcggtttcga | LGMITGTLILILVPATKRGHV | predicted using | |
| | | atcagggccttcatatctcggagccatgaggtccg | DQLGDQLKARTSWLRDMK | Genefinder; | |
| | | ggccttgagctggtccccgagctggtcgacatgac | ALIRNRSYVFSSLATSAVSF | Similarity to | |
| | | cccttttagtggctgggaccagaatgaggatgagt | ATG(SEQ ID NO:202) | Yeast low-afinity | |
| | | gttcctgtgatcatgcccaggacaggggacaccc | | glucose | |
| | | gcaatgcccagtgccagtctccggctgcctgcttca | | transporter HXT4 | |
| | | cgctg (SEQ ID NO:201) | | (PS:32467); cDNA | |
| | | | EST EMBL:C12555 | | |
| 102. | 20708193/207 | gatcctcacgtcagggcgcttctctcgcaccggag | TLDYFTIDPRLGDDDDFDHL | similar to gi\|3378523\| | |
| | 08193 | gcagcaccgcagcccagaactcaggattgacgg | LQAAHVRGLSVLLDGVVNH | cyclomaltodextrinase | |
| | | aataggcggcgtccagccgccagccgtcaacac | VSRRNRIVQDAQSAGPDSR | | |
| | | cgcgaccgcaccaatagttcatgatccgggtgac | AGRMVRWCEGHLDVFEGH | glucanotransferase | |
| | | atgttcccgcactgcggggttgtcgtggttgagtgcg | SLVALNHDNPAVREHVTRI | [Thermotoga | |
| | | accaggtcactatgaccctcgaaaacgtcgaggt | MNYWCGRGVDGWRLDAA | neapolitana] | |
| | | gcccctcacaccagcgaaccatacggccggcgt | YSVNP(SEQ ID NO:204) | | |
| | | ctgaatctggcccagcactctgcgcatcctgcacg | | | |
| | | atgcggttgcgacgcgagacgtggttgaccaccc | | | |
| | | cgtcgagcagtactgacagaccacgaacgtggg | | | |
| | | cggcctgaagcaggtgatcgaagtcatcgtcgtcg | | | |
| | | cctagccgagggtcgatagtgaagtagtccaaggt | | | |
| | | (SEQ ID NO:203) | | | |
| 103. | 20610403/206 | cgcgtagcggtcgaggttgcggacaccatgcccg | PGLLAIEAPMGHGKTEAAL | similar to gi\|2506493\| | |
| | 10403 | aacccggcctgctcgccatcgaggcacccatggg | MCAQVLAERFGLGGIFFGL | HYPOTHETICAL | |
| | | acacggcaagaccgaggccgccctcatgtgcgc | PTMATSNPMFGRV(SEQ ID | 100.5 KD | |
| | | acaggtgctcgccgaacggttcgggctcggcggc | NO:206) | PROTEIN IN IAP- | |
| | | atcttcttcggtctaccgacgatggccacgtccaatc | | CYSH INTERGENIC | |
| | | ccatgttcggtcgagttcgggaatggctggacgctg | | REGION | |
| | | tgccagccaaggacccgtcaagcatttccctggct | | | |
| | | cactcgaaagctggactcaacgaggagtaccag | | | |
| | | cagctcatgccgtggaacgccaccatggccgtcta | | | |
| | | cgacgaaggtgccggcacgcagcgtgaagct | | | |
| | | (SEQ ID NO:205) | | | |
| 104. | 10268661/102 | actaggggattatcgacataaacgcgactgcgtaa | IIMMAVIAGAVVTNIYCTQPV | similar to gi\|1789035 | |
| | 68661 | ggttggtgactcatcccccagcgacatgaggcaa | LPLIASDMGVAVSTVNLVAG | orf, hypothetical | |
| | | acgccatgacatccgagaatgcaccgccgcgag | AALLGFATGLAFLLPMGD | protein | |
| | | gcaagatcatcatgatggcggtgatcgccggcgc | (SEQ ID NO:208) | [Escherichia coli] | |
| | | ggtggtcaccaacatttactgcacccagccggtgct | | | |
| | | gccgttgatcgcctcggacatgggcgtcgcagtgt | | | |
| | | cgacggtcaacctggtggcaggcgcggccttgct | | | |
| | | ggggtttgccaccgggttggcgtttttattgcccatgg | | | |
| | | gcgaccgctt (SEQ ID NO:207) | | | |
| 105. | 19536322/195 | agtacttgtcatgattacgcctagtttgggtatctatttc | YFSQRSQISRTQDDEARTR | similar to gi\|1870004\| | |
| | 36322 | tctcagcgttctcagatctcccgaacccaagacga | ASISTLQDEVKRWHDPDYV | hypothetical protein | |
| | | cgaggctcggacacgcgcttctatctcgacccttca | RAQARSQLGWVMPGETGY | Rv1024 | |
| | | agacgaggtcaagaggtggcacgatcccgacta | QV(SEQ ID NO:210) | [Mycobacterium | |
| | | cgtccgtgctcaggcgcgctcccagctcggctggg | | tuberculosis] | |
| | | tgatgccgggcgaaactgggtatcaggtcattgga | | | |
| | | gaaaacggtaaggtcattggatcgacgacttctttg | | | |
| | | gacgaaaaagatccggcgagtgaagccagcgct | | | |
| | | gacgctcggtggtggcaagaggcttgcggatcagt | | | |
| | | c (SEQ ID NO:209) | | | |
| 106. | 3150182/315 | cccggctgcatgaccggtgtggcgaccaacgcgg | GCMTGVATNAVFGSGLRA | similar to gi\|131498\| | |
| | 182 | gttcggttccggccttcgcgctcccgcatcgcccg | PASPGSIFAVLLQT(SEQ ID | PTS SYSTEM, | |
| | | gatcgatcttcgcggtgctgttgcagacctccggcg | NO:212) MANNITOL- | | |
| | | acagcaactt (SEQ ID NO:211) | | SPECIFIC IIABC | |
| | | | | COMPONENT | |
| | | | | (EIIABC-MTL) | |
| | | | | (MANNITOL- | |
| | | | | PERMEASE IIABC | |
| | | | | COMPONENT) | |
| | | | | (PHOSPHOTRANSF | |
| | | | | ERASE ENZYME II, | |
| | | | | ABC | |
| 107. | 13515411/135 | gtgcacatccggacgaaaattgctgctttcaatcaa | LLSIKSDVYSYGVLVLEIITG | similar to gi\|836954 | |
| | 15411 | gtccgacgtgtacagctacggggtgttggtgctgga | HK(SEQ ID NO:214) | receptor protein | |
| | | gatcatcaccggccacaagatct (SEQ ID | | kinase | |
| | | NO:213) | | [Ipomoea trifida] | |
| 108. | 4526774/4526 | gattttggtttatcaaggcttattggtgaagggaatac | DFGLSRLIGEGNTRIVNTHT | similar to gi\|2911080\| | |
| | 774 | tagaatagtcaacacacacactcctggaccaata | PGPIGYMAPEYRYMGEVSR | receptor kinase-like | |
| | | gggtacatggcacctgaataccgttacatggggga | KVDIFSLGILILEIVTGQDNS | protein | |
| | | agtttcacgcaaggtagacatattcagcttgggtatt | (SEQ ID NO:216) | [Arabidopsis thaliana] | |
| | | cttatactagagaffgtaactggtcaagacaatagt | | | |
| | | accaaagttgactttatcgatcatgtatgtgaacact | | | |
| | | gcatggaagggccacaaataaaatttatgtat | | | |
| | | (SEQ ID NO:215) | | | |
| 109. | 3793822/3793 | acgcgtactgagtatcggtatcgatgttcatcttgata | YTTVADGMRTLEVLGLGEK | similar to gi\|4049524\| | |
| | 822 | acgccataggacaccgcgtcggcgatctcctggg | GRYTTALTFGNVHGAYKPG | fructose 1,6- | |
| | | cagtcgatcccgacccaccatggaagaccagatc | YVKLRPGILKEIQDECGKKY | bisphosphate | |
| | | gaacggcttctccttgccgtacttcttgccgcactcgt | GKEKPFDLVFHGGSGSTAQ | aldolase | |
| | | cctggatctccttgaggattcccgggcgcagcttga | EIADAVSYGVIKMNIDTDTQ | [Streptomyces | |
| | | cgtaacccggcttgtacgcaccatggacgttcccg | YA(SEQ ID NO:218) | galbus] | |
| | | aaggtcagagcggtggtgtagcgacccttctcacc | | | |
| | | caggcccagcacctcgagggtacgcatgccgtca | | | |
| | | gcaacggtggtgtaca (SEQ ID NO:217) | | | |
| 110. | 3670835/3670 | agcttgcgcccgatgacctaacagatcatggctag | GVALGGPFTLTDQDGRVRT | similar to | |
| | 835 | tcgcgctatgaaccagatcgcccgtgcgctttccgc | DRDFAGRYRIMYFGYTFCP | gi\|2126490\| | |
| | | gctgtttcttgctgttttaccggtcggctgtagcgggc | DVCPTDM(SEQ ID NO:220) regulatory protein | | |
| | | ccgccacaccggcgcgcgcaccgctggacggc | | prrC - | |
| | | gttgcgcttggcggtccgttcacactgaccgaccag | | Rhodobacter | |
| | | gatggccgggtgcgcaccgatcgcgatttcgccg | | sphaeroides | |
| | | ggcgctatcgcatcatgtatttcggctacaccttttgc | | | |
| | | cccgatgtgtgcccgaccgacatgcagacgattg | | | |
| | | gcgcgggcttacgcg (SEQ ID NO:219) | | | |
| 111. | 2873001/2873 | ggatccacatgcccagggcccccgtggcgaagg | LPAVFICSFMVAAPIFGYLG | similar to | |
| | 001 | agacagccgacgtggccagggaggagaagacg | DRFNRKVILSCGIFFWSAVT | gi\|3874275\| predicted | |
| | | tagctgcggtttcgaatcagggccttcatatctcgga | FSSSFIPQQYFWLLVLSRGL | using Genefinder; | |
| | | gccatgagggccgggccttgagctggtccccgag | VGIGEASYSTIAPTIIGDLFT | Similarity to | |
| | | ctggtcggcatgaccccttttagtggctgggaccaa | KNTRTLMLSVFYFAIPLGSG | Yeast low-afinity | |
| | | aatgaggatgagtgttcctgtgatcatgcccaggac | LGYITGSSVKQAAGDWHW | glucose | |
| | | aggggacacccgcaatgcccagtgccagtctccg | ALRVSPVLGMITGTLILILVP | transporter HXT4 | |
| | | gctgcctgcttca (SEQ ID NO:221) | ATKR(SEQ ID NO:222) | (PS:32467); cDNA | |
| | | | | EST EMBL:C12555 | |
| 112. | 3226165/3226 | ctgcaaccggtgggtatcgtccataccaccggcca | PRAVMPIHGEVRHLVANAD | similar to gi~341 38281 | |
| | 165 | tcgccttctctagctcggtgacgatctggtcggtgat | LAKATGVDENNWLVEDGG | hypothetical protein | |
| | | ctccgcgaagaccgagtcgccctcggcaaaacc | VIDLVDGVPRVVGKVDASYI | SC9A1 0.09 | |
| | | acgcgcctggatcgccgggcgagacaccactga | LVDGSGVGELTEDTLTDRRI | [Streptomyces | |
| | | cgccgagcgggtgtcgaccacggtgacgactga | LGEEGFLSVVTVVDTRSAS | coelicolor] | |
| | | caagaatccctcctcaccgaggatacggcgatca | VVSRPAIQARGFAEGDSVF | | |
| | | gtgagcgtgtcctcggtaagctcccccaccccaga | AEITDQIVTELEKAMAGGMD | | |
| | | tccgtcaacaaggatgtacgaggcatcgaccttgc | DTHRLQ(SEQ ID NO:224) | | |
| | | caacaactcgcggtactccgtcaacaaggtcaat | | | |
| | | aaccccgccgtcctcgacaagcaccacgttgttct | | | |
| | | catcgacaccggttgctttggccagatcggcattag | | | |
| | | cgacaagatgacgcacctcaccatgaatcggcat | | | |
| | | cacagcgcgtggc (SEQ ID NO:223) | | | |
| 113. | 3928075/3928 | ccgcactgtggcgaccatccttgccaccattaccat | ANGEDEVSRKLITVWGAEP | similar to | |
| | 075 | tgccgccctagtgctcacgggctgtaatacggcgg | QNPLLPADTNETGGTKVITA | gi\|2960090\|dppA | |
| | | tgcgcnaaacggtgaagacgaggtttcccgcaag | LFAGLVYYDADGKTHNDVA | [Mycobacterium | |
| | | ctcatcaccgtgtggggtgctgagccacaaaaccc | KSIDFDGDRTYTVTLR(SEQ | tuberculosis] | |
| | | actcctgccagccgacaccaatgaaaccggcgg | ID NO:226) | | |
| | | cacgaaagtcatcaccgccttgttcgccggcctggt | | | |
| | | gtattacgacgccgacggcaaaaacccataatga | | | |
| | | tgtggccaaatccattgacttcgatggcgaccgca | | | |
| | | cctacacggtgacgctgcggaaaaccagat | | | |
| | | (SEQ ID NO:225) | | | |
| 114. | 20286807/202 | cgccgattaccaaggctatggatgtgtgggccttgg | ITKAMDVWALGVTLYCLLFG | similar to | |
| | 86807 | gcgtaacgctatactgtctgctgttcggtcgagtgcc | RVPFDAETEYLLLESILHDD | gi\|1711543\| | |
| | | atttgatgcagagacggagtacttgctgctggaaa | YAVPT(SEQ ID NO:228) | SERINE/THREONIN | |
| | | gtatcctgcatgacgattatgccgtcccgacgcaca | | E-PROTEIN | |
| | | tgggtagcgaccgcgtgttggtaggcccgcgacc | | KINASE SSPI | |
| | | agcacgttggccctcgtcgcaagagacgcccaac | | | |
| | | gtgccgctgtccggcgaggcgcatgcagtacgcc | | | |
| | | atctgctcgatgcccttctcgacaaggatccagcga | | | |
| | | cgcgcctcactctcgatcgtgttataacacacccat | | | |
| | | ggctcgtggcagaatcatggtaatagtagcagttgt | | | |
| | | atataccctcatcaccaagatggccaaagcggta | | | |
| | | caaggtacgcg (SEQ ID NO:227) | | | |
| 115. | 20944739/209 | tcggtcttgaacccttccagatcttcttctttatcattctg | FIILPIMAGGVGEGAIPLSIG | similar to gi\|1783249\| | |
| | 44739 | ccgattatggccggtggcgttggtgagggcgctatt | YATLLHMDQGVaLGRVLPM | homologous to | |
| | | ccgctctctatcggatatgccacgttgctgcatatgg | VMLGGLTAIIISGCLNQLGK | citrate-sodium | |
| | | atcagggcgttgcgctcggtcgcgtgctgccgatg | RYPHLT(SEQ ID NO:230) | symport (citrate | |
| | | gtcatgctcggaggcttaaccgccatcattatctccg | | transporters); | |
| | | gctgcctgaaccagcttggtaaacgctatccgcatc | | hypothetical | |
| | | tgaccg (SEQ ID NO:229) | | [Bacillus subtilisi | |
| 116. | 2974211/2974 | ccgcgtgaagggcagcagcaacaccacggagt | GSSNTTECVPVPTSEHVAEI | similar to gi\|2088718 | |
| | 211 | gtgttcccgtgcccacctccgagcacgtggccgag | VGRQGCKIKALRAKTNTYIQ | weak similarity to | |
| | | atcgtgggcaggcaaggctgcaagattaaggcctt | (SEQ ID NO:232) | human | |
| | | gagggccaagaccaacacctacattcaa (SEQ | | transformation | |
| | | ID NO:231) | | upregulated nuclear | |
| | | | protein | | |
| | | | (NID:g460789) | | |
| | | | [Caenorhabitis | | |
| | | | elegans] | | |
| 117. | 10286661/102 | acgcgtgcaacgcctcgatgaccgccgctgccat | RDDQVQHCTDNREEMQRA | similar to gi 13128335 | |
| | 86661 | cgcaaacacgccggggtcgccggacgacacca | RHAFELAAQGRSVVVVSSG | precorrin-3 | |
| | | ccaccactgagcgcccttgcgccgccagctcaaa | DPGVFAMAAAVIEALHA | methylase | |
| | | cgcatgccgcgcgcgctgcatttcttcgcggttatcg | (SEQ ID NO:234) | [Rhodobacter | |
| | | gtgcaatgctgcacctgatcatcacggaacggccc | | capsulatus] | |
| | | ggccatgcgcacgtaggtttcatagccca (SEQ | | | |
| | | ID NO:233) | | | |
| 118. | 20945165/209 | gcatggcctcggaaccgagttcgtcctcgcgttcct | RNAEQRKNVLKYDDVLNRQ | similar to gi\|2251198 | |
| | 45165 | ggtagacgagcttggcgtccgagaggatctgttcc | REVVYRDRRRILMGDDIAE | SecA [Streptomyces | |
| | | gtgaggaagtccgcggtcaaccgcgggcgtccg | QIQQFVEEVITTAIEDRTRV | lividans] | |
| | | ccggcctcgtcaatgatttcctgctgggtcaacgtg | GHSEDWDLDGLWEALREL | | |
| | | atggggtacagctcacgcaatgcctcccacagac | YPITLTQQEIIDEAGGRPRLT | | |
| | | cgtccagatcccagtcctcggaatgacccacgcg | ADFLTEQILSDAKLVYQERE | | |
| | | agtgcggtcttcgatggcggtggtgatgacctcttcc | DELGSEAM(SEQ ID | | |
| | | acgaactgctgaatctgttcggcaatgtcatcgccc | NO:236) | | |
| | | atgaggattcgacggcggtcacggtagaccacttc | | | |
| | | gcgctggcggttgagcacgtcgtcgtacttgagca | | | |
| | | cattcttgcgctgctcggcgttacg (SEQ ID | | | |
| | | NO:235) | | | |
| 119. | 3927847/3927 | tccgaccgcgtggtaaaactggcgaccttaattgct | RVVKLATLIAEDEQAEMNIV | similar to gi\|2506682\| | |
| | 847 | gaagatgagcaagctgaaatgaatattgttttgccc | LPAAWLHDCVSYPKNHVLR | HYPOTHETICAL | |
| | | gcagcgtggttgcatgattgcgtcagttaccctaaa | AQSALHAADKAIVFLRSINY | 25.9 KD | |
| | | aaccatgtattaagagcacaaagtgcattacatgc | PKQYLLAIHHAISAHSVSGKI | PROTEIN IN | |
| | | agcagataaagcgattgtatttttgcgcagtattaatt | QAMSLEAQIVQDADRLDAL | DCM-SERU | |
| | | accccaaacaatacttattagcaattcatcatgcaa | GAIGVARCIQVSSQLQRPLY INTERGENIC | | |
| | | tttcagcgcacagtgtcagtggtaaaatacaggca | SEVDPFSETRSL(SEQ ID | REGION | |
| | | atgagtttagaagctcaaatagtgcaagatgcaga | NO:238) | | |
| | | tagattggatgcgctaggggcaattggcgtggctc | | | |
| | | gttgcattcaagtaagtagccagttacagcgccca | | | |
| | | ctatattctgaagttgaccccttcagcgagacacga | | | |
| | | tctctag (SEQ ID NO:237) | | | |
| 120. | 4033506/4033 | tctagattgaactcgaccgcacaggtgcgctgagg | PWHPGRCAALIVDGKVIGH | similar to | |
| | 506 | cagaccagccttcgacactactgtggggtgcaact | AGELHPTVVSKAGLPQRTC | gi\|3122893\| | |
| | | caccagcatggccaatgaccttcccatcgacgatg | AVEFNL(SEQ ID NO:240) | PHENYLALANYL- | |
| | | agagccgcacaacgaccgggatgccatggagc | | TRNA | |
| | | ctgacggtcagccttgcgcacc (SEQ ID | | SYNTH ETASE | |
| | | NO:239) | | BETA CHAIN | |
| | | | (PH ENYLALAN INE-- | | |
| | | | TRNA LIGASE | | |
| | | | BETA CHAIN) | | |
| | | | (PHERS) | | |
| 121. | 21643932/216 | gccggcgtgttcaacctcatggtgtgggccttcatta | NLMVWAFITDVIDAQEVMS | similar to gi\|3915488\| | |
| | 43932 | ccgacgtcatcgatgcccaggaggtcatgtccgg | GEREDGVIYGVNSFARKLA | HYPOTHETICAL | |
| | | ggagcgtgaagacggtgtcatctatggcgtgaact | QAIAGGIGGAMLTMIGY | SYMPORTER IN | |
| | | ccttcgcccgcaaacttgcccaggccattgccggt | (SEQ ID NO:242) | COTT-RAPA | |
| | | ggaatcggcggagccatgctgacgatgatcggct | | INTERGENIC | |
| | | accagtcctcctcccaaggtggtgccgttcagtcgg | | REGION | |
| | | agtccgtcgtcaatcacctgtacacgctcgccacc | | | |
| | | gccatcccgacgatctgctgcctcggcgctgccct | | | |
| | | gctcatgctgggctacccgctcacccgcgacaag | | | |
| | | gtggtcgccaacgccgacgagttggctcgtcgcca | | | |
| | | cgcagtacaggccgagcaaaactcctgacccata | | | |
| | | acggaggcacatcatggacacgctcatgcggatc | | | |
| | | accgaccacttgacaacctcgccgggtatccaatt | | | |
| | | gaaaattgacaagcgatggggtgcctccgtcacat | | | |
| | | ttgtgacgcgt (SEQ ID NO:241) | | | |
| 122. | 2797911/2797 | cgatcctgcacaaagggtgcgttttctcgctgtcctc | DPAQRVRFLAVLEHVPDDR | similar to gi\|4587878 | |
| | 9114 | gagcatgttcctgatgatcgcgtcatcgtggtgtcca | VIVVSTHQVDDLTDSYDRVI | ABC transporter | |
| | | ctcaccaggttgacgacctcactgacagctatgac | VLSCGRVVFDGTPKEFVDL( | subunit PenJ | |
| | | agggtgattgtactgtcatgtggccgggtcgtcttcg | SEQ ID NO:244) | [Pediococcus | |
| | | acggaacccctaaggagttcgtggatctggcgcc | | pentosaceus] | |
| | | agagtgttcgcagcgtcgtgctgaaatggcctacct | | | |
| | | cggtctggtggccgacgaatgaggctcacaacgc | | | |
| | | ggctgcgaactgcgcccggtgtctggttggcgccc | | | |
| | | ctgatggtcgtggccttcatcatggttgtcaatgaca | | | |
| | | ccgccggtgagccgtattggttagctcagtggccg | | | |
| | | gggacgcggggagaaccatggtcgtcaacgcgc | | | |
| | | (SEQ ID NO:243) | | | |
| 123. | 20941626/209 | caatggctggcggtgatctgcgggtttgtcggtgtgc | QWLAVICGFVGVLIIIHPGGE | similar to gi\|3860646 | |
| | 41626 | tgatcatcatccaccccggtggcgagttgttcacgc | LFTPAVLLPLCSAMFFCFYQ | unknown [Rickettsia | |
| | | ctgcggtgttgctgccgctgtgttcggcgatgttcttct | LLTRILSQYDTPTTSNFFAG | prowazekii], also | |
| | | gtttctaccaattgctcacgcgcatcctcagccaata | LCNTLVMSALVPFFWQVPT | to OCTAPEPTIDE | |
| | | cgacacaccgaccaccagcaacttcttcgccggg | LWHACLMLALGTCG(SEQ | REPEAT PROTEIN | |
| | | ctgtgcaataccttggtgatgagcgcgctggtgccg | ID NO:246) | T2 | |
| | | ttcttctggcaggtcccgacgttgtggcatgcctgttt | | (mouse) | |
| | | gatgctggcgctgggcacttgcggcatgaccgcg | | | |
| | | cact (SEQ ID NO:245) | | | |
| 124. | 37004299/370 | cgcgcccagaggccgctgagcccaggaagaag | RKKKWTLSLKNLRPEDSGK | similar to gi\|1168177 | |
| | 04299 | aagtggacactgagcctgaagaacctgcggccg | YTCRVSNRAGAINATYKVD | fibroblast growth | |
| | | gaggacagcggcaaatacacctgccgcgtgtcg | VIQRTRSKPVLTGTHPVNTT | factor | |
| | | aaccgcgcgggcgccatcaacgccacctacaag | VDFGGTTSFQCKVRSDVKP | receptor type 1, | |
| | | gtggatgtgatccagcggacccgttccaagcccgt | VIQWLKRVE(SEQ ID | FGFR1 | |
| | | gctcacaggcacgcaccccgtgaacacgacggt | NO:248) | {Val423,Thr424 | |
| | | ggacttcggggggaccacgtccttccagtgcaag | | deletion} | |
| | | gtgcgcagcgacgtgaagccggtgatccagtggc | | [Xenopus | |
| | | tgaagcgcgtggagtacggcgccgagggccgcc | | laevis = African clawed | |
| | | acaactccaccatcgatgtgggcggccagaagttt | | frogs, embryos, | |
| | | gtggtgctgcccacgggtgacgtgtggtcgcggcc | | Peptide Mu | |
| | | cgacggctcctacctcaataagctgctcatcaccc | | | |
| | | gtgcccgccaggacgatgcgggcatgtacatctg | | | |
| | | ccttggcgccaacaccatgggctacagcttccgca | | | |
| | | gcgccttcctcaccgtgct (SEQ ID NO:247) | | | |
| 125. | 326847/32684 | ggtttccgtcagatttctctgaacaaggtggccgtcc | GFRQISLNKVAVLLLAGGQ | similar to gi\|4507759 | |
| | 7 | tgctgctggctggggggcagggcactcgcctggg | GTRLGVTYPKGMYRVGLPS | sperm associated | |
| | | cgtgacctaccccaagggtatgtaccgtgtggggc | RKTLYQLQAERIRRVEQLA | antigen 2 | |
| | | tgcccagccggaagaccctgtaccagctgcaggc | GERHGTRCTV(SEQ ID | | |
| | | ggagcggattcggcgggtggagcagctggccggt | NO:250) | | |
| | | gagcgccacgggacccgctgcaccgtcc (SEQ | | | |
| | | ID NO:249) | | | |
| 126. | 29914845/299 | cgatccattcattataagctgcaaagagacaaatat | LFNFQANRDELTNGIIHAAF | similar to gi\|2792502 | |
| | 14845 | aatctttaaagaggagcataaaagcagcatgtatt | MLLFKDYICLFAAYNEWI(SEQ | clathrin assembly | |
| | | ataccattagttagttcatcacgatttgcctgaaaatt | ID NO:252) | protein | |
| | | aaataaaaacgtaaccaatta (SEQ ID | | long form [Rattus | |
| | | NO:251) | | norvegicus] | |
| 127. | 27365065/273 | tgcactctctgttcctgctcattctcacagcccatgtta | NALEEAGCWADVWALQLQ | similar to gi\|2492916 | |
| | 65065 | gaaagcgaacgtttgaccctctttggacctgcctgc | LYRFGGVEATQALIRHLPEL | APOLIPOPROTEIN | |
| | | tcccaggctaagagctgtagggcagaggacaga | QKGGCADWKVSVNALSSA | F PRECURSOR | |
| | | gcattcacggacactttccagtctgcacatccacctt | LQLLAWEQAGPKRVKRSLS | (APO-F) | |
| | | tctggagctctggaagatggcggatgagggcctgt | NMGCENEQEQRV(SEQ ID | | |
| | | gtagcctccacacctccgaatcggtaaagctgaa | NO:254) | | |
| | | gctgaagagcccagacatcagcccagcagccag | | | |
| | | cttcctccagagcattcc (SEQ ID NO:253) | | | |
| 128. | 30575179/305 | ccggacagaggtttcgcaagcgactgtcctcgctc | GQRFRKRLSSLRRVVGGLV | similar to gi\|4165317 | |
| | 75179 | aggcgcgtggtgggcggactagtggctctgcctgg | ALPGSHPYIAALYWGDSFC | FXII [Sus scrofa] | |
| | | atcgcatccctacatcgctgcactatactggggcga | AGSLIDPCWVLTAAHCLQK | | |
| | | gagcttctgcgcaggcagtctcatcgacccctgctg | RPAPEELTVVLGQDRHNQS | | |
| | | ggtgctgaccgctgctcactgcttgcagaaacggc | CERCQTLAV(SEQ ID | | |
| | | cagcgcccgaagaactgacagtggtacttggtca | NO:256) | | |
| | | agatcgccataaccagagctgcgagaggtgcca | | | |
| | | gactctggctgtgca (SEQ ID NO:255) | | | |
| 129. | 17676856/176 | agccttggtagtggtcccggggaatgggagccac | EDCVMMLHSGLWNNAPCA | similar to gi\|560484 | |
| | 76856 | actctgcttcacgccgaggcaatgggcgtggtcag | NERDGWICEKRSSC(SEQ Fc-E receptor II | | |
| | | caactgctcctcttctcgcaaatccagccgtctctctc | ID NO:258) | (Fc-ERI l/CD23) | |
| | | attggcacatggcgcattattccacagccccgagt | | [Mus musculus] | |
| | | gcaacatcatgacacaatcctcatgccctctgg | | | |
| | | (SEQ ID NO:257) | | | |
| 130. | 7511820/7511 | caccgactcctcgtctcccggggccaagggacgg | SNEVEAVNGTEVRLKCTFK | similar to gi\|5032247 | |
| | 820 | aagttccaggagacagtcacagaggagaggga | SKHPVSPSSVTVSWNFRPL epithelial V-like | | |
| | | cacagggtgtttggacttaaaagtgcacttcagtctc | APGDEESV(SEQ ID | antigen | |
| | | acttctgtcccgttcactgcctccacctcattgga | NO:260) | | |
| | | (SEQ ID NO:259) | | | |
| 131. | 38406286/384 | gtgcactggggtcagggtttctcacacttcccacaa | ISDQSLVVSSVQHQSTMEL | similar to gi\|2500773 | |
| | 06286 | agaggggtatacctatggtctcctccatgatgaaga | SEAGVEAAAATSTAMTRMS | ALPHA-2- | |
| | | agatgaatggtcggttcaaaaagaaggaggaaa | LSSFFLNRPFIFFIMEETIGIP | ANTIPLASMIN | |
| | | gggacattcgagtcatggctgtgctggtagctgccg | LFVGSVRNPDPSA(SEQ ID | PRECURSOR | |
| | | ctgcctccacaccagcctcactgagctccatggta | NO:262) | (ALPHA-2-PLASMIN | |
| | | gactggtgctgcacactagacaccaccagactctg | | INHIBITOR) (ALPHA- | |
| | | gtcagagatct (SEQ ID NO:261) | | 2-PI) | |
| | | | (ALPHA-2-AP) | | |
| 132. | 25236336/252 | atatcaagaatgttataattcaagaaaactaccatt | IKNVIIQENYHYPAHDNDIAV | similar to gi\|4758508 | |
| | 36336 | accctgcacacgataatgacattgctgttgtgcatct | VHLSSPVLYTSNIQKACLPN | airway trypsin-like | |
| | | atcttcaccagtgttatatacaagcaacatccaaaa | VNYIFLYNSEAVVTAW(SEQ | protease | |
| | | agcatgtcttccaaatgttaattatatattcctatacaa | ID NO:264) | | |
| | | ttcagaagcagtggttactgcatgggg (SEQ ID | | | |
| | | NO:263) | | | |

Equivalents

From the foregoing detailed description of the specific embodiments of the invention, it should be apparent that particular novel compositions and methods involving nucleic acids, polypeptides, antibodies, detection and treatment have been described. Although these particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made as a matter of routine for a person of ordinary skill in the art to the invention without departing from the spirit and scope of the invention as defined by the claims. Indeed, various modifications of the invention in addition to those described herein will become apparent to those skilled in the art from the foregoing description and accompanying figures. Such modifications are intended to fall within the scope of the appended claims.

What is claimed is:

1. An isolated nucleic acid molecule encoding a polypeptide comprising an amino acid sequence that is at least 85% identical to a polypeptide including an amino acid sequence selected from the group consisting of SEQ ID NO: 2n, wherein n is any integer 1-30, or the complement thereof.

2. The isolated nucleic acid molecule of claim 1, said molecule hybridizing under stringent conditions to a nucleic acid sequence complementary to a nucleic acid molecule comprising the sequence of nucleotides selected from the group consisting of SEQ ID NO: 2n-1.

3. The isolated nucleic acid molecule of claim 1, said molecule encoding a polypeptide comprising the amino acid sequence selected from the group consisting of SEQ ID NO: 2n, or an amino acid sequence comprising one or more conservative substitutions in the amino acid sequence selected from the group consisting of SEQ ID NO: 2n.

4. The isolated nucleic acid molecule of claim 1, wherein said molecule encodes a polypeptide comprising the amino acid sequence selected from the group consisting of SEQ ID NO: 2n.

5. The isolated nucleic acid molecule of claim 1, wherein said molecule comprises the sequence of nucleotides selected from the group consisting of SEQ ID NO: 2n-1 or the complement thereof.

6. An oligonucleotide less than 100 nucleotides in length and comprising at least 6 contiguous nucleotides selected from the group consisting of SEQ ID NO: 2n-1 or the complement thereof.

7. A vector comprising the nucleic acid molecule of claim 1.

8. The vector of claim 7, wherein said vector is an expression vector.

9. A host cell comprising the isolated nucleic acid molecule of claim 1.

10. A substantially purified polypeptide comprising an amino acid sequence at least 80% identical to a polypeptide comprising the amino acid sequence selected from the group consisting of SEQ ID NO: 2n, wherein n is any integer 1-30.

11. The polypeptide of claim 10, wherein said polypeptide comprises the amino acid sequence selected from the group consisting of SEQ ID NO: 2n.

12. An antibody that selectively binds to the polypeptide of claim 10.

13. A pharmaceutical composition comprising a therapeutically or prophylactically effective amount of a therapeutic selected from the group consisting of the nucleic acid of claim 1.

14. A pharmaceutical composition comprising a therapeutically or prophylactically effective amount of a therapeutic selected from the group consisting of the antibody of claim 12.

15. A pharmaceutical composition comprising a therapeutically or prophylactially effective amount of a therapeutic selected from the group consisting of the antibody of claim 12.

16. A kit comprising in one or more containers, a therapeutically or prophylactically effective amount of the pharmaceutical composition of claim 13.

17. A method of detecting the presence of the polypeptide of claim 10 in a sample, comprising contacting the sample with a compound that selectively binds to said polypeptide under conditions allowing the formation of a complex between said polypeptide and said compound, and detecting said complex, if present, thereby identifying said polypeptide in said sample.

18. A method of detecting the presence of a nucleic acid molecule of claim 1 in a sample, the method comprising contacting the sample with a nucleic acid probe or primer that selectively binds to the nucleic acid molecule and determining whether the nucleic acid probe or primer bound to the nucleic acid molecule of claim 1 is present in the sample.

19. A method for modulating the activity of the polypeptide of claim 10, the method comprising contacting a cell sample comprising the polypeptide of claim 10 with a compound that binds to said polypeptide in an amount sufficient to modulate the activity of the polypeptide.

20. A method for screening for a modulator of activity or of latency or predisposition to an ORFX-associated disorder, said method comprising:
a) contacting a test compound with the polypeptide of claim 10; and
b) determining if said test compound binds to said polypeptide, wherein binding of said test compound to said polypeptide indicates the test compound is a modulator of activity or of latency or predisposition to an ORFX-associated disorder.

21. A method for screening for a modulator of activity or of latency or predisposition to an ORFX-associated disorder, said method comprising:
a) administering a test compound to a test subject at an increased risk ORFX-associated disorder, wherein said test subject recombinantly expresses a polypeptide encoded by the nucleotide of claim 1;
b) measuring expression the activity of said protein in said test subject;
c) measuring the activity of said protein in a control subject that recombinantly expresses said protein and is not at increased risk for an ORFX-associated disorder; and
d) comparing expression of said protein in said test subject and said control subject, wherein a change in the activity of said protein in said test subject relative to said control subject indicates the test compound is a modulator or of latency of predispostition to an ORFX-associated disorder.

22. The method of claim 20, wherein said test animal is a recombinant test animal that expresses a test protein transgene or expresses said transgene under the control of a promoter at an increased level relative to a wild-type test animal, and wherein said promoter is not the native gene promoter of said transgene.

23. A method for determining the presence of or predisposition to a disease associated with altered levels of a polypeptide of claim 11 in a subject, the method comprising:
a) measuring the amount of the polypeptide in a sample from said subject; and
b) comparing the amount of said polypeptide in step (a) to the amount of the polypeptide present in a control sample,
wherein an alteration in the level of the polypeptide in step (a) as compared to the control sample indicates the presence of or predisposition to a disease in said subject.

24. The method of claim 23, wherein said subject is a human.

25. A method for determining the presence of or predisposition to a disease associated with altered levels the nucleic acid molecule of claim 1 in a subject, the method comprising:
a) measuring the amount of the nucleic acid in a sample from the mammalian subject; and
b) comparing the amount of said nucleic acid in step (a) to the amount of the nucleic acid present in a control sample,
wherein an alteration in the level of the nucleic acid in step (a) as compared to the control sample indicates the presence of or predisposition to said disease in said subject.

26. The method of claim 25, wherein said subject is a human.

27. A method of treating or preventing a pathological condition associated with an ORFX-associated disorder in a subject, the method comprising administering to said subject the polypeptide of claim 10 in an amount sufficient to alleviate or prevent said pathological condition.

28. The method of claim 27, wherein said subject is a human.

29. A method of treating or preventing a pathological condition associated with an ORFX-associated disorder in a subject, the method comprising administering to said subject the nucleic acid molecule of claim 1 in an amount sufficient to alleviate or prevent said pathological condition.

30. The method of claim 29, wherein said subject is a human.

31. A method of treating or preventing a pathological condition associated with an ORFX-associated disorder in a subject, the method comprising administering to said subject the antibody of claim 12 in an amount sufficient to alleviate or prevent said pathological condition.

32. The method of claim 31, wherein said subject is a human.
